(19) United States
(12) Patent Application Publication
(10) Pub. No.: US 2025/0114324 A1
(43) Pub. Date: Apr. 10, 2025

(54) ANTIMICROBIAL MATERIALS AND NANOPARTICLES AND METHODS OF USE THEREOF

(71) Applicant: Arizona Board of Regents on behalf of Arizona State University, Scottsdale, AZ (US)

(72) Inventors: Shu Wang, Phoenix, AZ (US); Zhaoyang Fan, Phoenix, AZ (US)

(21) Appl. No.: 18/833,613

(22) PCT Filed: Feb. 10, 2023
(86) PCT No.: PCT/US23/62346

§ 371 Date: Jul. 26, 2024

Related U.S. Application Data
(60) Provisional application No. 63/309,310, filed on Feb. 11, 2022.

(51) Int. Cl. A61K 31/355; A23B 7/16; A23L 33/15; A61K 9/51; A61K 45/06

(52) U.S. Cl. CPC A61K 31/355; A23B 7/16; A23L 33/15; A61K 9/5123; A61K 45/06

(57) ABSTRACT

The present invention provides antimicrobial nanoparticles or compositions thereof comprising at least one phytochemical or bioactive compound, at least one lipid, at least one surfactant, and at least one vitamin E. In some embodiments, the antimicrobial nanoparticles or compositions thereof further comprise at least one coating agent. In some embodiments, the antimicrobial nanoparticles or compositions thereof are an edible coating. In various aspects, the present invention relates to methods of reducing or inhibiting the activity or level of or preventing the growth of at least one microorganism on a surface of an element. In some aspects, the present invention relates to methods of increasing a shelf-life of food and/or maintaining and improving food nutrients and/or nutrition values. In other aspects, the present invention relates to methods of delivering at least one nutritional or bioactive agent for preventing and treating various disorders and diseases.

### CROSS REFERENCE TO RELATED APPLICATIONS

This application claims priority to U.S. Provisional Application No. 63/309,310, filed Feb. 11, 2022, which is hereby incorporated by reference herein in its entirety.

### BACKGROUND OF THE INVENTION

Many natural phytochemicals from plants express antioxidant, antimicrobial, antigenotoxic, anti-inflammatory, and antidiabetic functions, are attractive healthy supplements. Many of them, however, have a low level of solubility, stability, bioavailability, and a high level of hepatic metabolism. Most of these phytochemicals not only have health benefit, but also have antimicrobial and antioxidant activities to actively protect the food if applied on foods as food packaging materials.

Many foods like fresh fruits and vegetables, meats, dairy, chocolate, and others are popular foods. However, they are highly perishable. A short shelf life, waste generation, and hence high cost, as well as foodborne illness are associated problems that must be solved. Edible coating based active packaging, which aims to provide both a physical barrier against dehydration and gas permeation and chemical protection against microbes and other deleterious processes, has gained traction, particularly if using natural phytochemicals as antioxidants and antimicrobials. Nevertheless, the free form of some compounds in the coating is also easily degraded. To compensate for the performance degradation over time, large quantities need to be applied, which is not only very costly but can also negatively affect the sensory properties of foods. Consumer's perception and the high cost are barriers to the largescale acceptance of active packaging.

Thus, there is a need in the art for antimicrobial coating that can improve the stability of phytochemicals, provide a carrier for nutraceutical supplements, and maintain the quality and safety of foods, extending their shelf life of perishable foods. The present invention satisfies this unmet need.

### SUMMARY OF THE INVENTION

In one aspect, the present invention provides a nanoparticle comprising a) at least one phytochemical or bioactive compound, wherein the phytochemical or bioactive compound is present in a concentration range of about 0.1 wt % to about 50 wt %; b) at least one lipid, wherein the lipid is present in a concentration range of about 5 wt % to about 90 wt %; c) at least one surfactant, wherein the surfactant is present in a concentration range of about 10 wt % to about 75 wt %; and d) at least one vitamin E, wherein the vitamin E is present in a concentration range of about 5 wt % to about 75 wt %.

In one embodiment, the nanoparticle is an antimicrobial nanoparticle.

In some embodiments, the at least one phytochemical or bioactive compound is selected from resveratrol, quercetin, curcumin, theaflavins, thearubigins, epigallocatechin gallate (EGCG), (+)-catechin, (−)-epicatechin, (−)-epicatechin gallate, (−)-epigallocatechin, (+)-gallocatechin, isorhamnetin, kaempferol, myricetin, apigenin, luteolin, baicalein, chrysin, forskolin, chlorophyll a, chlorophyll b, eriodictyol, hesperetin, naringenin, taxifolin, catechins, luteolin, cyanidin, genistein, daidzein, genistein, glycitein, biochanin A, formononetin, cyanidin, delphinidin, malvidin, pelargonidin, peonidin, petunidin, proanthocyanidins, α-carotene, β-carotene, β-cryptoxanthin, lutein, zeaxanthin, lycopene, or any combination thereof.

In one embodiment, the at least one phytochemical or bioactive compound is present in a concentration of about 10 wt %.

In some embodiments, the at least one lipid is selected from a fatty acid, wax, sterol, lipid-soluble vitamin, vitamin A, vitamin D, vitamin E, vitamin K, monoglyceride, diglyceride, triglyceride, phospholipid, L-α-phosphatidylcholine (PC), soy PC, egg PC, phosphatidic acid (PA), phosphatidylethanolamine (PE), lecithin, phosphatidylserine (PS), phosphoinositide, phosphatidylinositol (PI), phosphatidylinositol phosphate (PIP), phosphatidylinositol bisphosphate (PIP2), phosphatidylinositol trisphosphate (PIP3), phosphosphingolipid, sphingolipid, ceramide phosphorylcholine (SPH), ceramide phosphorylethanolamine (Cer-PE), ceramide phosphoryl lipid, or any combination thereof.

In one embodiment, the at least one lipid is present in a concentration of about 13 wt %. In one embodiment, the at least one lipid is present in a concentration of about 58 wt %.

In some embodiments, the at least one surfactant is selected from polyethylene glycol (PEG), functionalized PEG, polyethylene glycol 15-hydroxystearate, Tween 80, phospholipids, PEG40-stearate, PEG100-stearate, PEG (10-1000)-fatty acid, mustard, lecithin, soy lecithin, egg lecithin, monoglycerides, diglycerides, polysorbates, carrageenan, guar gum, canola oil, polysorbates (Tween™), sodium dodecyl sulfate, lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Byrj 52, Byrj S 100, Brij 721™, bile salts, sodium deoxycholate, sodium cholate, polyoxyl castor oil (Cremophor™), nonylphenol ethoxylate (Tergitol™), cyclodextrins, methylbenzethonium chloride (Hyamine™), or any combination thereof. In one embodiment, the at least one surfactant is polyethylene glycol 15-hydroxystearate.

In one embodiment, the at least one surfactant is in a concentration of about 40 wt %.

In some embodiments, the at least one vitamin E is selected from α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, δ-tocotrienol, δ-tocotrienol acetate, δ-tocotrienol nicotinate, or any combination thereof.

In one embodiment, the at least one vitamin E is in a concentration of about 40 wt %.

In one embodiment, the nanoparticle further comprises at least one coating agent. In some embodiments, the at least one coating agent is selected from a chitosan, starch, stabilizer, plasticizer, lipid, polysaccharide, protein, zein, soy protein, whey, casein, fatty acid, wax, neutral lipid, resin, cellulose, methyl cellulose, hydroxypropyl cellulose, hydroxypropyl methyl cellulose, alginate, or any combination thereof.

In some embodiments, the nanoparticle is coated with the at least one coating agent. In some embodiments, the nanoparticle is coated with chitosan, methyl cellulose, hydroxypropyl methyl cellulose, or any combination thereof.

In one embodiment, the nanoparticle further comprises at least one nutritional or bioactive agent.

In some embodiments, the nanoparticle reduces or inhibits the activity or level of at least one microorganism. In some embodiments, the microorganism is selected from bacterium, virus, pathogen, parasite, fungus, yeast, mold, or any combination thereof. In some embodiments, the bacterium is Escherichia, Escherichia coli, Salmonella enterica, Staphylococcus, Staphylococcus aureus, Listeria monocytogenes, Clostridium perfringens, Clostridium botulinum, Shigella boydii, Vibrio, Vibrio parahaemolyticus, Campylobacter, Campylobacter jejuni, Yersinia, Yersinia enterocolitica, Cronobacter sakazakii, Enterobacteriaceae, Erwinia herbicola, Rahnella aquatilis, Lacticaseibacillus casei, Leuconostoc mesenteroides, Bacillus cereus, Pseudomonadaceae, P. fluorescens, or any combination thereof.

In some embodiments, the yeast is selected from Candida sp., Candida pulcherrima, Candida humilis, Candida milleri, Candida tropicalis, Candida fermentati, Candida krusei, Candida parapsilosis, Candida guilliermondii, Candida lusitaniae, Candida rugosa, Cryptococcus sp., Rhodotorula sp., Trichosporon sp., Pichia sp., Torulaspora sp., C. lambica, C. sake, Debaryomyces polymorphus, or any combination thereof.

In some embodiments, the mold is selected from Botrytis, Botrytis cinerea, Rhizopus, Rhizopus stolonifer, Mucor, Mucor piriformis, Rhizoctonia solani, Phytophtora cactorum, Alternaria, Penicillium, Cladosporium, Aspergillus, Fusarium, Geotrichum, or any combination thereof.

In one embodiment, the nanoparticle is a biodegradable nanoparticle. In one embodiment, the nanoparticle is a biodegradable edible nanoparticle.

In some embodiments, the nanoparticle is a liposome, nanoemulsion, micelle, solid lipid nanoparticle (SLN), nanostructured lipid carrier (NLC), modified lipid nanoparticle, or any combination thereof.

In one embodiment, the nanoparticle preserves at least one nutrient. In some embodiments, the at least one nutrient is selected from vitamins (e.g., vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12), minerals (e.g., potassium, magnesium, calcium), antioxidant, or any combination thereof.

In one aspect, the present invention provides a composition comprising at least one nanoparticle of the present invention. In some embodiments, the composition is an edible coating, packing material, food preparation element, or any combination thereof.

In one embodiment, the composition preserves at least one nutrient.

In one aspect, the present invention provides a method of reducing or inhibiting the activity or level of at least one microorganism on a surface of an element.

In one aspect, the present invention provides a method of preventing the growth of at least one microorganism on a surface of an element.

In one aspect, the present invention provides a method of coating a surface of an element.

In some embodiments, the method comprises administering an effective amount of at least one nanoparticle of the present invention or a composition thereof to the surface of the element.

In some embodiments, the element is selected from the group consisting of food, packing material, food preparation element, and any combination thereof.

In one aspect, the present invention provides a method of increasing a shelf-life of food.

In one aspect, the present invention provides a method of preserving nutrients in food.

In one aspect, the present invention provides a method of improving nutritional value in food.

In some embodiments, the method comprises administering an effective amount of at least one nanoparticle of the present invention or a composition thereof to a surface of at least one selected from the group consisting of food, packing material, food preparation element, and any combination thereof.

In one aspect, the present invention provides a method of delivering at least one nutritional or bioactive agent to a subject.

In one aspect, the present invention provides a method of treating or preventing nutrient deficiency in a subject in need thereof.

In one aspect, the present invention provides a method of treating or preventing a disease, disorder, or condition in a subject in need thereof.

In some embodiments, the method comprises administering an effective amount of at least one nanoparticle of the present invention or a composition thereof to the subject.

In some embodiments, the disease, disorder, or condition is selected from obesity, diabetes, cancer, neurodegenerative disease, neurodegenerative disorder, disease associated with nutrient deficiency, disorder associated with nutrient deficiency, condition associated with nutrient deficiency, or any combination thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts representative transmission electron microscopy (TEM) image of epigallocatechin gallate-nanostructured lipid carriers (EGCG-NLCs) and epigallocatechin gallate-chitosan coated nanostructured lipid carriers (EGCG-CSNLCs) stained by 2% of uranyl acetate and epigallocatechin gallate (EGCG) detection and uptake. FIG. 1A depicts representative results demonstrating the detection of EGCG and other green tea catechins using a high-performance liquid chromatography (HPLC) system. FIG. 1B depicts representative results demonstrating the EGCG uptake by mouse macrophages. **, P<0.01 compared with native EGCG.

FIG. 2, comprising FIG. 2A through FIG. 2C, depicts representative stability results of nano-encapsulated and native EGCG in 1×PBS at pH 7.4 and at various temperatures. Three independent experiments were conducted at each time point. FIG. 2A depicts representative stability results of nano-encapsulated and native EGCG in 1× phosphate buffered saline (PBS) at pH 7.4 at 4° C. FIG. 2B depicts representative stability results of nano-encapsulated and native EGCG in 1×PBS at pH 7.4 at 22° C. FIG. 2C depicts representative stability results of nano-encapsulated and native EGCG in 1×PBS at pH 7.4 at 37° C.

FIG. 3 depicts representative changes of particle size, zeta potential, and PDI of Q-NLC at different temperatures.

FIG. 4, comprising FIG. 4A and FIG. 4F, depicts exemplary results demonstrating the stability of 200 μM Q and Q-NLCs in release medium. Values are expressed as means±S.D. *, p<0.05 as compared to V. NLC; #, p<0.05 as compared to Q; **, p<0.01; ***, p<0.001. Three independent experiments were conducted for this assay. FIG. 4A depicts representative results of remaining percentage of Q. FIG. 4B depicts representative results of in vitro hourly release profiles for native Q and Q-NLC. FIG. 4C depicts representative results demonstrating the viability of MCF-7 cells after treating different concentrations of Q, Q-NLCs, and V-NLCs for 48 hours. FIG. 4D depicts representative results demonstrating the viability of MDA-MB-231 cells after treating different concentrations of Q, Q-NLCs, and V-NLCs for 48 hours. FIG. 4E depicts representative results demonstrating the Q content in MCF-7 cells after treating them with different concentrations of Q and Q-NLC at 37° C. FIG. 4F depicts representative results demonstrating the Q content in MDA-MB-231 cells after treating them with different concentrations of Q and Q-NLC at 37° C.

FIG. 5 depicts representative results demonstrating the pharmacokinetics and bioavailability of Q after oral administration of free Q and Q-NLCs to rats.

FIG. 6 depicts exemplary photos to qualitatively compare the biocidal effectiveness of void nanoparticles (V-NPs), free (RES+EGCG+Q), and (RES+EGCG+Q)-NPs against Listeria monocytogenes.

FIG. 7 depicts representative results demonstrating anti-bacterial effects of 50 μg/mL of free R or R-NPs against bacteria (B), S. enteritidis (13076), L. monocytogenes (15313), E. coli (48953), and S. aureus (25923), after 24 hr.

FIG. 8 depicts representative results demonstrating anti-bacterial effects of 100 μg/mL of free R or R-NPs against bacteria (B), S. enteritidis (13076), L. monocytogenes (15313), E. coli (48953), and S. aureus (25923), after 6 and 24 hr.

FIG. 9 depicts representative results demonstrating anti-bacterial effects of 200 and 300 μg/mL of free R or R-NPs against bacteria (B), S. enteritidis (13076), L. monocytogenes (15313), E. coli (48953), and S. aureus (25923).

FIG. 10 depicts representative results demonstrating anti-bacterial effects of different concentrations of free R, free Q, R-NPs, and Q-NPs against S. enteritidis (13076), L. monocytogenes (15313), E. coli (48953), and S. aureus (25923).

FIG. 11 depicts representative results demonstrating anti-bacterial effects of different concentrations of free R and free Q vs. R-NPs and Q-NPs vs and RQ-NPs against S. enteritidis (13076), L. monocytogenes (15313), E. coli (48953), and S. aureus (25923).

FIG. 12 depicts representative results demonstrating anti-bacterial effects of CS, EGCG-CS, oligo-CS, gallic acid-CS (GA-CS) against S. enteritidis (13076), L. monocytogenes (15313), E. coli (48953), and S. aureus (25923) were investigated to determine, which should be used to coat with those NPs. The pH values were all adjusted to ˜5.3 in advance.

FIG. 13 depicts representative anti-bacterial results for R-NPs that were coated with 1 mg, 2 mg, and 4 mg CS for 30 mins and then CS-R-NPs diluted to 100 μg/mL based on the concentration of R-NPs in it.

FIG. 14 depicts representative anti-bacterial results for Q-NPs that were coated with 2 mg CS for 30 mins and then CS-Q-NPs diluted to 100 μg/mL based on the concentration of Q-NPs in it.

FIG. 15 depicts representative anti-bacterial results for RQ-NPs and CS-RQ-NPs. RQ-NPs containing 1 mg of R were coated with 2 mg CS for 30 mins. Then RQ-NPs and CS-RQ-NPs were diluted and used to treat bacteria at the concentration of 100 μg/mL of R in them.

FIG. 16 depicts representative illustration of NP structure.

FIG. 17, comprising FIG. 17A through FIG. 17C, depicts representative results demonstrating stability of NPs and in vivo release of R. Three independent experiments were conducted. Data are means±standard deviation (SD). Lines without sharing a common letter significantly differ at p<0.05. FIG. 17A depicts representative results demonstrating physical stability of various NPs. FIG. 17B depicts representative results demonstrating stability of R. FIG. 17C depicts representative in vitro release profiles of R.

FIG. 18 depicts representative results of qualitatively comparing photos to determine the antibacterial effect of free R and R-NPs at R concentration 100 μg/mL against S. enterica.

FIG. 19, comprising FIG. 19A through FIG. 19D, depicts representative results demonstrating antibacterial activities of R, Q, and RQ in free forms, NPs, and CS-coated NPs against S. enterica, E. coli, L. monocytogenes, or S. aureus. Control is the bacteria without treatments. Three independent experiments were conducted. Values are means±SD. Bars without sharing a common letter significantly differ at p<0.05. FIG. 19A depicts representative results demonstrating antibacterial activities of R, Q, and RQ in free forms, NPs, and CS-coated NPs against S. enterica. FIG. 19B depicts representative results demonstrating antibacterial activities of R, Q, and RQ in free forms, NPs, and CS-coated NPs against E. coli. FIG. 19C depicts representative results demonstrating antibacterial activities of R, Q, and RQ in free forms, NPs, and CS-coated NPs against L. monocytogenes. FIG. 19D depicts representative results demonstrating antibacterial activities of R, Q, and RQ in free forms, NPs, and CS-coated NPs against S. aureus.

FIG. 20, comprising FIG. 20A through FIG. 20C, depicts representative results demonstrating antibacterial mechanisms of various NPs against mRNA levels of sodA and oxyR in S. enterica, fnbB and sigB in S. aureus, and slp in E. coli. Three independent experiments were conducted. Bar length=500 nm. Values are means±SD. Bars without sharing a common letter significantly differ at p<0.05. FIG. 20A depicts representative results demonstrating antibacterial mechanisms of various NPs against mRNA levels of sodA and oxyR in S. enterica, fnbB and sigB in S. aureus, and slp in E. coli. FIG. 20A depicts representative results demonstrating antibacterial mechanisms of various NPs against mRNA levels of sodA and oxyR in S. enterica. FIG. 20B depicts representative results demonstrating antibacterial mechanisms of various NPs against mRNA levels of fnbB and sigB in S. aureus. FIG. 20C depicts representative results demonstrating antibacterial mechanisms of various NPs against mRNA levels of slp in E. coli.

FIG. 21 depicts representative results demonstrating the pharmacokinetics and bioavailability of free Q and Q-NPs after oral administration to rats.

### DETAILED DESCRIPTION

The present invention is based, in part, on the unexpected discovery that chitosan-coated nanoparticles, comprising resveratrol and quercetin, had synergistically increased antimicrobial activities, and enhanced nutrition value of foods by increasing phytochemical solubility and bioavailability. Thus, in one aspect, the present invention provides an antimicrobial nanoparticle comprising at least one phytochemical or bioactive compound, at least one lipid, at least one surfactant, and at least one vitamin E. In one aspect, the present invention provides an antimicrobial composition comprising at least one phytochemical or bioactive compound, at least one lipid, at least one surfactant, and at least one vitamin E. In some embodiments, the nanoparticle or composition further comprises at least one agent (e.g., coating agent, protein, nutritional or bioactive agent, etc.).

In some aspects, the present invention also provides a composition comprising at least one nanoparticle of the present invention. In some embodiments, the composition is an edible coating. Thus, in some aspects, the present invention provides methods of reducing or inhibiting the activity or level of at least one microorganism on a surface of an element (e.g., food). In other aspects, the present invention provides methods of preventing the growth of at least one microorganism on a surface of an element (e.g., food). In other aspects, the present invention provides methods of increasing a shelf-life of food. In another aspect, the present invention provides a method of maintaining nutrients in food. In another aspect, the present invention provides a method of increasing or improving nutritional value in food. In other aspects, the present invention provides methods of delivering at least one nutritional or bioactive agent (e.g., vitamins, minerals, etc.). Thus, in some aspects, the present invention provides a method of treating or preventing nutrient deficiency in a subject. In other aspects, the present invention provides a method of treating or preventing a disease, disorder, or condition (e.g., obesity, diabetes, cancer, neurodegenerative disease, neurodegenerative disorder, disease associated with nutrient deficiency, disorder associated with nutrient deficiency, condition associated with nutrient deficiency, or any combination thereof).

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

As used herein, the term “nanoparticle” refers to particles having a particle size on the nanometer scale (e.g., about 1 nm-10,000 nm). For example, the nanoparticle may have a particle size up to about 2,000 nm. In another example, the nanoparticle may have a particle size up to about 100 nm. In another example, the nanoparticle may have a particle size up to about 6 nm. As used herein, “nanoparticle” refers to a number of nanoparticles, including, but not limited to, liposomes, lipid nanoparticles, polymer nanoparticles, organic nanoparticles, inorganic nanoparticles, biocompatible nanoparticles, such as biocompatible organic nanoparticles, biocompatible inorganic nanoparticles, etc., nanoclusters, nanocapsules, core-shell nanocapsules, nanovesicles, micelles, block copolymer micelles, lamaellae shaped particles, polymersomes, dendrimers, emulsions, exosomes, self-emulsifying drug delivery systems (SEDDS), microspheres, micro-structured lipid carriers, nano-structured lipid carriers, and other nano-size particles of various other small fabrications that are known to those of skill in the art. Examples of suitable nanoparticles useful in the invention include, but are not limited to, those described in Wang et al., 2014, J. Nutri. Biochem., 25:363-376, which is incorporated herein by reference in its entirety. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

As used herein, the phrase “lipid nanoparticle” refers to a transfer vehicle comprising one or more lipids (e.g., cationic lipids, non-cationic lipids, polar lipids, and PEG-modified lipids) and/or one or more polymers. Examples of suitable lipids include, but are not limited to, the phospholipid compounds (e.g., phosphatidylglycerol, phosphatidylcholine, phosphatidylserine, phosphatidylethanolamine, sphingolipids, cerebrosides, and gangliosides). Examples of suitable polymers include, but are not limited to, polyacrylates, polyalkycyanoacrylates, polylactide, polylactide-polyglycolide copolymers, polycaprolactones, dextran, albumin, gelatin, alginate, collagen, chitosan, cyclodextrins, dendrimers, and polyethylenimine.

The term “liposome” as used herein refers to microscopic vesicles or particles made up of one or more lipid bilayers enclosing an internal aqueous medium. To form liposomes, the presence of at least one “vesicle-forming lipid” is needed, which is an amphipathic lipid capable of either forming or being incorporated into a lipid bilayer. Any suitable vesicle-forming lipid may be used to form the lipid bilayer constituting the liposomes. Vesicle-forming lipid includes, but not limited to, phospholipids such as phosphatidylcholine (PC), phosphatidylglycerol (PG), phosphatidylinositol (PI), phosphatidic acid (PA), phosphatidylethanolamine (PE) or phosphatidylserine (PS), and charged lipids, such as a positively charge lipid or a negatively charged lipid. The term “liposome” may have the same meaning and may be interchanged with the term “lipid vesicle”. Liposome examples include large unilamellar vesicles, multilamellar vesicles, paucilamellar vesicles, small unilamellar vesicles, reverse phase evaporation vesicles, French press vesicles, and ether injection vesicles. Products incorporating liposomes include, but are not limited to, adjuvants, drug carriers, and cleansers. The following references disclose non-limiting examples of methods and/or non-limiting examples of apparatuses for manufacturing liposomes: “LIPOSOMES—Potential for Commercial Application”, by Dr. Norman D. Weiner, presented at the Emulsion-Suspension Technology Conference, Oct. 20-23, 1997, at New Brunswick, N.J.; U.S. Pat. No. 4,911,928 to Wallach, issued Mar. 27, 1990; U.S. Pat. No. 4,855,090 to Wallach, issued Aug. 8, 1989; and U.S. Pat. No. 4,895,452 to Yiournas et al., issued Jan. 23, 1990.

For example, the term “nanocapsule” refers to a vesicular system or hollow particle with a shell surrounding a core-forming space, which, in certain instances, can be used for transporting a payload on a nanoscale level. A nanocapsule may also be a nano-sized version of a container. The payload of the nanocapsule can be, but is not limited to drugs, medicaments, pharmaceutical compositions, chemical compositions, therapeutic compositions, biological macromolecules, dyes, biological material, immunological compositions, nutritional compositions, vitamins, proteins, nucleic acids, antibodies, and vaccines. Various materials may be used for producing such nanocapsules. Nanocapsule refers to a particle having a hollow core that is surrounded by a shell, such that the particle has a size of less than about 1000 nanometers. When a nanocapsule includes a bioactive component, the bioactive component is located in the core that is surrounded by the shell of the nanocapsule.

As used herein, the term “nanocage” refers to a nanocapsule, whereby the shell is not solid, as described for the nanocapsule, but has multiple holes or pores in its shell, thereby making it possible for the payload within the core of the nanocage to come into contact with the surrounding environment. These holes or pores may be regular or irregular in shape and/or spacing on the surface of the particle.

The term “micelle”, a useful article in the employment of a general aspect of the present invention, can generally be thought of as a small-on the order of usually nanometers in diameter-aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

The term “polymersome” as used herein refers to a vesicle-type which is typically composed of block copolymer amphiphiles, i.e., synthetic amphiphiles that have an amphiphilicity similar to that of lipids. By virtue of their amphiphilic nature (having a more hydrophilic block (head) and a more hydrophobic block (tail)), the block copolymers are capable of self-assembling into a head-to-tail and tail-to-head bilayer structure similar to liposomes. Compared to liposomes, polymersomes have much larger molecular weights, with number average molecular weights typically ranging from 1,000 to 100,000, from 2,500 to 50,000 or from 5,000 to 25,000, are typically chemically more stable, less leaky, less prone to interfere with biological membranes, and less dynamic due to a lower critical aggregation concentration. These properties result in less opsonisation and longer circulation times. The terms “more hydrophilic” and “more hydrophobic” as used in the context of the ampohiphilic nature of the block copolymers are used in a relative sense. i.e., both can be either hydrophilic or hydrophobic, as long as the difference in polarity between the blocks is sufficient for the formation of polymersomes according to the present invention. In view of the creation of a cavity in which water may be incorporated, in certain aspects the more hydrophilic end of the polymer is to be hydrophilic per se. Further, in view of the use as a therapeutic agent carrier, it is desired that also hydrophobic and/or hydrophilic therapeutic agents can be incorporated into the polymersomes. In one embodiment, the hydrophobic end of the polymer is hydrophobic per se. In one embodiment, the amphiphilic nature of the block copolymers is realized in the form of a block copolymer comprising a block made up of more hydrophilic monomeric units (A) and a block made up of more hydrophobic units (B), the block copolymer having the general structure AnBm, with n and m being integers of from 5 to 5,000, 10 to 1,000, or 10 to 500. It is also conceivable that one or more further units or blocks are built-in, e.g., a unit C with an intermediate hydrophilicity so as to yield a terpolymer having the general structure AnCpBm, with n and m being as defined above, and p being an integer of from 5 to 5,000, 10 to 1,000, or 10 to 500. Any of the blocks can itself be a copolymer, i.e., comprise different monomeric units of the required hydrophilic respectively hydrophobic nature. In one embodiment, the blocks themselves are homopolymeric. Any of the blocks, in particular the more hydrophilic block, may bear charges. The number and type of charges may depend on the pH of the environment. Any combination of positive and/or negative charges on any of the blocks is contemplated by the present invention.

“Dendrimers” have descriptions, definitions and understandings in the literature. For example, and without limitation and including other and similar definitions, descriptions and understandings in the art, the term dendrimer from the Greek word, “dendron”, for tree, can refer to a synthetic, three-dimensional molecule with branching parts. Descriptions and understandings of dendrimers can be gleaned from Holister et al., Dendrimers, Technology White Papers nr. 6, pub. October 2003 by cientifica, as well as the other literature published by those skilled in the art on dendrimers, all of which are incorporated herein by reference.

“Lamella” is a term whose definitions, descriptions and understandings are also known to those of skill in the art and which are incorporated herein by reference. In a very general sense, lamella or lamellae refers to plate-like, gill-shaped or other layered structures.

The definitions, descriptions and understandings of “nanovesicle” are well known to those of skill in the art, and are incorporated herein by reference. For example, “nanovesicle” can refer to a variety of small sac, sac-like or globular structures capable of containing fluid or other material therein.

As used herein, the term “exosome” refers to a subset of circulating microvesicles that are preformed microvesicles that are released from the cell following the exocytic fusion of intracellular multivesicular bodies with the plasma membrane, i.e., exosomes have an endocytic origin. As used herein, it is not intended that an exosome of the invention be limited by any particular size or size range. For example, exosome include a variety of nanoparticles, including microvesicles, epididimosomes, argosomes, exosome-like vesicles, microparticles, promininosomes, prostasomes, dexosomes, texosomes, dex, tex, archeosomes and oncosomes. Exosomes are secreted by a wide range of cells, such as mammalian cells, and are secreted under both normal and pathological conditions. Exosomes, in some embodiments, function as intracellular messengers by virtue of carrying mRNA or other contents from a first cell to another cell (or plurality of cells). In some embodiments, exosomes are involved in blood coagulation, immune modulation, metabolic regulation, cell division, and other cellular processes.

The term “emulsion” as used herein refers to a mixture of two or more substances, such as liquids, that are normally immiscible, in which one substance forms droplets that are dispersed within another substance. One substance (the dispersed phase) is dispersed in the other (the continuous phase). Depending on the substances used, the droplets of an emulsion may be in the range of 1 nm to 100 μm, e.g., 1 nm to 100 nm, 1 μm to 50 μm, etc. For example, in one embodiment, the continuous phase is an aqueous phase and the dispersed phase is an organic (oily or hydrophobic) phase; that is, the emulsion is an oil-in-water emulsion.

As used herein, the term “self-emulsifying drug delivery systems (SEDDS)” refers to isotropic mixtures, consisting of oils, surfactants, and/or cosolvents. In some embodiments, SEDDS increase solubility and bioavailability of poorly soluble drugs. For example, in some embodiments, designed SEDDS formulations are used to improve the oral absorption of highly lipophilic compounds. Examples of suitable SEDDS useful in the invention include, but are not limited to, those described in WO 2002/007712 A2 and Wang et al., 2014, J. Nutri. Biochem., 25:363-376, which are incorporated herein by reference in their entireties.

As used herein, the term “polymorph” refers to crystalline forms having the same chemical composition but different spatial arrangements of the molecules, atoms, and/or ions forming the crystal.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid, and any combinations or derivatives thereof. Other examples include, but are not limited to, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya, and biosynthetic gum. Other examples of useful primary particle and/or secondary stabilizers include polymers, such as polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

The terms “coat,” “coated,” or “coating,” as used herein, refer to at least a partial coating of the nanoparticle. One hundred percent coverage is not necessarily implied by these terms.

An “effective amount” or “therapeutically effective amount”, as used herein, means an amount, which provides a therapeutic or prophylactic benefit. For example, an “effective amount” or “therapeutically effective amount” of a nanoparticle is that amount of compound, which is sufficient to provide a beneficial effect (e.g., deliver a nutritional or bioactive agent) to the subject to which the nanoparticle is administered.

“Pharmaceutically acceptable” refers to those properties and/or substances, which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, anti-bacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. In some embodiments, such salts are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts, such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts, such as, for example, sodium, potassium, calcium and ammonium salts, and organic alkali salts, such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional or bioactive agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium, or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional or bioactive agents, and the like. The active agents described herein may be found in their native state, however, they will generally be provided in the form of a salt. The active agents described herein include their isomers, analogs, and derivatives.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the unexpected discovery that chitosan-coated nanoparticles, comprising resveratrol and quercetin, had synergistically increased antimicrobial activities that enhanced nutrition value of foods by increasing phytochemical bioavailability. Thus, in one aspect, the present invention provides an antimicrobial nanoparticle comprising at least one phytochemical or bioactive compound, at least one lipid, at least one surfactant, and at least one vitamin E. In another aspect, the present invention provides an antimicrobial composition comprising at least one phytochemical or bioactive compound, at least one lipid, at least one surfactant, and at least one vitamin E. In some embodiments, the nanoparticle or composition further comprises at least one agent (e.g., coating agent, protein, nutritional or bioactive agent, etc.).

In some aspects, the present invention also provides a composition comprising at least one nanoparticle or composition of the present invention. In some embodiments, the composition is an edible coating. Thus, in some aspects, the present invention provides methods of reducing or inhibiting the activity or level of at least one microorganism on a surface of an element (e.g., food). In other aspects, the present invention provides methods of preventing the growth of at least one microorganism on a surface of an element (e.g., food). In other aspects, the present invention provides methods of increasing a shelf-life of food. In another aspect, the present invention provides a method of maintaining nutrients in food. In another aspect, the present invention provides a method of increasing or improving nutritional value in food.

In other aspects, the present invention provides methods of delivering at least one nutritional or bioactive agent (e.g., vitamins, minerals, etc.). Thus, in some aspects, the present invention provides a method of treating or preventing nutrient deficiency in a subject. In other aspects, the present invention provides a method of treating or preventing a disease, disorder, or condition (e.g., obesity, diabetes, cancer, neurodegenerative disease, neurodegenerative disorder, disease associated with nutrient deficiency, disorder associated with nutrient deficiency, condition associated with nutrient deficiency, or any combination thereof).

### Compositions and Nanoparticles

The present invention relates, in part, to a nanoparticle comprising at least one phytochemical or bioactive compound, at least one lipid, at least one surfactant and at least one vitamin E. In various embodiments, the nanoparticle reduces the activity or level of at least one microorganism. In some embodiments, the nanoparticle inhibits the activity of at least one microorganism. In some embodiments, the nanoparticle inhibits or reduces the growth of at least one microorganism. Examples of such microorganism include, but are not limited to, a bacterium, virus, pathogen, parasite, fungus, yeast, mold, or any combination thereof. In some embodiments, the nanoparticle preserves at least one nutrient. Examples of such nutrients include, but are not limited to, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, or any combination thereof.

Thus, in various embodiments, the nanoparticle is an antimicrobial nanoparticle. In some embodiments, the antimicrobial nanoparticle is an antibacterial nanoparticle, antiviral nanoparticle, antiparasitic nanoparticle, antipathogenic nanoparticle, antifungal nanoparticle, or any combination thereof.

In another aspect, the present invention relates, in part, to a composition comprising at least one phytochemical or bioactive compound, at least one lipid, at least one surfactant and at least one vitamin E. In various embodiments, the composition reduces the activity or level of at least one microorganism. In some embodiments, the composition inhibits the activity of at least one microorganism. In some embodiments, the composition inhibits or reduces the growth of at least one microorganism. Examples of such microorganism include, but are not limited to, a bacterium, virus, pathogen, parasite, fungus, yeast, mold, or any combination thereof. In some embodiments, the composition preserves at least one nutrient. Examples of such nutrients include, but are not limited to, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, or any combination thereof.

Thus, in various embodiments, the composition is an antimicrobial composition. In some embodiments, the antimicrobial composition is an antibacterial composition, antiviral composition, antiparasitic composition, antipathogenic composition, antifungal composition, or any combination thereof.

In various embodiments, the phytochemical or bioactive compound is encapsulated within the nanoparticle, adhered to the surface of the nanoparticle, integrated into the structure of the nanoparticle, bound to the nanoparticle, or any combination thereof. In some embodiments, the phytochemical or bioactive compounds is a carotenoid compound, polyphenol compound, or a combination thereof. In some embodiments, the polyphenol compound is a phenolic acid, flavonoid, stilbene, lignan, or any combination thereof. In some embodiments, the flavonoid is anthocyanin, flavone, flavanone, isoflavone, flavonol, flavanol, or any combination thereof. In some embodiments, the flavanol is catechin, epicatechin, or any combination thereof. Examples of such phytochemical or bioactive compound include, but are not limited to, resveratrol, quercetin, curcumin, theaflavins, thearubigins, epigallocatechin gallate (EGCG), (+)-catechin, (−)-epicatechin, (−)-epicatechin gallate, (−)-epigallocatechin, (+)-gallocatechin, isorhamnetin, kaempferol, myricetin, apigenin, luteolin, baicalein, chrysin, forskolin, chlorophyll a, chlorophyll b, eriodictyol, hesperetin, naringenin, taxifolin, catechins, luteolin, cyanidin, genistein, daidzein, genistein, glycitein, biochanin A, formononetin, cyanidin, delphinidin, malvidin, pelargonidin, peonidin, petunidin, proanthocyanidins, α-carotene, β-carotene, β-cryptoxanthin, lutein, zeaxanthin, lycopene, or any combination thereof.

In various embodiments, the nanoparticle or composition comprises the phytochemical compound in a concentration range of about 0.01 wt % to about 99.99 wt %. In some embodiments, the nanoparticle or composition comprises the phytochemical compound in a concentration range of about 0.1 wt % to about 99.9 wt %. In some embodiments, the nanoparticle or composition comprises the phytochemical compound in a concentration range of about 1 wt % to about 70 wt %. In some embodiments, the nanoparticle or composition comprises the phytochemical compound in a concentration range of about 1 wt % to about 60 wt %. In some embodiments, the nanoparticle or composition comprises the phytochemical compound in a concentration range of about 1 wt % to about 55 wt %. In some embodiments, the nanoparticle or composition comprises the phytochemical compound in a concentration range of about 0.1 wt % to about 50 wt %. In some embodiments, the nanoparticle or composition comprises the phytochemical compound in a concentration range of about 1 wt % to about 50 wt %. In some embodiments, the nanoparticle or composition comprises the phytochemical compound in a concentration range of about 1 wt % to about 10 wt %.

For example, in some embodiments, the nanoparticle or composition comprises the phytochemical compound in a concentration of about 0.01 wt %. In some embodiments, the nanoparticle or composition comprises the phytochemical compound in a concentration of about 0.02 wt %. In some embodiments, the nanoparticle or composition comprises the phytochemical compound in a concentration of about 0.05 wt %. In some embodiments, the nanoparticle or composition comprises the phytochemical compound in a concentration of about 0.15 wt %. In some embodiments, the nanoparticle or composition comprises the phytochemical compound in a concentration of about 1.0 wt %. In some embodiments, the nanoparticle or composition comprises the phytochemical compound in a concentration of about 7 wt %. In some embodiments, the nanoparticle or composition comprises the phytochemical compound in a concentration of about 10 wt %.

In various embodiments, the nanoparticle or composition comprises the bioactive compound in a concentration range of about 0.01 wt % to about 99.99 wt %. In some embodiments, the nanoparticle or composition comprises the bioactive compound in a concentration range of about 0.1 wt % to about 99.9 wt %. In some embodiments, the nanoparticle or composition comprises the bioactive compound in a concentration range of about 1 wt % to about 70 wt %. In some embodiments, the nanoparticle or composition comprises the bioactive compound in a concentration range of about 1 wt % to about 60 wt %. In some embodiments, the nanoparticle or composition comprises the bioactive compound in a concentration range of about 1 wt % to about 55 wt %. In some embodiments, the nanoparticle or composition comprises the bioactive compound in a concentration range of about 0.1 wt % to about 50 wt %. In some embodiments, the nanoparticle or composition comprises the bioactive compound in a concentration range of about 1 wt % to about 50 wt %. In some embodiments, the nanoparticle or composition comprises the bioactive compound in a concentration range of about 1 wt % to about 10 wt %.

For example, in some embodiments, the nanoparticle or composition comprises the bioactive compound in a concentration of about 0.01 wt %. In some embodiments, the nanoparticle or composition comprises the bioactive compound in a concentration of about 0.02 wt %. In some embodiments, the nanoparticle or composition comprises the bioactive compound in a concentration of about 0.05 wt %. In some embodiments, the nanoparticle or composition comprises the bioactive compound in a concentration of about 0.15 wt %. In some embodiments, the nanoparticle or composition comprises the bioactive compound in a concentration of about 1.0 wt %. In some embodiments, the nanoparticle or composition comprises the bioactive compound in a concentration of about 7 wt %. In some embodiments, the nanoparticle or composition comprises the bioactive compound in a concentration of about 10 wt %.

In some embodiments, the lipid is selected from a fatty acid, wax, sterol, lipid-soluble vitamin, such as vitamins A, D, E, and K, monoglyceride, diglyceride, triglyceride, phospholipids, cholesterol, PEG-phospholipids, PEG-fatty acids, lipid conjugates, or any combination thereof. In some embodiments, the phospholipid is selected from phosphatidylcholine (PC), such as soy or egg L-α-phosphatidylcholine (PC), phosphatidic acid (phosphatidate) (PA), phosphatidylethanolamine (cephalin) (PE), lecithin, phosphatidylserine (PS), phosphoinositide, phosphatidylinositol (PI), phosphatidylinositol phosphate (PIP), phosphatidylinositol bisphosphate (PIP2), phosphatidylinositol trisphosphate (PIP3), phosphosphingolipid, phospholipid-phosphatic acid, sphingolipid, ceramide phosphorylcholine (sphingomyelin) (SPH), ceramide phosphorylethanolamine (sphingomyelin) (Cer-PE), ceramide phosphoryl lipid, or any combination thereof.

In various embodiments, the nanoparticle or composition comprises one or more lipids in a concentration range of about 0.1 wt % to about 100 wt %. In some embodiments, the nanoparticle or composition comprises one or more lipids in a concentration range of about 1 wt % to about 100 wt %. In some embodiments, the nanoparticle or composition comprises one or more lipids in a concentration range of about 5 wt % to about 90 wt %. In some embodiments, the nanoparticle or composition comprises one or more lipids in a concentration range of about 1 wt % to about 70 wt %. In some embodiments, the nanoparticle or composition comprises one or more lipids in a concentration range of about 10 wt % to about 80 wt %. In some embodiments, the nanoparticle or composition comprises one or more lipids in a concentration range of about 10 wt % to about 70 wt %. In some embodiments, the nanoparticle or composition comprises one or more lipids in a concentration range of about 10 wt % to about 50 wt %. In some embodiments, the nanoparticle or composition comprises one or more lipids in a concentration range of about 15 wt % to about 45 wt %. In some embodiments, the nanoparticle or composition comprises one or more lipids in a concentration range of about 35 wt % to about 40 wt %.

For example, in some embodiments, the nanoparticle or composition comprises one or more lipids in a concentration of about 1 wt %. In some embodiments, the nanoparticle or composition comprises one or more lipids in a concentration of about 2 wt %. In some embodiments, the nanoparticle or composition comprises one or more lipids in a concentration of about 5 wt %. In some embodiments, the nanoparticle or composition comprises one or more lipids in a concentration of about 5.5 wt %. In some embodiments, the nanoparticle or composition comprises one or more lipids in a concentration of about 10 wt %. In some embodiments, the nanoparticle or composition comprises one or more lipids in a concentration of about 13 wt %. In some embodiments, the nanoparticle or composition comprises one or more lipids in a concentration of about 15 wt %. In some embodiments, the nanoparticle or composition comprises one or more lipids in a concentration of about 20 wt %. In some embodiments, the nanoparticle or composition comprises one or more lipids in a concentration of about 25 wt %. In some embodiments, the nanoparticle or composition comprises one or more lipids in a concentration of about 30 wt %. In some embodiments, the nanoparticle or composition comprises one or more lipids in a concentration of about 35 wt %. In some embodiments, the nanoparticle or composition comprises one or more lipids in a concentration of about 37 wt %. In some embodiments, the nanoparticle or composition comprises one or more lipids in a concentration of about 40 wt %. In some embodiments, the nanoparticle or composition comprises one or more lipids in a concentration of about 45 wt %. In some embodiments, the nanoparticle or composition comprises one or more lipids in a concentration of about 50 wt %. In some embodiments, the nanoparticle or composition comprises one or more lipids in a concentration of about 58 wt %. In some embodiments, the nanoparticle or composition comprises one or more lipids in a concentration of about 60 wt %. In some embodiments, the nanoparticle or composition comprises one or more lipids in a concentration of about 70 wt %. In some embodiments, the nanoparticle or composition comprises one or more lipids in a concentration of about 80 wt %. In some embodiments, the nanoparticle or composition comprises one or more lipids in a concentration of about 90 wt %. In some embodiments, the nanoparticle or composition comprises one or more lipids in a concentration of about 95 wt %. In some embodiments, the nanoparticle or composition comprises one or more lipids in a concentration of about 95.5 wt %. In some embodiments, the nanoparticle or composition comprises one or more lipids in a concentration of about 99 wt %. In some embodiments, the nanoparticle or composition comprises one or more lipids in a concentration of about 99.9 wt %. In some embodiments, the nanoparticle or composition comprises one or more lipids in a concentration of about 100 wt %.

In some embodiments, the surfactant is any surfactant that is known to those of skill in the art, any emulsifier that is known to those of skill in the art, or any combination thereof. Examples of such surfactant include, but are not limited to, polyethylene glycol (PEG), functionalized PEG, such as polyethylene glycol 15-hydroxystearate, Tween 80, phospholipids, PEG40-stearate, PEG100-stearate, PEG (10-1000)-fatty acid, mustard, lecithin, such as soy lecithin and egg lecithin, monoglycerides, diglycerides, polysorbates, carrageenan, guar gum, canola oil, polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Brij 721™, bile salts, such as sodium deoxycholate and sodium cholate, polyoxyl castor oil (Cremophor™), nonylphenol ethoxylate (Tergitol™), cyclodextrins, methylbenzethonium chloride (Hyamine™), or a combination thereof. Examples of such emulsifier include, but are not limited to emulsifiers listed in Emulsifiers: Types and Uses, R Miller, Kansas State University, Manhattan, KS, USA, 2016 Elsevier Ltd. All rights reserved, incorporated herein by reference in its entirety.

In various embodiments, the nanoparticle or composition comprises one or more surfactants in a concentration range of about 0.1 wt % to about 100 wt %. In some embodiments, the nanoparticle or composition comprises one or more surfactants in a concentration range of about 1 wt % to about 100 wt %. In some embodiments, the nanoparticle or composition comprises one or more surfactants in a concentration range of about 1 wt % to about 70 wt %. In some embodiments, the nanoparticle or composition comprises one or more surfactants in a concentration range of about 10 wt % to about 80 wt %. In some embodiments, the nanoparticle or composition comprises one or more surfactants in a concentration range of about 10 wt % to about 75 wt %. In some embodiments, the nanoparticle or composition comprises one or more surfactants in a concentration range of about 10 wt % to about 70 wt %. In some embodiments, the nanoparticle or composition comprises one or more surfactants in a concentration range of about 10 wt % to about 50 wt %. In some embodiments, the nanoparticle or composition comprises one or more surfactants in a concentration range of about 15 wt % to about 45 wt %. In some embodiments, the nanoparticle or composition comprises one or more surfactants in a concentration range of about 35 wt % to about 40 wt %.

For example, in some embodiments, the nanoparticle or composition comprises one or more surfactants in a concentration of about 1 wt %. In some embodiments, the nanoparticle or composition comprises one or more surfactants in a concentration of about 2 wt %. In some embodiments, the nanoparticle or composition comprises one or more surfactants in a concentration of about 5 wt %. In some embodiments, the nanoparticle or composition comprises one or more surfactants in a concentration of about 5.5 wt %. In some embodiments, the nanoparticle or composition comprises one or more surfactants in a concentration of about 10 wt %. In some embodiments, the nanoparticle or composition comprises one or more surfactants in a concentration of about 13 wt %. In some embodiments, the nanoparticle or composition comprises one or more surfactants in a concentration of about 15 wt %. In some embodiments, the nanoparticle or composition comprises one or more surfactants in a concentration of about 20 wt %. In some embodiments, the nanoparticle or composition comprises one or more surfactants in a concentration of about 25 wt %. In some embodiments, the nanoparticle or composition comprises one or more surfactants in a concentration of about 30 wt %. In some embodiments, the nanoparticle or composition comprises one or more surfactants in a concentration of about 35 wt %. In some embodiments, the nanoparticle or composition comprises one or more surfactants in a concentration of about 37 wt %. In some embodiments, the nanoparticle or composition comprises one or more surfactants in a concentration of about 40 wt %. In some embodiments, the nanoparticle or composition comprises one or more surfactants in a concentration of about 45 wt %. In some embodiments, the nanoparticle or composition comprises one or more surfactants in a concentration of about 50 wt %. In some embodiments, the nanoparticle or composition comprises one or more surfactants in a concentration of about 60 wt %. In some embodiments, the nanoparticle or composition comprises one or more surfactants in a concentration of about 70 wt %. In some embodiments, the nanoparticle or composition comprises one or more surfactants in a concentration of about 80 wt %. In some embodiments, the nanoparticle or composition comprises one or more surfactants in a concentration of about 90 wt %. In some embodiments, the nanoparticle or composition comprises one or more surfactants in a concentration of about 95 wt %. In some embodiments, the nanoparticle or composition comprises one or more surfactants in a concentration of about 95.5 wt %. In some embodiments, the nanoparticle or composition comprises one or more surfactants in a concentration of about 99 wt %. In some embodiments, the nanoparticle or composition comprises one or more surfactants in a concentration of about 99.9 wt %. In some embodiments, the nanoparticle or composition comprises one or more surfactants in a concentration of about 100 wt %.

In some embodiments, the vitamin E is α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, δ-tocotrienol, δ-tocotrienol acetate, δ-tocotrienol nicotinate, or any combination thereof.

In various embodiments, the nanoparticle or composition comprises one or more vitamin E in a concentration range of about 0.1 wt % to about 100 wt %. In some embodiments, the nanoparticle or composition comprises one or more vitamins E in a concentration range of about 1 wt % to about 100 wt %. In some embodiments, the nanoparticle or composition comprises one or more vitamins E in a concentration range of about 1 wt % to about 70 wt %. In some embodiments, the nanoparticle or composition comprises one or more vitamins E in a concentration range of about 5 wt % to about 75 wt %. In some embodiments, the nanoparticle or composition comprises one or more vitamins E in a concentration range of about 10 wt % to about 80 wt %. In some embodiments, the nanoparticle or composition comprises one or more vitamins E in a concentration range of about 10 wt % to about 70 wt %. In some embodiments, the nanoparticle or composition comprises one or more vitamins E in a concentration range of about 10 wt % to about 50 wt %. In some embodiments, the nanoparticle or composition comprises one or more vitamins E in a concentration range of about 15 wt % to about 45 wt %. In some embodiments, the nanoparticle or composition comprises one or more vitamins E in a concentration range of about 35 wt % to about 40 wt %.

For example, in some embodiments, the nanoparticle or composition comprises one or more vitamins E in a concentration of about 1 wt %. In some embodiments, the nanoparticle or composition comprises one or more vitamins E in a concentration of about 2 wt %. In some embodiments, the nanoparticle or composition comprises one or more vitamins E in a concentration of about 5 wt %. In some embodiments, the nanoparticle or composition comprises one or more vitamins E in a concentration of about 5.5 wt %. In some embodiments, the nanoparticle or composition comprises one or more vitamins E in a concentration of about 10 wt %. In some embodiments, the nanoparticle or composition comprises one or more vitamins E in a concentration of about 13 wt %. In some embodiments, the nanoparticle or composition comprises one or more vitamins E in a concentration of about 15 wt %. In some embodiments, the nanoparticle or composition comprises one or more vitamins E in a concentration of about 20 wt %. In some embodiments, the nanoparticle or composition comprises one or more vitamins E in a concentration of about 25 wt %. In some embodiments, the nanoparticle or composition comprises one or more vitamins E in a concentration of about 30 wt %. In some embodiments, the nanoparticle or composition comprises one or more vitamins E in a concentration of about 35 wt %. In some embodiments, the nanoparticle or composition comprises one or more vitamins E in a concentration of about 37 wt %. In some embodiments, the nanoparticle or composition comprises one or more vitamins E in a concentration of about 40 wt %. In some embodiments, the nanoparticle or composition comprises one or more vitamins E in a concentration of about 45 wt %. In some embodiments, the nanoparticle or composition comprises one or more vitamins E in a concentration of about 50 wt %. In some embodiments, the nanoparticle or composition comprises one or more vitamins E in a concentration of about 60 wt %. In some embodiments, the nanoparticle or composition comprises one or more vitamins E in a concentration of about 70 wt %. In some embodiments, the nanoparticle or composition comprises one or more vitamins E in a concentration of about 80 wt %. In some embodiments, the nanoparticle or composition comprises one or more vitamins E in a concentration of about 90 wt %. In some embodiments, the nanoparticle or composition comprises one or more vitamins E in a concentration of about 95 wt %. In some embodiments, the nanoparticle or composition comprises one or more vitamins E in a concentration of about 95.5 wt %. In some embodiments, the nanoparticle or composition comprises one or more vitamins E in a concentration of about 99 wt %. In some embodiments, the nanoparticle or composition comprises one or more vitamins E in a concentration of about 99.9 wt %. In some embodiments, the nanoparticle or composition comprises one or more vitamins E in a concentration of about 100 wt %.

In various embodiments, the nanoparticle or composition further comprises at least one coating agent. In one embodiment, the coating agent coats the outside surface of the nanoparticle. In one embodiment, the coating agent coats a portion of the outside surface of the nanoparticle. In some embodiments, the coating agent comprises a chitosan, starch, stabilizer, plasticizer, lipid, polysaccharide, protein, zein, soy protein, whey, casein, fatty acid, wax, neutral lipid, resin, cellulose, methyl cellulose, hydroxypropyl cellulose, hydroxypropyl methyl cellulose, alginate, or any combination thereof. For example, in some embodiments, the nanoparticle is coated with chitosan, methyl cellulose, hydroxypropyl methyl cellulose, or any combination thereof.

In one embodiment, the coating agent comprises a biocompatible polymer.

Examples of coating agents include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, phospholipids, albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(3-hydroxyvalerate), poly(D,L-lactide-co-glycolide), poly(l-lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, or any combination thereof.

In some embodiments, the coating agent comprises one or more molecules selected from the group consisting of gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, and any combination thereof.

In one embodiment, the coating agent comprises chitosan. In one embodiment, the chitosan coats the outside surface of the nanoparticle.

In various embodiments, the nanoparticle or composition further comprises at least one nutritional or bioactive agent. In some embodiments, the nutritional or bioactive agent comprises a vitamin, mineral, or any combination thereof. In some embodiments, the nutritional or bioactive agent comprises a vitamin (e.g., vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, vitamin C, vitamin D, vitamin E, vitamin K), minerals (e.g., potassium, magnesium, calcium), niacin, pantothenic acid, biotin, coline, calcium, folate, iodine, iron, magnesium, zinc, selenium, chromium, manganese, molybdenum, phosphorus, copper, antioxidant, or any combination thereof.

In various embodiments, the nanoparticle or composition comprises one or more nutritional or bioactive agent in a concentration range of about 0.1 wt % to about 100 wt %. In some embodiments, the nanoparticle or composition comprises one or more nutritional or bioactive agents in a concentration range of about 1 wt % to about 100 wt %. In some embodiments, the nanoparticle or composition comprises one or more nutritional or bioactive agents in a concentration range of about 1 wt % to about 70 wt %. In some embodiments, the nanoparticle or composition comprises one or more nutritional or bioactive agents in a concentration range of about 10 wt % to about 80 wt %. In some embodiments, the nanoparticle or composition comprises one or more nutritional or bioactive agents in a concentration range of about 10 wt % to about 70 wt %. In some embodiments, the nanoparticle or composition comprises one or more nutritional or bioactive agents in a concentration range of about 10 wt % to about 50 wt %. In some embodiments, the nanoparticle or composition comprises one or more nutritional or bioactive agents in a concentration range of about 15 wt % to about 45 wt %. In some embodiments, the nanoparticle or composition comprises one or more nutritional or bioactive agents in a concentration range of about 35 wt % to about 40 wt %.

For example, in some embodiments, the nanoparticle or composition comprises one or more nutritional or bioactive agents in a concentration of about 1 wt %. In some embodiments, the nanoparticle or composition comprises one or more nutritional or bioactive agents in a concentration of about 2 wt %. In some embodiments, the nanoparticle or composition comprises one or more nutritional or bioactive agents in a concentration of about 5 wt %. In some embodiments, the nanoparticle or composition comprises one or more nutritional or bioactive agents in a concentration of about 5.5 wt %. In some embodiments, the nanoparticle or composition comprises one or more nutritional or bioactive agents in a concentration of about 10 wt %. In some embodiments, the nanoparticle or composition comprises one or more nutritional or bioactive agents in a concentration of about 13 wt %. In some embodiments, the nanoparticle or composition comprises one or more nutritional or bioactive agents in a concentration of about 15 wt %. In some embodiments, the nanoparticle or composition comprises one or more nutritional or bioactive agents in a concentration of about 20 wt %. In some embodiments, the nanoparticle or composition comprises one or more nutritional or bioactive agents in a concentration of about 25 wt %. In some embodiments, the nanoparticle or composition comprises one or more nutritional or bioactive agents in a concentration of about 30 wt %. In some embodiments, the nanoparticle or composition comprises one or more nutritional or bioactive agents in a concentration of about 35 wt %. In some embodiments, the nanoparticle or composition comprises one or more nutritional or bioactive agents in a concentration of about 37 wt %. In some embodiments, the nanoparticle or composition comprises one or more nutritional or bioactive agents in a concentration of about 40 wt %. In some embodiments, the nanoparticle or composition comprises one or more nutritional or bioactive agents in a concentration of about 45 wt %. In some embodiments, the nanoparticle or composition comprises one or more nutritional or bioactive agents in a concentration of about 50 wt %. In some embodiments, the nanoparticle or composition comprises one or more nutritional or bioactive agents in a concentration of about 60 wt %. In some embodiments, the nanoparticle or composition comprises one or more nutritional or bioactive agents in a concentration of about 70 wt %. In some embodiments, the nanoparticle or composition comprises one or more nutritional or bioactive agents in a concentration of about 80 wt %. In some embodiments, the nanoparticle or composition comprises one or more nutritional or bioactive agents in a concentration of about 90 wt %. In some embodiments, the nanoparticle or composition comprises one or more nutritional or bioactive agents in a concentration of about 95 wt %. In some embodiments, the nanoparticle or composition comprises one or more nutritional or bioactive agents in a concentration of about 95.5 wt %. In some embodiments, the nanoparticle or composition comprises one or more nutritional or bioactive agents in a concentration of about 99 wt %. In some embodiments, the nanoparticle or composition comprises one or more nutritional or bioactive agents in a concentration of about 99.9 wt %. In some embodiments, the nanoparticle or composition comprises one or more nutritional or bioactive agents in a concentration of about 100 wt %.

In various embodiments, the nanoparticle or composition further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In various embodiments, the nanoparticle or composition comprises one or more helper compound in a concentration range of about 0 wt % to about 100 wt %. In some embodiments, the nanoparticle or composition comprises one or more helper compound in a concentration range of about 0.01 wt % to about 99.99 wt %. In some embodiments, the nanoparticle or composition comprises one or more helper compound in a concentration range of about 0.1 wt % to about 99.9 wt %. In some embodiments, the nanoparticle or composition comprises one or more helper compound in a concentration range of about 0.1 wt % to about 90 wt %. In some embodiments, the nanoparticle or composition comprises one or more helper compound in a concentration range of about 0.1 wt % to about 70 wt %. In some embodiments, the nanoparticle or composition comprises one or more helper compound in a concentration range of about 5 wt % to about 95 wt %. In some embodiments, the nanoparticle or composition comprises one or more helper compound in a concentration range of about 0.5 wt % to about 50 wt %. In some embodiments, the nanoparticle or composition comprises one or more helper compound in a concentration range of about 0.5 wt % to about 47 wt %. In some embodiments, the nanoparticle or composition comprises one or more helper compound in a concentration range of about 2.5 wt % to about 47 wt %.

For example, in some embodiments, the nanoparticle or composition comprises one or more helper compound in a concentration of about 0.01 wt %. In some embodiments, the nanoparticle or composition comprises one or more helper compound in a concentration of about 0.1 wt %. In some embodiments, the nanoparticle or composition comprises one or more helper compound in a concentration of about 0.5 wt %. In some embodiments, the nanoparticle or composition comprises one or more helper compound in a concentration of about 1 wt %. In some embodiments, the nanoparticle or composition comprises one or more helper compound in a concentration of about 1.5 wt %. In some embodiments, the nanoparticle or composition comprises one or more helper compound in a concentration of about 2 wt %. In some embodiments, the nanoparticle or composition comprises one or more helper compound in a concentration of about 2.5 wt %. In some embodiments, the nanoparticle or composition comprises one or more helper compound in a concentration of about 5 wt %. In some embodiments, the nanoparticle or composition comprises one or more helper compound in a concentration of about 10 wt %. In some embodiments, the nanoparticle or composition comprises one or more helper compound in a concentration of about 12 wt %. In some embodiments, the nanoparticle or composition comprises one or more helper compound in a concentration of about 15 wt %. In some embodiments, the nanoparticle or composition comprises one or more helper compound in a concentration of about 16 wt %. In some embodiments, the nanoparticle or composition comprises one or more helper compound in a concentration of about 20 wt %. In some embodiments, the nanoparticle or composition comprises one or more helper compound in a concentration of about 25 wt %. In some embodiments, the nanoparticle or composition comprises one or more helper compound in a concentration of about 30 wt %. In some embodiments, the nanoparticle or composition comprises one or more helper compound in a concentration of about 35 wt %. In some embodiments, the nanoparticle or composition comprises one or more helper compound in a concentration of about 37 wt %. In some embodiments, the nanoparticle or composition comprises one or more helper compound in a concentration of about 40 wt %. In some embodiments, the nanoparticle or composition comprises one or more helper compound in a concentration of about 45 wt %. In some embodiments, the nanoparticle or composition comprises one or more helper compound in a concentration of about 46.5 wt %. In some embodiments, the nanoparticle or composition comprises one or more helper compound in a concentration of about 47 wt %. In some embodiments, the nanoparticle or composition comprises one or more helper compound in a concentration of about 50 wt %. In some embodiments, the nanoparticle or composition comprises one or more helper compound in a concentration of about 60 wt %. In some embodiments, the nanoparticle or composition comprises one or more helper compound in a concentration of about 63 wt %. In some embodiments, the nanoparticle or composition comprises one or more helper compound in a concentration of about 70 wt %. In some embodiments, the nanoparticle or composition comprises one or more helper compound in a concentration of about 80 wt %. In some embodiments, the nanoparticle or composition comprises one or more helper compound in a concentration of about 90 wt %. In some embodiments, the nanoparticle or composition comprises one or more helper compound in a concentration of about 95 wt %. In some embodiments, the nanoparticle or composition comprises one or more helper compound in a concentration of about 95.5 wt %. In some embodiments, the nanoparticle or composition comprises one or more helper compound in a concentration of about 99 wt %. In some embodiments, the nanoparticle or composition comprises one or more helper compound in a concentration of about 100 wt %.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

In some embodiments, the cholesterol lipid is cholesterol or a derivative thereof, such as a substituted cholesterol molecule. In some embodiments, the nanoparticle or composition comprises a mixture of cholesterol and a substituted cholesterol molecule.

In some embodiments, the polymer is polyethylene glycol (PEG) or a derivative thereof.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl) cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In one embodiment, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

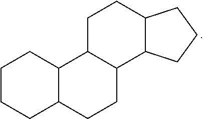 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the stabilizing lipid is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀) carbamyl]-1,2-dimyristyloxlpropyl-3-amine (PEG-c-DMA). In one embodiment, the polyethylene glycol-lipid is PEG-c-DOMG). In other embodiments, the nanoparticles comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy (polyethoxy)ethyl) butanedioate (PEG-S-DMG), a pegylated ceramide (PEG-cer), or a PEG dialkoxypropylcarbamate such as ω-methoxy (polyethoxy)ethyl-N-(2,3-di(tetradecanoxy)propyl) carbamate or 2,3-di(tetradecanoxy)propyl-N-(ω-methoxy (polyethoxy)ethyl) carbamate.

In one embodiment, the nanoparticle or composition is a polymersome. Any polymersome known in the art may be utilized. Thus, in one embodiment, the nanoparticle or composition comprises a homopolymer. In some embodiments, the nanoparticle or composition comprises a block copolymer that is a triblock, tetrablock, pentablock, or at least six block copolymer. In some embodiments, the nanoparticle or composition comprises poly(ethylene oxide) (PEO) block copolymer, poly(ethylethylene) (PEE), poly(butadiene) (PB or PBD), poly(styrene) (PS), poly(isoprene) (PI), PEI, poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-gluatamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, or any combination thereof.

In some embodiments, the nanoparticle or composition comprises poly(ε-caprolactone) (PCL) diblock co-polymer. In some embodiments, the nanoparticle or composition comprises poly(ethylene oxide)-block-poly(ε-caprolactone) (PEO-b-PCL) based diblock copolymers. In some embodiments, the nanoparticle or composition is derived from the coupling of poly(lactic acid), poly(glycolide), poly(lactic-coglycolic acid) and/or or poly(3-hydroxybutyrate) with PEO. In some embodiments, the nanoparticle or composition comprises PLGA. In some embodiments, the nanoparticle or composition comprises PEG. In one embodiment, the nanoparticle or composition comprises poly(lactide-co-glycolic acid)-polyethylene glycol (PLGA-PEG). For example, in some embodiments, a PLGA-PEG polymersome encapsulates the ICGJ and optionally PEI.

In one embodiment, the nanoparticle further comprises a cationic polymer. The cationic polymer may be a straight chain polymer (i.e., linear polymer) or a branched chain polymer (i.e., branched polymer), including hyperbranched polymers. In one embodiment the cationic polymer is a branched cationic polymer. In one embodiment, the cationic polymer is cross-linked. In one embodiment, the cationic polymer is a polyamine. In one embodiment, the cationic polymer has molecular weight of 5 kDa-3000 kDa. For example, in one embodiment, the cationic polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

In one embodiment, the cationic polymer is a polyalkyleneimine (e.g., polyethyleneimine), polyallylamine, polyamidoamine, or poly(amino-co-ester). In one embodiment, the cationic polymer is polyethyleneimine (PEI), chitosan, poly(2-N,N-dimethylaminoethylmethacrylate), or poly-L-lysine. In one embodiment, the cationic polymer stabilizes the nanocapsule.

In some embodiments, the nanoparticles comprise a biodegradable polymer comprises PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride), and a combination thereof.

In various embodiments, the nanoparticle has an average hydrodynamic diameter of from about 10 nm to about 10,000 nm, from about 130 nm to about 2,500 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 200 nm, 300 nm, 400 nm, 500 nm, 600 nm, 700 nm, 800 nm, 900 nm, 1,000 nm, 2,000 nm, 2,500 nm, 5,000 nm, or 10,000 nm.

For example, in various embodiments, the nanoparticle has an average particle size (e.g., average hydrodynamic diameter of the nanoparticle) below about 2,500 nm. In some embodiments, the nanoparticle has a particle size (e.g., average hydrodynamic diameter of the nanoparticle) of between about 1 nm to about 2,500 nm. In some embodiments, the nanoparticle has a particle size (e.g., average hydrodynamic diameter of the nanoparticle) of between about 1 nm to about 300 nm. In some embodiments, the nanoparticle has a particle size (e.g., average hydrodynamic diameter of the nanoparticle) of between about 1 nm to about 200 nm.

In various embodiments, the nanoparticle of the present invention is substantially non-toxic.

In one embodiment, the nanoparticle is a biodegradable nanoparticle. In one embodiment, the nanoparticle is a biodegradable edible nanoparticle.

In some embodiments, the nanoparticle is any type of nanoparticle, including, but not limited to, liposomes, lipid nanoparticles, organic nanoparticles, inorganic nanoparticles (e.g., metal nanoparticles, such as gold nanoparticles, iron nanoparticles, ZnO nanoparticles, TiO₂ nanoparticles, etc.), biocompatible nanoparticles, such as biocompatible organic nanoparticles, biocompatible inorganic nanoparticles, etc., polymer nanoparticles, nanoclusters, nanocapsules, core-shell nanocapsules, nanovesicles, micelles, block copolymer micelles, lamaellae shaped particles, polymersomes, dendrimers, emulsions, exosomes, SEDDS, microspheres, micro-structured lipid carriers, nano-structured lipid carriers, and other nano-size particles of various other small fabrications that are known to those of skill in the art.

For example, in some embodiments, the nanoparticle is a liposome, lipid nanoparticle, nanocapsule, nanocarrier, nanoemulsion, micelle, solid lipid nanoparticle (SLN), nanostructured lipid carrier (NLC), modified lipid nanoparticle, or any combination thereof.

In one embodiment, the nanoparticle is a liposome. In one embodiment, the nanoparticle is a biodegradable liposome. In one embodiment, the nanoparticle is a biodegradable edible liposome. In some embodiments, the liposome comprises phospholipids, cholesterol, sphingolipids, ceramides, hapten-conjugated lipids, or any combination thereof.

In one embodiment, the nanoparticle is a lipid nanoparticle. In some embodiments, the lipid nanoparticles comprise phospholipids, cholesterol, PEG-phospholipids, PEG-fatty acids, lipid-soluble vitamins, lipid conjugates, or any combination thereof.

In various embodiments, the nanoparticle comprises at least one agent (e.g., a nutritional or bioactive agent, nutraceutical agent, therapeutic agent, etc.). In some embodiments, the nanoparticle encapsulates at least one agent. Thus, in one embodiment, the nanoparticle is a nanocapsule. In one embodiment, the nanoparticle is biodegradable nanocapsule. In one embodiment, the nanoparticle is biodegradable edible nanocapsule. In one embodiment, the nanoparticle is a nanocarrier.

In some embodiments, the agent is any agent described herein, such as any nutritional or bioactive agent (e.g., vitamins, minerals, etc.), drug, lipid, polymer, peptide, polypeptide, and/or polypeptide variant described herein.

In some embodiments, the agent is adhered to the surface of the nanoparticle of the invention. In some embodiments, the agent is integrated into the structure of the nanoparticle of the invention. In some embodiments, the nanoparticle of the invention encapsulates at least one agent. In some embodiments, the nanoparticle of the invention comprises at least two agents.

In one aspect, the invention is not limited to any particular cargo or agent for which the nanoparticle of the invention is able to carry or transport. Rather, the invention includes any agent that can be carried by the nanoparticle of the invention. For example, the agents that can be carried by the nanoparticle of the invention include, but are not limited to, nutritional or bioactive agents and therapeutic agents.

In one aspect of the invention, the agent is a therapeutic agent. In one embodiment, the nanoparticle or composition of the invention encapsulates the therapeutic agent. In one embodiment, the nanoparticle or composition of the invention is bound to the therapeutic agent. In one embodiment, the therapeutic agent is a hydrophobic therapeutic agent. In one embodiment, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional or bioactive agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent is one or more non-therapeutic moieties. In some embodiments, the nanoparticle or composition of the invention comprises one or more therapeutic moieties, one or more non-therapeutic moieties, or any combination thereof.

In some embodiments, the nanoparticle or composition further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle or composition taking into consideration at least the type of therapy to be performed.

One of skill in the art would understand that different embodiments of nanoparticles disclosed herein can be used in connection with any aspect of the described invention.

### Nanoparticle Compositions

In one aspect, the present invention relates to a composition comprising at least one nanoparticle of the present invention. In some embodiments, the composition comprises an edible coating, packing material, food preparation element, or any combination thereof. Examples of such packing materials include, but are not limited to, a plastic wrap, paper wrap, aluminum wrap, butcher paper, parchment paper, wax paper, aluminum foil, plastic food container, paper food container, aluminum food container, ceramics food container, glass food container, metal food container, wood food container, or any combination thereof.

Examples of such food preparation elements include, but are not limited to, a knife, spatula, bowl, table, counter, spoon, fork, gloves, cutting board, conveyor belt, grinder, food processor, blender, cup, glass, pan, or any combination thereof.

In some embodiments, the composition comprises plastic wrap coated with at least one nanoparticle of the present invention. In some embodiments, the composition comprises paper wrap coated with at least one nanoparticle of the present invention. In some embodiments, the composition comprises aluminum wrap coated with at least one nanoparticle of the present invention. In some embodiments, the composition comprises butcher paper coated with at least one nanoparticle of the present invention. In some embodiments, the composition comprises wax paper coated with at least one nanoparticle of the present invention. In some embodiments, the composition comprises parchment paper coated with at least one nanoparticle of the present invention. In some embodiments, the composition comprises aluminum foil coated with at least one nanoparticle of the present invention. In some embodiments, the composition comprises plastic container coated with at least one nanoparticle of the present invention. In some embodiments, the composition comprises paper container coated with at least one nanoparticle of the present invention. In some embodiments, the composition comprises aluminum container coated with at least one nanoparticle of the present invention. In some embodiments, the composition comprises ceramics food container coated with at least one nanoparticle of the present invention. In some embodiments, the composition comprises glass food container coated with at least one nanoparticle of the present invention. In some embodiments, the composition comprises metal food container coated with at least one nanoparticle of the present invention. In some embodiments, the composition comprises wood food container coated with at least one nanoparticle of the present invention.

In some embodiments, the composition comprises a knife coated with at least one nanoparticle of the present invention. In some embodiments, the composition comprises a spatula coated with at least one nanoparticle of the present invention. In some embodiments, the composition comprises a bowl coated with at least one nanoparticle of the present invention. In some embodiments, the composition comprises a table coated with at least one nanoparticle of the present invention. In some embodiments, the composition comprises a counter coated with at least one nanoparticle of the present invention. In some embodiments, the composition comprises a spoon coated with at least one nanoparticle of the present invention. In some embodiments, the composition comprises a fork coated with at least one nanoparticle of the present invention. In some embodiments, the composition comprises gloves coated with at least one nanoparticle of the present invention. In some embodiments, the composition comprises a cutting board coated with at least one nanoparticle of the present invention. In some embodiments, the composition comprises a conveyor belt coated with at least one nanoparticle of the present invention. In some embodiments, the composition comprises a grinder coated with at least one nanoparticle of the present invention. In some embodiments, the composition comprises a food processor coated with at least one nanoparticle of the present invention. In some embodiments, the composition comprises a blender coated with at least one nanoparticle of the present invention. In some embodiments, the composition comprises a cup coated with at least one nanoparticle of the present invention. In some embodiments, the composition comprises a glass coated with at least one nanoparticle of the present invention. In some embodiments, the composition comprises a pan coated with at least one nanoparticle of the present invention.

In some embodiments, the composition comprises at least two different nanoparticles comprising different agents. In some embodiments, the composition comprises multiple nanoparticles. For example, in one embodiment, the composition comprises at least two different nanoparticles comprising different agents where the first nanoparticle comprises at least one vitamin and the second nanoparticle comprises at least one mineral or at least one different vitamin.

Thus, in some embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

In one embodiment, the nanoparticle of the invention comprises one or more linker molecules. Examples of linker molecules include, but are not limited to, azide functionalized molecules, maleimide functionalized molecules, carboxyl functionalized molecules, amine functionalized molecules, hydrazine functionalized molecules, dibenzo-cyclooctyne functionalized molecules, or any combination thereof.

In one embodiment, the nanoparticle of the invention comprises one or more functional groups. In some embodiments, the functional group is an azide functional group, maleimide functional group, carboxyl functional group, amine functional group, hydrazine functional group, dibenzo-cyclooctyne functional group, or any combination thereof.

In one embodiment, the composition preserves at least one nutrient. For example, in some embodiments, the at least one nutrient is selected from vitamins (e.g., vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12), minerals (e.g., potassium, magnesium, calcium), antioxidant, or any combination thereof.

### Methods

In one aspect, the present invention provides a method of coating a surface of an element.

In one aspect, the present invention provides a method of reducing or inhibiting the activity or level of at least one microorganism on a surface of an element.

In one aspect, the present invention provides a method of preventing or reducing the growth of at least one microorganism on a surface of an element.

Thus, in one aspect, the present invention relates, in part, a method of reducing the perishability of food.

In another aspect, the present invention provides a method of increasing a shelf-life of food.

In another aspect, the present invention provides a method of preserving nutrients in food.

In another aspect, the present invention provides a method of maintaining nutrients in food.

In another aspect, the present invention provides a method of increasing or improving nutritional value in food.

In various embodiments, the method comprises administering an effective amount of at least one nanoparticle of the present invention to the surface of the element. In other embodiments, the method comprises administering an effective amount of at least one composition of the present invention to the surface of the element.

In some embodiments, the element is food, packing material, food preparation element, and any combination thereof. In one embodiment, food is a perishable food. Examples of such foods include, but are not limited to, meats, including poultry and seafoods, vegetables, fruits, herbs, dairies (e.g., cheeses, ice cream, etc.), eggs, egg products, nuts, seeds, soy, candies, grains, flour products (e.g., breads), gluten-free products, drinks and beverages (e.g., coffee, tea, nonalcoholic beverages, alcoholic beverages, etc.), deli, desserts, sweets, meal replacements, meal supplements, soups, broths, bouillons, condiments, sauces, whey, artificial sweeteners, salts, etc.

In some embodiments, the packing material is a plastic wrap, paper wrap, aluminum wrap, butcher paper, parchment paper, wax paper, aluminum foil, plastic food container, paper food container, aluminum food container, ceramics food container, glass food container, metal food container, wood food container, and any combination thereof.

In some embodiments, the food preparation element is a knife, spatula, bowl, table, counter, spoon, fork, gloves, cutting board, conveyor belt, grinder, food processor, blender, cup, glass, pan, and any combination thereof.

In some embodiments, the microorganism is any microorganism described herein, such as a bacterium (e.g., Escherichia, Escherichia coli, Salmonella enterica, Staphylococcus, Staphylococcus aureus, Listeria monocytogenes, Clostridium perfringens, Clostridium botulinum, Shigella boydii, Vibrio, Vibrio parahaemolyticus, Campylobacter, Campylobacter jejuni, Yersinia, Yersinia enterocolitica, Cronobacter sakazakii, Enterobacteriaceae, Erwinia herbicola, Rahnella aquatilis, Lacticaseibacillus casei, Leuconostoc mesenteroides, Bacillus cereus, Pseudomonadaceae, P. fluorescens, or any combination thereof), virus, pathogen, parasite, fungus, yeast (e.g., Candida sp., Candida pulcherrima, Candida humilis, Candida milleri, Candida tropicalis, Candida fermentati, Candida krusei, Candida parapsilosis, Candida guilliermondii, Candida lusitaniae, Candida rugosa, Cryptococcus sp., Rhodotorula sp., Trichosporon sp., Pichia sp., Torulaspora sp., C. lambica, C. sake, Debaryomyces polymorphus, or any combination thereof), mold (e.g., Botrytis, Botrytis cinerea, Rhizopus, Rhizopus stolonifer, Mucor, Mucor piriformis, Rhizoctonia solani, Phytophtora cactorum, Alternaria, Penicillium, Cladosporium, Aspergillus, Fusarium, Geotrichum, or any combination thereof), and any combination thereof.

In one aspect, the present invention relates, in part, a method of delivering an agent (e.g. nutritional or bioactive agent, nutraceutical agent, therapeutic agent, etc.) to a subject. In some embodiments, the method comprises administering an effective amount of at least one nanoparticle of the present invention to the subject. In some embodiments, the method comprises administering an effective amount of at least one composition of the present invention to the subject. For example, in some embodiments, the present invention provides a method of delivering a nutritional or bioactive agent to a subject. Examples of such nutritional or bioactive agent include, but are not limited to any nutritional or bioactive agents described herein, such as vitamins (e.g., vitamin A, vitamin B, such as vitamin B1, vitamin B2, vitamin B6, vitamin B12, etc.), vitamin C, vitamin D, vitamin E, and vitamin K), minerals (e.g., calcium, folate, iodine, iron, magnesium, zinc, phosphorus, selenium, chromium, manganese, molybdenum, and copper), niacin, pantothenic acid, biotin, coline, and any combination thereof.

In one aspect, the present invention provides a method of treating or preventing nutrient deficiency in a subject in need thereof.

In another aspect, the present invention relates, in part, a method of treating or preventing a disease, disorder, or condition.

In some embodiments, the method comprises administering an effective amount of at least one nanoparticle of the present invention or a composition thereof to the subject.

In some embodiments, the disease, disorder, or condition is selected from obesity, diabetes, cancer, neurodegenerative disease, neurodegenerative disorder, disease associated with nutrient deficiency, disorder associated with nutrient deficiency, condition associated with nutrient deficiency, or any combination thereof.

In some embodiments, the disease, disorder, or condition associated with nutrient deficiency is a disease, disorder, or condition associated with vitamin A deficiency, disease, disorder, or condition associated with vitamin B deficiency, (e.g., a disease, disorder, or condition associated with vitamin B1 deficiency, a disease, disorder, or condition associated with vitamin B2 deficiency, a disease, disorder, or condition associated with vitamin B6 deficiency, a disease, disorder, or condition associated with vitamin B12 deficiency, etc.), disease, disorder, or condition associated with vitamin C deficiency, disease, disorder, or condition associated with vitamin D deficiency, disease, disorder, or condition associated with vitamin E deficiency, disease, disorder, or condition associated with vitamin K deficiency, disease, disorder, or condition associated with calcium deficiency, disease, disorder, or condition associated with folate deficiency, disease, disorder, or condition associated with iodine deficiency, disease, disorder, or condition associated with iron deficiency, disease, disorder, or condition associated with magnesium deficiency, disease, disorder, or condition associated with zinc deficiency, disease, disorder, or condition associated with phosphorus deficiency, disease, disorder, or condition associated with copper deficiency, disease, disorder, or condition associated with niacin, disease, disorder, or condition associated with pantothenic acid deficiency, disease, disorder, or condition associated with biotin deficiency, disease, disorder, or condition associated with coline deficiency, disease, disorder, or condition associated with selenium deficiency, disease, disorder, or condition associated with chromium deficiency, disease, disorder, or condition associated with manganese deficiency, disease, disorder, or condition associated with molybdenum deficiency, or any combination thereof.

In some embodiments, the disease, disorder, or condition associated with nutrient deficiency is a night blindness, beri-beri, retarded growth, bad skin, anemia, scurvy, rickets, excessive bleeding after injury, brittle bones, bad teeth, bad bones, goiter, enlarged thyroid gland, low appetite, birth defects, immune disorders, chronic diseases, metabolic diseases, and any combination thereof.

In one embodiment, the method comprises administration of the composition to a subject. In certain embodiments, the method comprises administering a plurality of doses to the subject. In another embodiment, the method comprises administering a single dose of the composition, where the single dose is effective in delivery of the target therapeutic agent.

In one aspect, the composition of the present invention comprises one or more nanoparticle formulated for targeted delivery of an agent to a cell of interest. Examples of such agents include, but are not limited to, a nutritional or bioactive agent, nutraceutical agent, therapeutic agent, small molecule, peptide, polypeptide, amino acid molecule, nucleic acid molecule, drug, pro-drug, diagnostic agent, or any combination thereof.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition. In one embodiment, the method comprises transdermal delivery.

In some embodiments, administration comprises intravenous, intranasal, intramuscular, subcutaneous, or transdermal delivery of the nanoparticles or compositions of the present invention.

It will be appreciated that the composition of the invention may be administered to a subject either alone, or in conjunction with another agent.

The pharmaceutical compositions useful for practicing the invention may be administered to deliver a dose of from ng/kg/day and 100 mg/kg/day. In one embodiment, the invention envisions administration of a dose which results in a concentration of the compound of the present invention from 10 nM and 10 μM in a mammal.

Typically, dosages which may be administered in a method of the invention to a mammal, for example a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage of the compound will vary from about 0.1 μg to about 100 mg per kilogram of body weight of the mammal. In one embodiment, the dosage of the compound will vary from about 0.1 μg to about 50 mg per kilogram of body weight of the mammal. In one embodiment, the dosage of the compound will vary from about 0.1 μg to about 10 mg per kilogram of body weight of the mammal. In one embodiment, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a composition of the present invention may be performed by single administration or boosted by multiple administrations.

In one embodiment, the invention includes a method comprising administering a combination of compositions described herein. In certain embodiments, the combination has an additive effect, wherein the overall effect of the administering the combination is approximately equal to the sum of the effects of administering each composition. In other embodiments, the combination has a synergistic effect, wherein the overall effect of administering the combination is greater than the sum of the effects of administering each composition.

In one aspect, the therapeutic compounds or compositions of the invention may be administered prophylactically (i.e., to prevent nutrient deficiency, obesity, diabetes, cancer, neurodegenerative disease, neurodegenerative disorder, disease associated with nutrient deficiency, disorder associated with nutrient deficiency, condition associated with nutrient deficiency, etc.) or therapeutically (i.e., to treat nutrient deficiency, obesity, diabetes, cancer, neurodegenerative disease, neurodegenerative disorder, disease associated with nutrient deficiency, disorder associated with nutrient deficiency, condition associated with nutrient deficiency, etc.) to subjects suffering from or at risk of (or susceptible to) developing the nutrient deficiency, obesity, diabetes, cancer, neurodegenerative disease, neurodegenerative disorder, disease associated with nutrient deficiency, disorder associated with nutrient deficiency, condition associated with nutrient deficiency, etc. Such subjects may be identified using standard clinical methods.

In the context of the present invention, prophylactic administration occurs prior to the manifestation of overt clinical symptoms of disease, disorder, or condition, such that the disease, disorder, or condition is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the invention can be useful in combination with therapeutic, anti-cancer, and/or radiotherapeutic agents. Thus, the present disclosure provides a combination of the present nanoparticle with therapeutic, anti-cancer, and/or radiotherapeutic agents for simultaneous, separate, or sequential administration. The composition of the invention and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In one embodiment, such powders comprise particles wherein at least 98% of the particles by weight have a diameter greater than 0.5 nanometers and at least 95% of the particles by number have a diameter less than 7 nanometers. In one embodiment, at least 95% of the particles by weight have a diameter greater than 1 nanometer and at least 90% of the particles by number have a diameter less than 6 nanometers. In one embodiment, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some aspects, having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the certain embodiments of the present invention, and are not to be construed as limiting in any way the remainder of the disclosure.

### Example 1: Nanoencapsulated Natural Phytochemicals for an Edible Coating on Fresh-Cut Fruits and Vegetables (FCFVs)

The present example describes the investigation of nanoencapsulated natural phytochemicals for an edible coating on fresh-cut fruits and vegetables (FCFVs) to actively protect FCFVs as part of the package function and to effectively delivery and enhance body absorption of otherwise easily degraded nutrients with much-added nutraceutical values, thus balancing the cost-value of actively packaged FCFVs as effective nutraceutical foods for largescale market acceptance. For example, FCFVs can function as an active packaging for FCFV preservation, nutrition maintenance (function 1) and as novel nutraceutical foods with improved bioavailability (function 2).

Many natural phytochemicals from plants exhibit antioxidant, antimicrobial, antigenotoxic, anti-inflammatory, and antidiabetic functions, and therefore are attractive healthy supplements. They, however, are easily degraded during storage and easily metabolized in the gastrointestinal tract before entering the blood circulation. Nanoencapsulation is a potential route to deliver these phytochemicals with improved bioavailability and bioactivity.

Additionally, a good design of nutrition supplements should leverage a popular food as their carrier. FCFVs are such carriers. With their own rich nutrients and their convenient availability to consumers, FCFVs are a popular healthy food. However, with cuticle removal and cell damage, they are highly perishable. A short shelf life, waste generation, and hence high cost, as well as foodborne illness are associated problems that must be solved. Edible coating based active packaging, which aims to provide both a physical barrier against dehydration and gas permeation and chemical protection against microbes and other deleterious processes, has gained traction, particularly if using natural phytochemicals as antioxidants and antimicrobials. Nevertheless, the free form of these compounds in the coating is also easily degraded and to compensate for the degradation over time, large quantities need to be applied, which is not only very costly but can also negatively affect the sensory properties of foods. Consumer's perception and the high cost are barriers to the largescale acceptance of active packaging.

Based on the previous nanoencapsulated phytochemicals delivery for chronicle diseases (Zhang J, J Nutr Biochem. 2016; 30:14-23; Zhang J, J Agric Food Chem. 2013; 61:9200-9; Zu Y, Colloids Surf B Biointerfaces. 2018; 164:414-423; Zu Y, J Control Release. 2021; 333:339-351), the present studies utilized a holistic approach by designing a nanoencapsulated phytochemicals-based multifunctional coating on FCFVs as nutraceutical foods to solve the above-discussed problems. The present studies provided the development of nano-encapsulated phytochemicals-based nutraceutical coatings by: Task I: Synthesis and properties study of nanoencapsulations and coating films; Task II: Multifunctional nutraceutical coatings for FCFV preservation; and Task III: Bioavailability, bioactivities, and safety of nutraceutical coatings in research mice.

### FCFVs and Their Preservation

FCFVs are a healthy diet, but they are also known to harbor large bacterial populations (Leff J W, PloS one. 2013; 8: e59310), including those fatal human pathogens, such as E. coli, L. monocytogenes, and Salmonella spp. Since fresh produce is consumed raw, such pathogens can cause disease outbreaks. Indeed, FCFVs have emerged as a new vehicle of foodborne disease transmission. Microbes also critically impact the rates of food spoilage (Gram L, International journal of food microbiology. 2002; 78:79-97), leading to unfavorable organoleptic profile, short shelf life, and huge amount of wastes and economic losses. With cuticle removal and cell damage, FCFVs are more prone to contamination and subsequent deterioration and spoilage.

FCFVs are evaluated based on their sensorial quality like appearance, color, flavor, texture, nutritional quality as concentrations of vitamins, phytochemicals, minerals, and safety aspects. During storage, many different biochemical processes occur (Finnegan E, Postharvest Biology and Technology. 2015; 100:91-98). In addition to microbial contamination, reaction of polyphenol oxidase with the phenols causes browning; pectin catalyzed by pectin methylesterase and polygalacturonase leads to softening; and transformation of phytochemicals results in nutritional loss, especially for various vitamins. These changes as well as loss of moisture, chlorophyll degradation, and fermentation are easily noticed in the daily life, such as in various fresh produces (e.g., apple, strawberry, lettuce, and cucumbers, etc.).

Of different strategies to maintain safety and extend shelf life, chemical treatment, such as calcium dip and modified atmosphere packaging, have been widely applied in FCFV production. Various radiation-based antioxidation and/or disinfection technologies, such as UV light, cold plasma, and gramma ray, have been under investigation. Since FCFVs are essentially wounded tissues, using an artificial edible skin would significantly improve the protection of FCFVs (function 1). Indeed, edible coatings have long been applied to extend shelf life of uncut fruits, which are formed by dipping or spraying with a range of edible materials. This semipermeable artificial skin retards dehydration, regulate respiration and ripening, restricts volatile flavors loss, and provides antimicrobial and many other functions.

In strive to address the problem of plastic pollution, the concept of edible packaging is becoming popular. However, just like ice cream cones are wrapped in paper and sold in a box, edible coatings of FCFVs are not to replace a physical wrapping layer and a packaging box. Furthermore, the edible coatings on FCFVs must have good taste and texture for consumer acceptance. A mechanically strong and gas/vapor-tight edible layer on FCFVs is not desirable. Therefore, the ideal edible layer coated FCFVs still have additional physical package.

### Edible Coating Materials Selection

To be an edible and protective layer, the coating materials must be from food constituents, can easily form very thin coating without cracks, and have the properties to fulfill the semipermeable skin functions. Lipids (e.g., fatty acids, waxes, neutral lipids, and resins), polysaccharides (e.g., cellulose, chitosan, alginates, and starch) and proteins (e.g., zein, soy protein, whey, and casein), alone or in combination, are common edible coating candidates (Hassan B, International journal of biological macromolecules. 2018; 109:1095-1107; Mohamed S A, Carbohydrate polymers. 2020; 238:116178). The traditional lipid-based edible coatings exhibit much better vapor and gas barrier properties and surface appearance than polysaccharides- and protein-based coatings. But a greasy surface and unpleasant organoleptic characteristics are undesirable, particularly for FCFVs (Min S, Improving the safety of fresh fruit and vegetables: Elsevier; 2005:454-492). The nonpolymeric nature of lipids also limits their cohesive coating-forming capacity. Polysaccharides (Cazón P, Food Hydrocolloids. 2017; 68:136-148) and proteins are polymeric and hydrophilic, thus generally good coating formers with excellent isolation performance for gases, aroma and lipids but not for moisture. Due to strong cohesive energy of polymers, their coatings are also susceptible to crack. Compatible plasticizers can be added to improve the viscoelasticity of the coating solution and prevent cracks.

With the pros and cons of these coating materials, recent interest has been on composite coatings by using hydrophilic polysaccharides or proteins as the coating matrix wherein lipids are dispersed and entrapped. Such a composite emulsion coating are an excellent barrier for both gases and moisture.

Since the present studies focus on nanoencapsulation of phytochemicals but not coating-forming materials, chitosan (CS) (van den Broek L A, Carbohydrate polymers. 2015; 116:237-242; Elsabee M Z, Materials Science and Engineering: C. 2013; 33:1819-1841; Gol N B, Postharvest Biology and Technology. 2013; 85:185-195; Kumar S, Trends in Food Science & Technology. 2020; 97:196-209), a widely demonstrated edible biopolymer as the main coating component. Others, such as starch, can also be used to replace CS. CS can easily form trans-parent coatings and was compatible with other substances used in the coating. Strikingly, CS itself has broad antimicrobial activities by inhibiting spore germination and pathogen growth (Verlee A, Carbohydrate polymers. 2017; 164:268-283). Its attractive antifungal property comes from its ability to induce chitinase, which can destroy fungal cell walls (Hirano S, Agricultural and biological chemistry. 1989; 53:3065-3066). To improve basic coating functionality, plasticizers (e.g., glycerol) for enhanced mechanical properties and emulsifiers/surfactants for stabilization of emulsion are often required.

The coating also optionally contains antimicrobial agents (e.g., sorbic acid and essential oils) and antioxidants (e.g., citric acid and α-tocopheryl acetate) as well as nutrients (e.g., calcium, zinc, or vitamin E), flavor, and coloring agents. The present example focused on four natural phytochemicals, namely trans-resveratrol, quercetin, curcumin, and epigallocatechin gallate (EGCG), as antimicrobial and antioxidant agents and as value-added nutraceutical components (function 2).

### Coated FCFVs as Value-Added Nutraceuticals

With the protection of nanoencapsulation-based edible coatings, the nutritional qualities of FCFVs are preserved. Their consumption is conducive for improving health status and preventing chronic diseases including obesity, diabetes, and other metabolic diseases. However, previous studies have clearly demonstrated that many very useful phytochemicals, even though well preserved before consumption, can hardly enter blood to be absorbed for functioning. They generally have low aqueous solubility and are easily degraded in the digest tractions, resulting in very low bioavailability.

Trans-resveratrol (3,5,4′-tri-hydroxy-trans-stilbene, RES or R) is a natural polyphenolic compound found in various foods such as grapes, red wines, and mulberries (Feijóo O, Journal of Food Composition and Analysis. 2008; 21:608-613; Dixon R A, The plant cell. 1995; 7:1085; Burns J, J Agric Food Chem. 2002; 50:3337-40; Hurst W J, J Agric Food Chem. 2008; 56:8374-8). Many cell and animal studies have re-ported that RES has beneficial effects on longevity (Finkel T, Nature. 2003; 425:132), anti-inflammation (Harikumar K B, Cell cycle. 2008; 7:1020-1035; Poulsen M M, Molecular Basis of Disease. 2015; 1852:1124-1136), anti-obesity (Kwon J Y, Nutrition research. 2012; 32:607-616; Szkudelska K, European journal of pharmacology. 2010; 635:1-8), anti-cancer (Jang M, Science. 1997; 275:218-220; Gwak H, Cancer letters. 2016; 371:347-353), anti-diabetes (Chen S, Digestive and Liver Disease. 2015; 47:226-232) and improvement of cardiovascular and metabolic health (Huang H, Obesity Reviews. 2016; 17:1329-1340; Goldberg D M, Clinical biochemistry. 2003; 36:79-87). However, the evidence is inconclusive in human studies regarding its effectiveness on improving metabolic health (Barger J L, Acad Sci. 2013; 1290:122-129; Poulsen M M, Acad Sci. 2013; 1290:74-82), most likely due to its low aqueous solubility (<0.1 mg/mL; Bonechi C, PLOS One. 2012; 7: e41438.), trivial bioavailability (peak plasma RES concentration <10 μM after high dose oral administration (Goldberg D M, Clin Biochem. 2003; 36:79-87; Boocock D J, Cancer Epidemiol Biomarkers Prev. 2007; 16:1246-1252) due to degradation (Brill S S, J Pharm Pharmacol. 2006; 58:469-479)) and non-targeting specificity. RES also has antimicrobial functions by altering the expression of virulence factors (Vestergaard M, Int J Antimicrob Agents. 2019; 53:716-723) and inhibiting growth of a wide variety of bacteria and fungi, including Gram-positive bacteria (Ferreira S, J Sci Food Agric. 2016; 96:4531-4535), Gram-negative bacteria (Sun D, ChemMedChem. 2012; 7:1541-1545), and fungi (Chan M M., Biochem Pharmacol. 2002; 63:99-104). The working mechanisms include inhibition of ATP synthase and oxidative phosphorylation, induction of DNA fragmentation and concomitant upregulation of the SOS stress-response and membrane damage, decrease in cell division (Vestergaard M, Int J Antimicrob Agents. 2019; 53:716-723).

Quercetin (3,3′,4′,5′-7-pentahydroxy flavone, Q) is a flavonoid, abundant in apples, berries and onions. The antioxidant activities of Q are associated with the number and position of the free hydroxyl groups in its molecule (Kumari A, Colloid Surface B. 2010; 80:184-192). It has anticancer, anti-inflammation, antioxidant, and antiviral activities as well (Sun M, Colloids Surf B Biointerfaces. 2014; 113:15-24). However, it is chemically unstable, especially in aqueous alkaline medium (Zheng Y, J Pharm Sci. 2005; 94:1079-1089). The use of quercetin in pharmaceutical field is limited due to its low aqueous solubility and unstable property in physiological medium (Kumari A, PLOS One. 2012; 7: e41230). The solubility of quercetin in water is less than 0.003 g/L at 25° C. (Sun M, Colloids Surf B Biointerfaces. 2014; 113:15-24). It has a low level of bioavailability, and a high level of first pass metabolism in vivo before reaching the blood (Kumari A, PLOS One. 2012; 7: e41230). Quercetin has a broad-spectrum antimicrobial property by inhibiting growth of pathogenic bacteria such as Pseudomonas aeruginosa, Salmonella enteritidis, and Escherichia coli (Yang D, Oxid Med Cell Longev. 2020; 2020:8825387; Wang S, J Food Prot. 2018; 81:68-78). The major antibacterial mechanisms are to destroy the bacterial cell wall, to changing the cell permeability, to inhibit nucleic acid synthesis, to decrease protein expression and synthesis, and to reduce enzyme activities.

Curcumin (C) is the principal curcuminoid of the spice turmeric, the ground rhizome of the herb Curcuma longa. It has antioxidant, anti-inflammatory, anti-obesity, anti-angiogenesis, and anticarcinogenic properties. However, it has a low level of aqueous solubility, chemical stability, poor pharmacokinetic/pharmacodynamic (PK/PD), limited tissue distribution, and extensive metabolism (Nelson K M, J Med Chem. 2017; 60:1620-1637). Curcumin solubility in water is less than 0.1 mg/mL. At pH>7, the curcumin molecules are extremely unstable, with a half-life of <10 minutes (Tonnesen H H, Z Lebensm Unters Forsch. 1985; 180:402-404). Negligible amounts of curcumin were found in the blood of rats after oral administration of 1 g/kg of curcumin, indicating poor absorption from the gut (Wahlstrom B, Acta Pharmacol Toxicol (Copenh). 1978; 43:86-92). When curcumin was given orally at a dose of 2 g/kg to humans, extremely low curcumin concentrations (0.006 μg/mL) were detected in the blood (Shoba G, Planta Med. 1998; 64:353-356). Curcumin is an antimicrobial compound (Adamczak A, Pharmaceuticals (Basel). 2020; 13). It inhibits growth of Gram-positive bacteria (e.g., Staphylococcus aureus, S. epidermidis), spore-forming bacilli (e.g., Bacillus and Clostridium species), Gram-negative bacteria (e.g., Escherichia coli, Proteus mirabilis), as well as fungal (e.g., Candida stellatoidea, Scopulariopsis brevicaulis (Adamczak A, Pharmaceuticals (Basel). 2020; 13)).

Epigallocatechin Gallate (EGCG) is very abundant in green tea, comprising 48-55% of total green tea catechins (Wang S, J Nutr Biochem. 2006; 17:492-498; Basu A, Nutr Rev. 2007; 65:361-75). One 2 g green tea bag contains about 330 mg of EGCG. EGCG exhibits antioxidant, anti-inflammation, anti-obesity, antiatherosclerosis, anti-HIV and anticarcinogenic properties. Studies indicate that EGCG could maintain metabolic health, but the evidence is inconclusive regarding its effectiveness (Wolfram S, J Am Coll Nutr. 2007; 26: 373S-388S; Arab L, Stroke. 2009; 40:1786-1792). The major problems are its low stability and extensive metabolism in humans or research animals (Chen L, Drug Metab Dispos. 1997; 25:1045-1050; Warden B A, J Nutr. 2001; 131:1731-1737; Lee M J, Cancer Epidemiol Biomarkers Prev. 2002; 11:1025-1032). The blood peak concentrations of green tea catechins appear at 2 to 4 hours after oral administration. The absolute oral bioavailability of EGCG after drinking tea containing catechins at 10 mg/kg body weight is about 0.1% in humans and research animals (Warden B A, J Nutr. 2001; 131:1731-1737; Lambert J D, J Nutr. 2003; 133: 3262S-3267S). The peak plasma EGCG concentration is 0.15 UM after drinking 2 cups of green tea (Warden B A, J Nutr. 2001; 131:1731-1737; Lee M J, Cancer Epidemiol Biomarkers Prev. 2002; 11:1025-1032). Moreover, EGCG is unstable in both water and physiological fluid in vitro (Barras A, Int J Pharm. 2009; 379:270-277; Lambert J D, J Nutr. 2003; 133:4172-4177). EGCG stability is decreased by various metabolic transformations including methylation, glucuronidation, sulfation and oxidative degradation in vivo (Dou Q P, Nutr Cancer. 2009; 61:827-835; Lu H, Drug Metab Dispos. 2003; 31:452-461; Lu H, Drug Metab Dispos. 2003; 31:572-579; Vaidyanathan J B, Drug Metab Dispos. 2002; 30:897-903). EGCG has a remarkable antimicrobial activity (Gopal J, Sci Rep. 2016; 6:19710). It can inhibit growth Staphylococcus aureus, Staphylococcus epidermidis, Staphylococcus haemolyticus, E. coli, Salmonella typhi, and Proteus mirabilis. After EGCG binds to bacterial membrane, it can generate hydrogen peroxide to damage the membrane (Jeon J, Ann Dermatol. 2014; 26:564-569).

From the highlights on the four phytochemical models, it was concluded that phytochemicals are very useful bio-compounds and have a positively impacting on human health. Phytochemicals are also excellent edible antioxidants and antimicrobials for edible package coatings, and have very low solubility, stability, and bioavailability.

Hence, there are critical needs to encapsulate them into biocompatible and biodegradable nanoparticles (NPs) to increase their stability, bioavailability, and bioactivities for both functions of edible packaging and functional nutraceuticals.

### Nanoencapsulation for Protective Packaging and for Nutraceutical Delivery

Nanoencapsulation is the process to encapsulate free active agents into nanoscale carriers. Such formed nanoscale structures or NPs attain unique properties. Encapsulations protected active agents from degradation against ambient stresses. This was true for antioxidants and antimicrobials in the edible coatings. It was also true in protecting nutrients from degradation and generation of potentially harmful waste when passing through the digest tract. Thus, the quantities needed as protection agents were dramatically reduced, beneficial for both the taste of FCFVs and the cost. Furthermore, nanoencapsulation provided a much high surface-area/volume ratio compared to large particles, therefore they had better functional properties, such as high solubility, high adsorption, controlled-release, and increased bioavailability. NPs did not generally affect the organoleptic properties of food as opposed to the micron-sized particles. With various bacteria and fungi as potent microbe contaminants, combination of different agents to achieve synergistic antimicrobial activities was highly useful (da Silva C R, Antimicrob Agents Chemother. 2014; 58:1468-1478), and nanoencapsulation was able to blend several active agents together in same NPs. For phytochemicals delivery, NPs were extravasated effectively into tissues (Zhang L, Clin Pharmacol Ther. 2008; 83:761-769; Peer D, Nat Nanotechnol. 2007; 2:751-760) and cleared much slower than large carriers by the reticule-endothelial system in the liver and spleen (Nishiyama N, Nat Nanotechnol. 2007; 2:203-204). Protection from degradation and controlled release of antimicrobials/antioxidants over time was effective for maintaining FCFVs quality and extending their shelf life. Delivery of phytochemicals without degradation and simultaneously enhancing blood absorption significantly improved their bioavailability. Therefore, nanoencapsulation of phytochemicals as antioxidants/antimicrobials/nutrients were crucial to achieve the goal of designing effective FCFVs-based nutraceuticals with extended shelf life.

### Selection of Bio-Compatible NP Structures

The major biocompatible and biodegradable NPs were liposomes, nanoemulsions, micelles, solid lipid nanoparticles (SLNs), and nanostructured lipid carriers (NLCs) (Wang S, J Nutr Biochem. 2014; 25:363-376). The latter one has the most advanced bio-NP structure with a hydrophilic shell and a hydrophobic lipid core. The major advantages of NLCs over other NPs were the lack of organic solvents during the synthesis process, the long-time physical stability, the increase of loading capacity and encapsulation efficiency, and the possibility of protection of chemically labile compounds inside the NLC (Wang S, J Nutr Biochem. 2014; 25:363-376). Hence, NLCs were used to encapsulated NPs in the present studies.

The lipid core of the NLCs was modified by replacing conventionally used mono-, di-, or triglycerides, fatty acids, and waxes with vitamin E (α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, δ-tocotrienol, 8-tocotrienol acetate, δ-tocotrienol nicotinate). It was well known that mono-, di-, or tri-glycerides and fatty acids are high energy molecules and that increased consumption of these lipids were positively correlated with high risks of obesity, cardiovascular disease, and diabetes. Previous studies have successfully replaced these lipids with α-tocopherol acetate (Sigma T3001, melting temperature 25° C.) and (±)-α-tocopherol nicotinate (Sigma T 5134, melting temperature 38° C.). These two vitamin E derivatives have different structures, which led to high loading capacity. Additionally, α-tocopherol acetate and (±)-α-tocopherol nicotinate had many beneficial effects, including reduction of antioxidative stress, promotion of immune functions, and inhibition of cancer cell growth. Furthermore, α-tocopherol acetate and α-tocopherol nicotinate did not only protect the encapsulated phytochemicals, but also the coated fruits and vegetables. To further enhance the stability and activity, NLCs were coated with CS (CSNLCs). All materials used were generally recognized as safe (GRAS) by FDA. Phytochemicals, including RES, EGCG, quercetin, and curcumin, are encapsulated into these NPs.

### Multifunctional Edible Coatings on FCFVs as Nutraceutical Foods

Edible coatings are formed on FCFVs via dip-coating in the solution of the phytochemical-encapsulated bio-NPs and CS biopolymer. The coatings implement multi-functions, such as inhibiting microbe growth to ensure food safety and maintain food quality, preventing nutrients oxidation to preserve food quality, providing a physical barrier for diminishing water loss and respiration to extend shelf life, and enhancing phytochemicals delivery and body absorption to improve health.

With successful demonstration of these functions, the present studies move one step closer to the new concept of edible coated FCFVs as nutraceutical foods.

### EGCG NPs

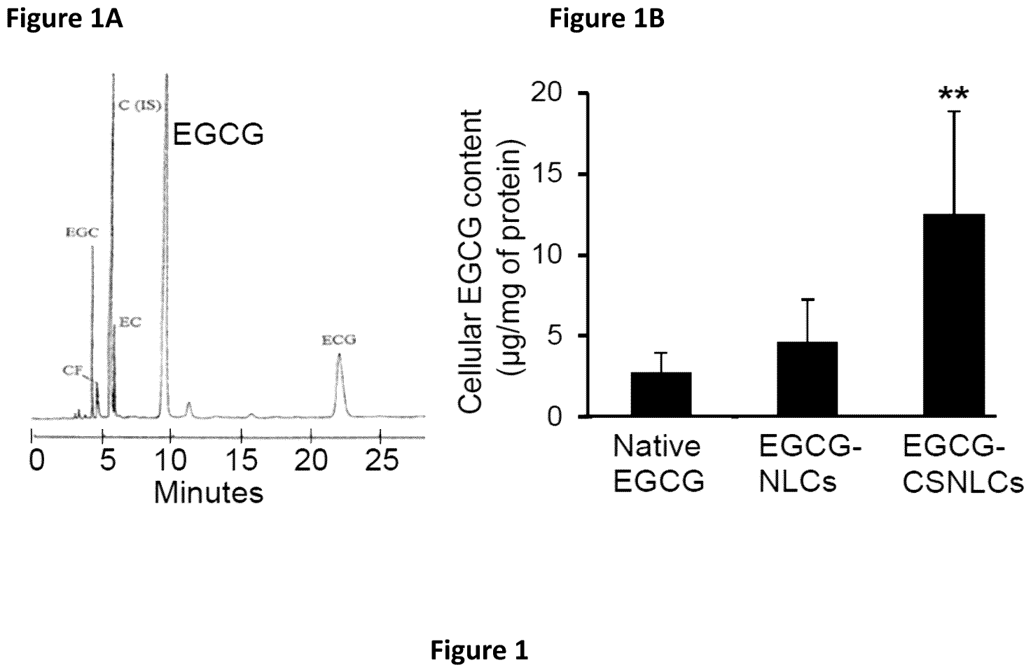

EGCG encapsulated NLCs and CSNLCs were prepared using a phase inversion-based process (Nishiyama N, Nat Nanotechnol. 2007; 2:203-204). They consisted of EGCG, α-tocopherol acetate, soy phospholipids, ethoxylated stearic and oleic acid ester, and CS. They were spherical in shape with mean particle size of 46.3 and 53.5 nm, and polydispersity index (PDI) of 0.25. The zeta potential of NLCs and CSNLCs was-12.6 and +31.4 mV, respectively. EGCG was detected using a high-performance liquid chromatography (HPLC) system (FIG. 1A). EGCG encapsulation efficiency and loading capacity in CSNLCs were about 95% and 3%, respectively. CSNLC significantly enhanced EGCG uptake by mouse macrophages (FIG. 1B).

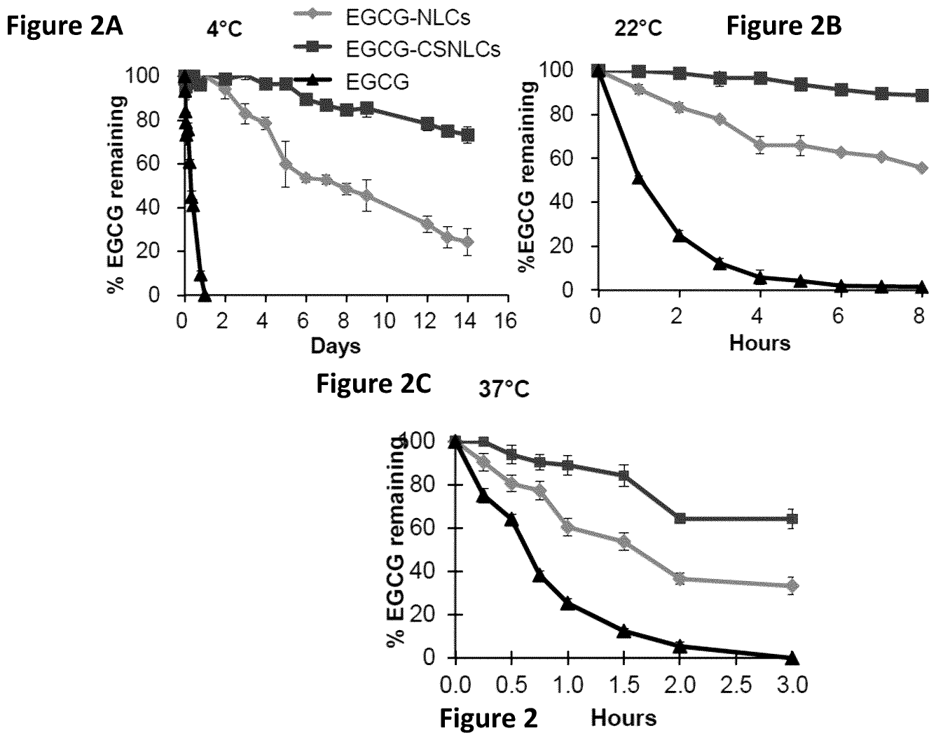

Stabilities of native EGCG, NLCs, and CSNLCs were determined. At neutral pH (7.4), native EGCG was unstable and completely degraded after 1 day, 6 hours, and 3 hours at 4° C., 25° C., and 37° C., respectively (FIG. 2). The degradation rate of EGCG in NLCs and CSNLCs were much slower. After 2 hours incubation, NLCs and CSNLCs were 7 and 12 times more stable than native EGCG, respectively.

### RES NPS

RES encapsulated NLCs (RES-NLCs) were prepared using a mixture containing RES, soy phosphatidylcholine, Kolliphor® HS15, and (+)-α-tocopherol acetate (Zu Y, Colloids Surf B Biointerfaces. 2018; 164:414-423). The RES-NLCs had a vitamin E hydrophobic core, in which RES was loaded and they were spherical. The average particle size was around 140 nm, zeta potentials of-19 mV. The encapsulation efficiency and loading capacity of RES-NLCs were 96.5% and 28.5%, respectively. RES in NLCs had 100-fold higher aqueous solubility than native RES. Native RES showed a burst release phenomenon, while RES-NLCs exhibited a sustained release behavior. RES-NLCs also enhanced the chemical stability of RES

### Quercetin NPs

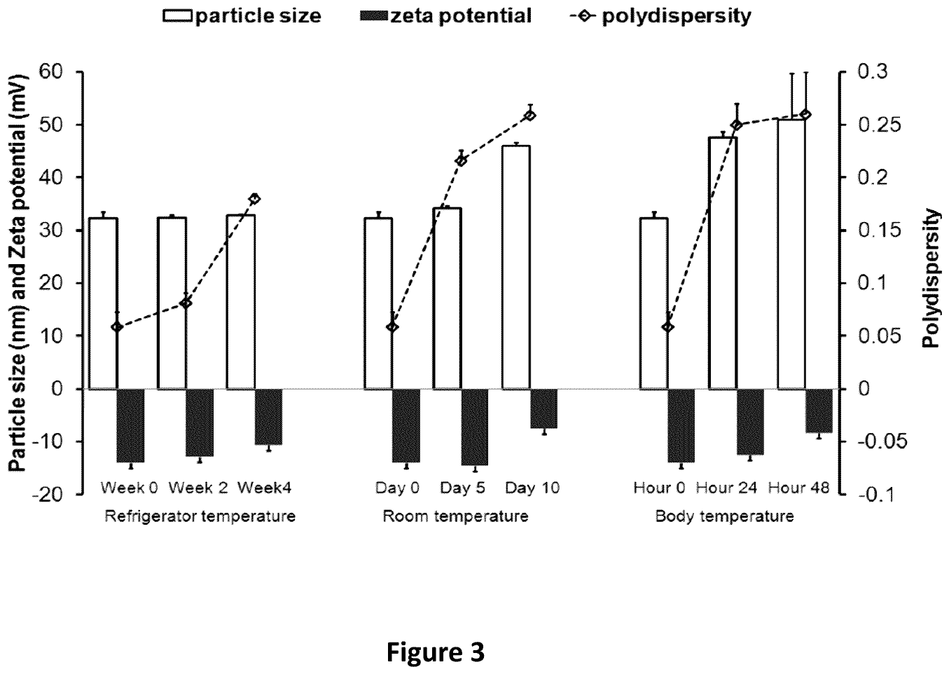

Q-NLCs were prepared using a phase inversion-based process (Sun M, Colloids Surf B Biointerfaces. 2014; 113:15-24). The Q-NLCs consist of Q, vitamin E acetate, soy phosphatidylcholine, PEG, surfactant, and water. Q-NLCs were about 30 nm in diameter with a polydispersity index of 0.059. Nanoencapsulation dramatically increased Q solubility in 1×PBS (Sun M, Colloids Surf B Biointerfaces. 2014; 113:15-24). When 20 mg of Q were encapsulated into NLCs, the Q-NLCs were completely dissolved in 10 mL of 1×PBS (equivalent to 2 g/L) at room temperature, showing a translucent state with visible opalescence. The 20 mg of free Q were not able to be dissolved into the same amount of 1×PBS at the same temperature (Sun M, Colloids Surf B Biointerfaces. 2014; 113:15-24). Q-NLCs were extremely stable at both 4° C. and 22° C. (FIG. 3). The size and PI were increased, and zeta potential was decreased at 37° C.

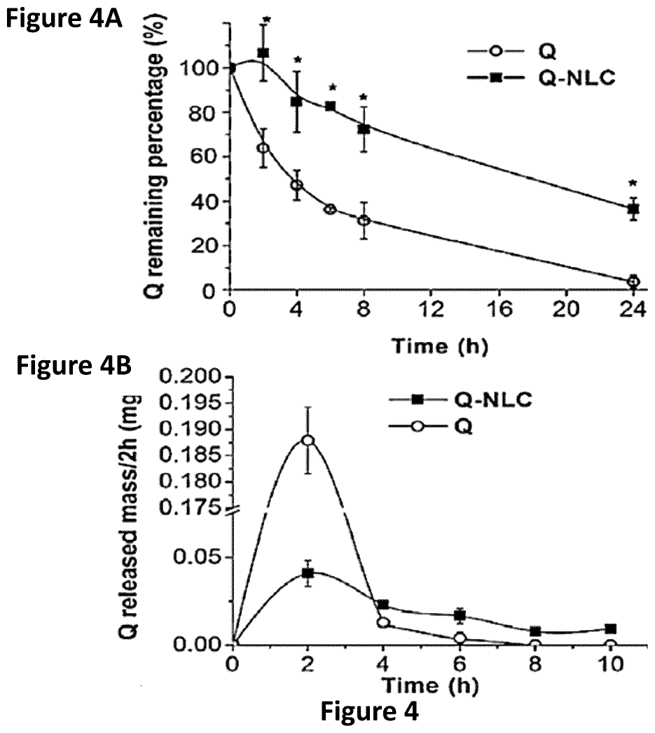

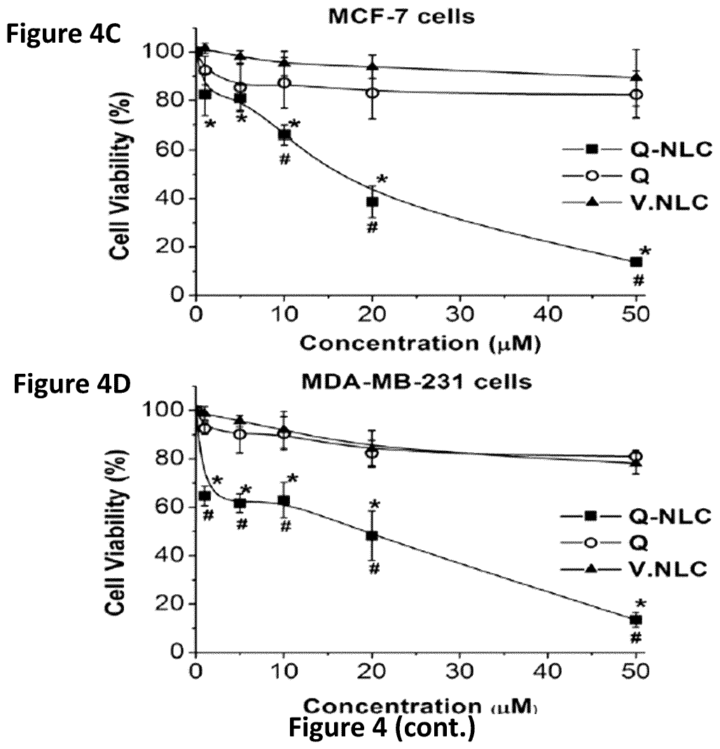

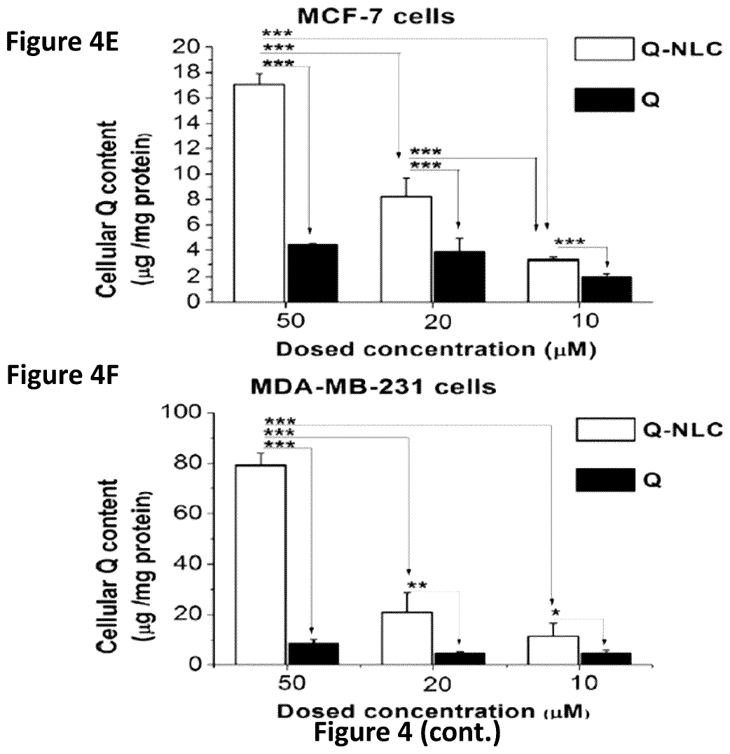

Q-NLCs had significantly increased Q stability (FIG. 4A). When measuring release pattern using a dialysis method, native Q exhibited a burst release pattern, but Q-NLCs exhibited sustained release profile (FIG. 4B). In the dose-dependent cytotoxicity study, MCF-7 or MDA-MB-231 cells were treated with native Q, void NLC (V-NLCs), and Q-NLCs for 48 h. Only Q-NLC demonstrated a distinct dose-dependent inhibitory effect on the viability of both MCF-7 and MDA-MB-231 cells (FIG. 4C and FIG. 4D). As Q-NLCs concentrations increased, the viability of both MCF-7 and MDA-MB-231 cells was proportionally decreased. Q-NLCs significantly increased intracellular Q concentrations compared to native Q (p<0.001). As Q-NLC concentrations were increased, the cellular Q concentrations were proportionally increased in both MCF-7 and MDA-MB-231 cells (FIG. 4E and FIG. 4F).

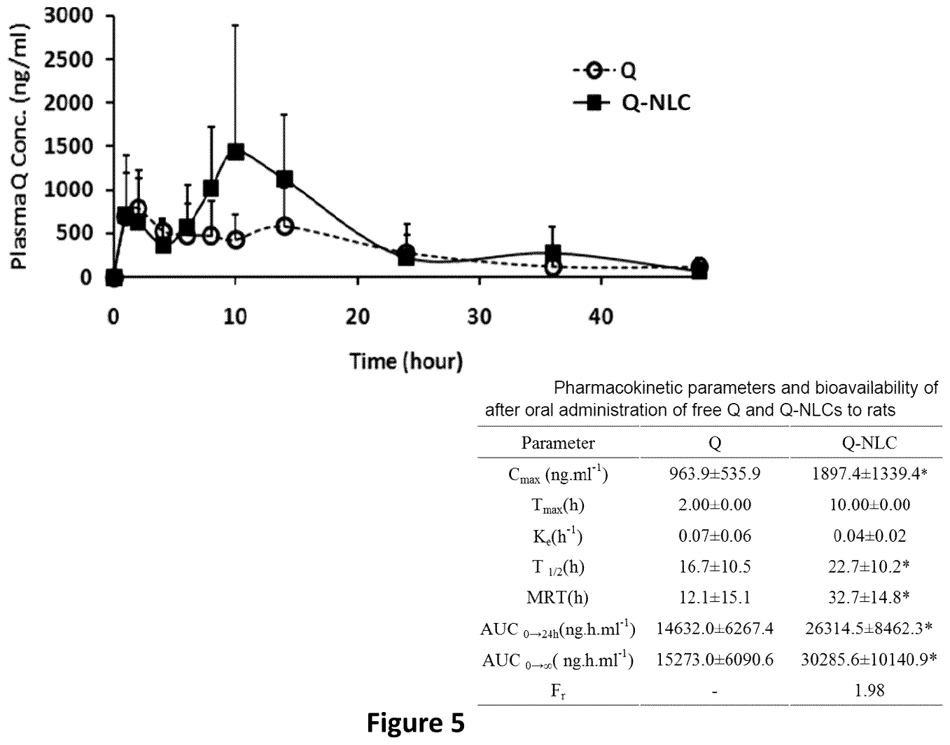

Furthermore, the stability of free Q and Q-NLCs were measured in the simulated gastric solution (pH=1.2) and simulated intestinal solutions (pH=6.8 and pH=7.4). Q-NLCs had significantly higher stability than free Q in those solutions. Male Sprague-Dawley rats (n=3 per group) weighing 200-250 g were purchased pre-cannulated from Harlan Laboratories (Indianapolis, IN). The rounded tips catheters were surgically implanted into the jugular vein of the rats with the benefits of multiple blood draw. Free Q and Q-NLCs were given to male SD rats at dose 50 mg/kg body weight. Blood was collected via the catheters at hour 0, 1, 2, 3, 4, 6, 8, 10, 14, 24, 36, and 48 into heparinized tubes. Blood Q concentrations were measured using a HPLC system (FIG. 5). Pharmacokinetic parameters were determined on each individual set of data by using a non-compartmental model (Song I, Eur J Clin Pharmacol. 2016; 72:665-670). Maximum plasma concentration (Cₘₐₓ) and time of maximum plasma concentration (Tₘₐₓ) were obtained from direct blood Q concentration measurement, and the area under the concentration-time curve from time zero to time 24 h (AUC_{0-24 h}) was calculated using the trapezoidal method (Xu H, J Control Release. 2009; 140:61-68). The area under the plasma concentration-time curve from time zero to infinity (AUC _{0→∞}) was calculated as AUC_{0-24 h}+Cₜ/Kₑ, where Cₜ was the Q concentration observed at last time, and Kₑ is the apparent elimination rate constant obtained from the terminal slope of the individual plasma concentration-time curves after logarithmic transformation of the plasma concentration values and application of linear regression. The relative bioavailability Fr at infinity was calculated as Fᵣ=AUC_{Q-NLC, 0→0}/AUC_{Q, 0→∞}. The half-life (t_{1/2})=0.693/k. The mean residence time (MRT)=1/k. Q-NLCs has about a 2-fold increase in blood Q concentrations and its relative bioavailability as compared to free Q (FIG. 5).

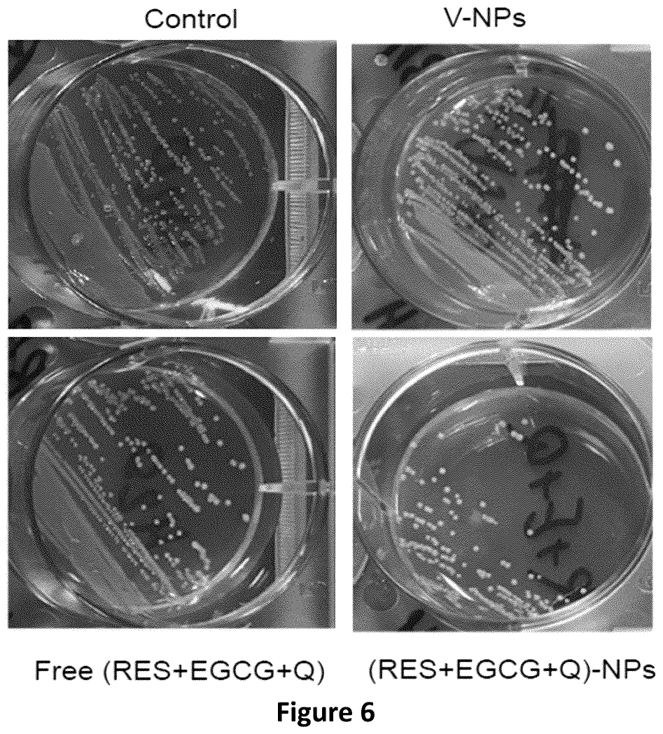

Furthermore, the antimicrobial activity of phytochemical NPs was tested using Listeria monocytogenes (ATCC 15313) bacterial model. After cultivation, the bacterial, mixing with V-NPs, free (RES+EGCG+Q), or (RES+EGCG+Q)-NPs containing 33 μM RES, 33 μM EGCG, and 33 μM Q, were inoculated on Brain Heart Infusion Agar. After incubating at 37° C. for 16 hours, FIG. 6 qualitatively showed that (RES+EGCG+Q)-loaded NPs were much more effective than their free chemicals in inhibiting the growth of Listeria monocytogenes.

In summary, the present studies focused on the investigation of following various phytochemicals (i.e., R, Q, C, and EGCG), pathogens (i.e., gram-negative Escherichia coli (Migula) Castellani and Chalmers (ATCC® 43893), Salmonella enterica subsp. enterica (ATCC® 13076™); gram-positive Staphylococcus aureus subsp. aureus Rosenbach (ATCC® 12600™), and Listeria monocytogenes (ATCC® 15313™)), and FCFV models (i.e., one climacteric fruit (apple) and two non-climacteric fruits (cucumber and strawberry)). Depending on experimental outcomes from individual compounds, their proper mixtures were also pursued for potent synergetic effects. Escherichia coli are grown on soybean-Casein Digest Agar; Staphylococcus aureus subsp. aureus Rosenbach and Salmonella enterica subsp. enterica are grown on 3 Nutrient Agar (BD 213000); Listeria monocytogenes are grown on Brain Heart Infusion Agar (BD 211065). All bacteria are cultured at 37° C. All the stock cultures are stored at 4° C. In the study of active coating for FCFV preservation, the native fungi caused food decay are tested.

The materials and methods employed in the present experimental examples are now described.

### Statistical Methods

The data from the present studies are checked for normality and appropriate transformations are performed, when necessary, prior to statistical analysis (SPSS). The results are analyzed by Student's t-test for two groups, and an analysis of variance followed by Tukey's post hoc test are performed to compare multiple-group means. Differences are considered significant at p<0.05. Each data point are measured in triplicates. Each experiment are performed at least three times independently wherever possible.

### Synthesis and Properties Study of Nanoencapsulations and Coating Films

This task focuses on synthesis and basic physiochemical properties characterization of materials.

### Prepare NPs and Coating-Forming Solutions

NLCs are prepared using soy phosphatidylcholine, vitamin E, Kolliphor® EL surfactant, and the four phytochemicals. A novel phase inversion-based process is used. First, oil and aqueous phase are heated to 85° C. and mixed. The mixture is treated with three temperature cycles from 70° C. to 85° C. under magnetic stirring. In the last cycle, when the mixture is cooled to 79° C. (1 to 3° C. lower than the beginning of the phase inversion zone), cold deionized water (0° C.) is added to the mixture. The fast cooling-dilution process results in NLCs formation. Afterward, a slow magnetic stirring is applied to the suspension for 5 minutes. Last, NLCs are incubated with CS solution to form CSNLCs. All steps in the preparation of NLCs and CSNLCs are performed under nitrogen to prevent phytochemical degradation. The NLCs and CSNLCs are isolated by ultracentrifugation or ultrafiltration (Millipore Amicon Ultra-15). NLCs and CSNLCs are resuspended in 1×PBS or coat-forming solutions.

In the NPs, the phytochemical is dissolved in the vitamin E oily phase. Depending on desired firmness of NPs, the composition of vitamin E (α-tocopherol acetate and (+)-α-tocopherol nicotinate) can be tailored to change the melting point and hence the firmness of NPs, which may impact the tasty of coated FCFVs, or the release dynamics of encapsulated phytochemicals.

CS is the biopolymer used in the coating-forming solutions. CS is dissolved in distilled water containing glacial acetic acid as the solvent. A given amount of suspended NLCs is mixed with the CS solution, where NLCs also act as a plasticizer, to attain the coating-forming solution.

### Characterize NPs

The size and morphology of NPs, including both NLCs and CSNLCs, are measured by TEM. The hydrodynamic size and its distribution (polydispersity (PDI)) are measured using a dynamic light scattering (DLS) technique, and surface potential by a zeta potential analyzer. Chemical interactions between constituents are studied using Fourier Transform Infrared (FTIR) spectroscopy and Raman spectroscopy. Small-angle X-ray analysis (SAXS) are used for nanoscale structure features. Differential scanning calorimetry (DSC) is used for thermo-analysis, especially if firm NPs with high-melting-point oily phase is desired.

Concentrations of phytochemicals are detected by using a HPLC system, as described previously (Zu Y, Colloids Surf B Biointerfaces. 2018; 164:414-423; Zu Y, J Control Release. 2021; 333:339-351; Sun M, Colloids Surf B Biointerfaces. 2014; 113:15-24; Chen Z, Journal of the Science of Food and Agriculture. 2001; 81:1034-1038). The total phytochemical concentrations (Cₜₒₜₐₗ) are measured. Non-encapsulated (Free) phytochemicals in NPs are separated from the encapsulated ones using an ultrafiltration method (Millipore Amicon Ultra-15) and measured by the HPLC system (C_{free}). To calculate the loading capacity, a certain volume of NPs are dried using a vacuum freeze-drying system. The weight of dried NPs are expressed as W_{NANO}. The encapsulation efficiency and the loading capacity of phytochemicals in NPs are calculated according to

$$Encapsulation efficiency = ( {C}_{total} - {C}_{free} ) / {C}_{total} \times 100 \%$$

$$Loading capacity = ( {C}_{total} V - {C}_{free} V ) / {W}_{NANO} \times 100 \%$$

The chemical stability is measured by detecting remaining phytochemicals' concentrations at different time points using the HPLC system. The release profile is measured using a dialysis method. Briefly, native phytochemicals and NCs are each put into dialysis bags with Molecular Weight Cut Off (MWCO) 6,000-8,000. The dialysis bags are dipped into the dissolution medium composed of 1×PBS and methanol (9:1, v:v). The dissolution medium is stirred at 200 rpm at 4° C. and 22° C. The dissolution medium is changed completely every 2 hours for the first 24 hours, and every 6 hours for the rest 30 days. The amounts of phytochemicals in the release medium are measured using the above HPLC methods.

### Characterize Coating-Forming Solutions and Freestanding Films

FCFVs are coated with a thin edible film via a dip-coating process. To easily form a conformal and compact barrier coating, the rheology property of the coating solutions is characterized. The mechanical and barrier performance of the films is investigated using freestanding films with a similar thickness to the fruit coating. The freestanding films are prepared by a solution casting process, followed by solvent evaporation. The characterizations include: viscosity of each solution at resting state and as a function of shear rate to investigate the solution's coating-forming performance. 0.5-1% pure CS solution is used as a reference since it has excellent coating-forming ability; contact angle (wettability) with time of each solution on the selected fruit models, and surface tension measurements, and then calculate work of adhesion and spreading coefficient; thickness and topology of fruit coatings and freestanding films using confocal fluorescence microscopy and SEM; transparency and whiteness of the film; FTIR and Raman spectroscopic study of freestanding films to understand chemical interactions between components and the impacts on their mechanical/barrier performance; mechanical properties of freestanding films, including parameters, such as the tensile strength, elastic modulus, elongation at break (fracture strain), and toughness (energy-to-break); oxygen Permeability (OP) and oxygen transmission rate (OTR). The OP of the film is determined according to the ASTM Method D-3985: OP=(OTR×l)/Δp, where ΔP is O₂ partial pressure difference, and l is the film thickness; water vapor permeability (WVP). Following ASTM Method E96-95 and Pratama Y, International Food Research Journal. 2019; 26, after equilibrated at 44% RH (relative humidity) and 20° C. for 24 h, the film specimen is sealed on a 5 cm glass permeation cup filled with distilled water to establish 100% RH condition. The weight of the cup is measured at 30 min intervals. From the measured water vapor trans-mission rate (WVTR), the deduced water vapor pressures inside and outside of the cup at the given RH (p₂ and p₁), WVP can be calculated: WVP=WVTR×(film thickness/(p₂ and p₁)).

### Antimicrobial Activities of NPs

The antimicrobial activity of free phytochemicals and their NPs are determined qualitatively and quantitatively.

Qualitative Inhibition Zone Method: pure phytochemicals and their CSNLCs are added on the top of the agar plates, and the antimicrobial activity are determined using a modified agar diffusion assay at 37° C. for 2 days. Any clear zone on the agar plate indicates growth inhibition of bacteria.

Quantitative Viable Cell Count Method: pure phytochemicals and their CSNLCs are added into individual sterile flasks containing bacteria and its responsive culture medium and cultivated at 30° C. for 16 hours. Then the tubes of bacterial cell culture are centrifuged at 7,000×g at 4° C. for 5 min, decanted, washed with 0.1% peptone, centrifuged again, and get the pellet. The cell pellet is placed into responsive cell culture broth and diluted to 10% of the original broth concentration with sterile distilled water to obtain an inoculum of ≈(1.0-2.5)×10⁶ colony-forming units (CFU)/mL. Then, 100 mL of the inoculum is aseptically added to the flasks containing pure phytochemicals or their CSNLCs. An inoculum of cell suspension in a flask with only medium is used as a control. The flasks are shaken and rotated at 50 rpm and 30° C. Aliquots of 0.1 mL of cell suspension are periodically taken from the flasks, diluted serially with 0.1% peptone solution, and plated in duplicate on corresponding agars. The plates are incubated in an aerobic chamber at 37° C. for 2 days. The number of colonies on each plate are counted and reported as CFU per mL.

### Antioxidant Activities of NPs

Total Antioxidant Capacity of FCFV lysates are determined using Total Antioxidant Capacity Assay Kit (Sigma MAK187). During this assay, Cu2+ ion is converted to Cu+ by small molecules (vitamin C, vitamin E, beta-carotene, etc), enzymes and proteins (GSH reductase, etc.). The concentrations of reduced Cu+ ion is detected with a colorimetric probe, that has a broad absorbance peak at 570 nm. Trolox, a water-soluble vitamin E analog, is used as an antioxidant standard.

DPPH Scavenging Activity: The 1,1-diphenyl-2-picrylhydrazyl radical (DPPH) is dissolved into ethanol to make 120 μM concentration. Tris-HCl is mixed with this DPPH solution at equal volume. An aliquot of NP solutions or FCFV extract (10 μL) is added into 195 μL of DPPH Tris HCl solution in the microplate. After incubating at dark for 30 minutes, absorbance is measured at 517 nm using a plate reader. Trolox is used as a standard (Shavandi A, Food chemistry. 2017; 227:194-201).

ABTS Scavenging Activity: The 2,2′-azino-bis(3-ethylbenzthiazoline-6-sulfonic acid) (ABTS) is dissolved into deionized water to make 7.4 mM solution, and potassium persulfate is dissolved into deionized water to make 2.6 mM solution. After mixing then at the equal volume, the mixture is incubated at room temperature for 12 hours in the dark. The mixture solution is then diluted 60 times by adding methanol to make the final ABTS solution. NP solutions or FCFV lysates (15 μL) are added into 235 μL of the ABTS solution in the microplate and incubated for 2 hours in the dark. The absorbance is measured at 734 nm using a plate reader. Trolox is used as a standard.

### Multifunctional Nutraceutical Coatings for FCFV Preservation

Fresh and high-quality model fruits are selected. Fruits are selected for uniformity, shape, color, and size. After cutting, they are randomly distributed into groups. For each treatment, samples are dipped twice in fresh coating solutions for 1 min to assure the uniformity of the coating on the whole surface. For each treatment, three replicates are used. They are then placed in punnets, sealed, and stored at 4° C. and 90% relative humidity for progressive assessments.

### Nutraceutical Coatings for Preservation

Weight, color, firmness, ethylene production and respiration rate are determined daily. At the end of the experiment, total soluble solids concentration (TSS), titratable acidity (TA), sugar and organic acid composition and 1-aminocyclopropane-1-carboxylic acid concentration (ACC; free, conjugated and total) are analyzed.

Weight Loss Measurement. Weight loss data are calculated from the measured initially weight following treatment and the weight at subsequent days. The weight loss percentage as a function of time is calculated.

Fruit Firmness. The firmness of fruits is determined following Hietaranta T, HortTechnology. 1999; 9:103-105 by using a tensile testing instrument. With A 100-N load cell, the maximum penetration force is determined using a 1-mm-diameter metal probe. The penetration depth is 5 mm, and the speed is 5 mm/s. The firmness is reported as the maximum peak force (N).

Color. Color is determined using a Chroma Meter, expressed as L* parameter and hue angle h*.

Ethylene Production and Respiration Rate. Ethylene production and respiration rate is determined by gas chromatography and measured every 24 hours after treatment. To measure respiration rate and ethylene production, sam-ples are weighted before and after sealed in a 2.4 L glass chamber for 1 h. 1 mL of gas is sampled with a syringe, injected and analyzed by gas chromatography81.

Total soluble solids concentration (TSS) and titratable acidity (TA). TSS is determined using a digital refractometer. The juice pH is measured using a pH meter. The soluble solid content (SSC) was measured using a digital refractometer. TA is determined potentiometric titration with 0.1 mol L-1 NaOH up to pH 8.1, using 1 mL of diluted juice in 25 ml distilled H2O. TA is expressed as grams of citric acid equivalent per 100 g of fruit (Robledo N, Food and Bioprocess Technology. 2018; 11:1566-1574).

Fungal Decay. Counts of mold and yeast is measured at given days during storage. 10 g sample is taken out from each tray in aseptic conditions and placed in a sterile plastic bag.

Then, 90 mL of sterile saline peptone water is added and homogenized in a blender. The solutions are diluted. 0.1 mL solution is spread over prepared chloramphenicol glucose agar and incubated at 25° C. for 3 days to determine counts of molds and yeasts (Salvia-Trujillo L, Postharvest Biology and Technology. 2015; 105:8-16). The growth of molds and yeasts is fitted using models, such as a Gompertz model (Mckellar R C and Lu X. Modeling microbial responses in food: CRC press; 2003): log₁₀X(d)=A+Cexp(−exp(−B(d−μ))).

### Nutrition Value Assessment

At given day intervals, nutrients including vitamin C, K, Bs are extracted and detected using a UHPLC system. Vitamin K1 is determined using a C18 column. The mobile phase is be acetonitrile/di-chloromethane/methanol (60:20:20, v/v/v), the flow rate is1 mL/min, and the detection wavelength is 248 nm (Otles S, Food Chemistry. 2007; 100:1220-1222). Vitamin E is detected using a silica column (5 μm, 4.6× 250 mm) and a florescence detector. The mobile phase is hexane/1,4 dioxane (96:4, v/v), and the flow rate is 2 mL/min. The excitation and emission wavelengths are 296 nm and 325 nm, respectively (Shen C L, BMC Complement Altern Med. 2018; 18:198). Carotenoid content is detected using a C18 column. The mobile phase is nitromethane/2-propanol/ethyl acetate (80:10:10, v/v/). The flow rate is 1 mL/min, The detection wavelength is 440 nm (Sandmann G., Phytochem Anal. 2010; 21:434-437). Ascorbic acid is detected using an Acquity HSS T3 analytical column. The mobile phase is aqueous 0.1% (v/v) formic acid at a flow rate of 250 μL/min. The detection wavelength is 245 nm (Spínola V, Anal Bioanal Chem. 2012; 403:1049-1058). Vitamin B6 is detected using a LC 18 column. The mobile phase is methanol/phosphate buffer (10:90, v/v) and 0.018 M trimethylamine reagent at pH 3.55 that is adjusted with 85% orthophosphoric acid. The flow rate is 1 mL/min. The detection wavelength is 210 nm (Lebiedzińska A, J Chromatogr A. 2007; 1173:71-80).

### Antimicrobial Activities of Nutraceutical Coatings

To access the bactericidal effect of nutraceutical coatings on fruits, the selected bacterial model is inoculated on the fresh-cut fruits prior to forming the nutraceutical coating. After incubation, a given amount of the bacterial inoculum is aseptically spread on FCFVs homogeneously to get a high initial bacterial load. These samples are then coated with the nutraceutical coating. Bacterial counts are determined at the given day intervals. 10 g of FCFVs is taken from each tray, placed in a sterile plastic bag, and mixed with 90 mL of saline peptone water using a blender. After serial dilutions, they are spread on agar plates, which are then incubated for counting.

### Bioavailability, Bioactivities, and Safety of Edible Coatings in Research Mice

If the selected phytochemicals are sufficiently taken in via NP delivery, the effects is apparent in C57/BL6J mice (a commonly used high-fat diet induced obesity animal model), in terms of increased their bioavailability, reduced inflammation, body weight and fat mass, and improved blood lipid profile and glucose homeostasis. Since these NPs are composed of biocompatible and biodegradable components, no associated toxicities to organs or tissues are expected. Routine necropsy, blood test, and histological examination are performed for the liver, heart, lung, kidneys, brain, spleen, and others on half of the study mice in the study.

### Pharmacokinetics and Bioavailability

Seventy-two C57BL/6J mice are purchased from The Jackson's Laboratory. Free phytochemicals (treatment 1), phytochemical-CSNLCs (treatment 2), and coating composition (phytochemical-CSNLCs mixed with chitosan) (treatment 3), all dissolved in saline, are given to the mice via oral gavage. Twenty-four mice in each treatment group are divided into 8 subgroups at random. Eight subgroups are designed for eight sampling points (1, 2, 4, 8, 12, 24, 48, and 72 hours after oral administration). Three mice in one subgroup at each time point are humanely sacrificed after their blood is drawn from the abdominal vein. Plasma is obtained by blood centrifugation at 1,500×g at 4° C. for 25 min, then aliquoted, frozen, and stored at −80° C. until analysis. The liver, kidneys, adipose tissue, and gastrointestinal (GI) tract are excised and washed with ice-cold saline, weighed, and stored at −80° C. until analysis. The concentrations of phytochemicals in plasma and tissues are determined using the Agilent 1290 UHPLC system. Compartment analysis is used to analyze the data using the standard practical pharmacokinetic program version 97. The following pharmacokinetic parameters of blood, heart, liver, lungs, kidneys, spleen, brain, skeletal muscle, and GI tract are obtained: area under the concentration-time curve (AUC), half-life (t1/2), total plasma or tissue clearance (Cls), apparent distribution volume (Vd), and rate constant of elimination phase (B).

### Anti-Obesity Bioactivities

Eighty male C57/BL6J mice (Jackson Laboratory) are housed at the animal facility of Arizona State University. After 1-week of acclimation, they are weighed and randomly assigned to one of six groups (16 mice per group). C57/BL6J mice are fed a high fat diet (fat providing 45% of energy) and receive one of the following six treatments via mixing into the diet everyday: water (treatment 1), free phytochemicals (treatment 2), V-CSNLCs (treatment 3), phytochemical-CSNLCs (treatment 4), or coating composition (phytochemical-CSNLCs mixed with chitosan) (treatment 5).

They are allowed to drink and eat the high fat diet ad libitum. Mice receive treatments for 8 weeks. Food intake and body weight are measured weekly. After 8 weeks, mice are fasted (6 hours), humanely sacrificed, and complete necropsies are performed. Blood is collected from the abdominal artery under anesthesia, liver, lung, spleen, skeletal muscle, subcutaneous white adipose tissue (WAT), visceral WAT (mesenteric WAT, retroperitoneal WAT, perirenal WAT, and perigonadal WAT)), brown adipose tissue (BAT) (interscapular BAT, and mediastinal BAT), kidneys, brain, heart, and GI tract of each mouse are collected, measured, weighed, and described in detail. Each tissue is cut into 3 pieces to be immediately frozen in liquid nitrogen followed by storage at-80° C.; embedded in OCT compound; and fixed in 4% paraformaldehyde (for histology).

Body composition, energy expenditure, and body temperature measurement: Body composition is measured using Echo-MRI at week 0, 4, and 8. Under anesthesia, transponders are implanted into the abdominal cavity of mice one week before the experiment. Core body temperature is measured for 24 hours at day 0, 7, 14, 21, 28, 35, 42, 49, and 56. Meanwhile, mice are put into metabolic cages for 24 hours on the same days. Respiratory quotient (RQ) and total energy expenditure are calculated as in Weir (Weir J B., J Physiol. 1949; 109:1-9.).

Measurement of phytochemical concentrations in blood and tissues: Tissue samples (200 mg) are homogenized in 1λPBS containing 0.5% ascorbic acid and 0.005% of ethylenediaminetet-raacetic acid (EDTA). Phytochemical extraction is conducted as described in Zu Y, Colloids Surf B Biointerfaces. 2018; 164:414-423; Zu Y, J Control Release. 2021; 333:339-351; Sun M, Colloids Surf B Biointerfaces. 2014; 113:15-24; Chen Z, Journal of the Science of Food and Agriculture. 2001; 81:1034-1038. Dried phytochemicals are dissolved in ethyl acetate and transferred into HPLC vials. After drying under nitrogen, they are resuspended with mobile phases and measured using a HPLC or LC/MS system as described previously. After delipidated tissues are completely dried, they are dissolved into 1N NaOH. Tissue protein are determined using a bicinchoninic acid (BCA) kit. Tissue phytochemical concentrations are expressed as μg of phytochemical per mg of protein.

Measurement of plasma lipid profile: On the sacrifice day (week 8), blood is collected and plasma concentrations of free fatty acids, triglycerides, total cholesterol (TC), low-density lipo-protein (LDL)-C, and high-density lipoprotein (HDL)-C are measured by an enzymatic method as described previously (Wang S, Atherosclerosis. 2009; 204:147-55). Non HDL-C=TC-HDL-C.

Measurement of blood glucose and insulin concentrations and insulin resistance: Mice are fasted for 6 hours at weeks 0, 4, and 8. Fasting blood is collected from tail veins at week 0 and 6 and from the abdominal vein at week 12. Plasma is collected by blood centrifugation at 2,000×g at 4° C. for 20 minutes. Blood glucose and insulin (Millipore) concentrations are measured using commercial kits. The homeostasis model assessment of insulin resistance (HOMA-IR) is calculated using fasting blood glucose and insulin concentrations. The following calculation is widely used and accepted to estimate insulin resistance in humans and animal models:

HOMA-IR=fasting glucose (mmol/L)×fasting insulin (mU/L)/22.5

Glucose tolerance tests (GTT) and insulin tolerance tests (ITT) are performed at the beginning and at end of treatments to assess whole body glucose and insulin tolerance. For GTT, mice are fasted for 6 hours and then injected intraperitoneally with glucose at dose 1 mg/kg body weight. Blood is collected, and blood glucose is measured with a glucometer from tail vein blood at 0, 15, 30, 60, 90, and 120 min post injection. To measure ITT, mice are fasted for 6 hours and then injected intraperitoneally with insulin at dose 0.75 U/kg body weight. Blood is collected, and blood insulin is measured as above at 0, 15, 30, 45, and 60 min post injection.

Plasma and tissue concentrations of inflammatory and anti-inflammatory factors, adiponectin and leptin: Tissue is homogenized. After centrifuging, the supernatant is collected. Concentrations of IL-la, IL-1B, IL-6, IL-10, IL-12p70, IL-17A, IL-23, IL-27, MCP-1, IFN-β, IFN-γ, TNF-α, and GM-CSF in the blood, aorta, liver, and WAT are measured using a LEGENDplex™ mouse inflammation panel kit and flow cytometry. Adiponectin and leptin are determined using the MesoScale Multiplex and ELISA kits (R&D Systems) (Wang S, Atherosclerosis. 2009; 204:147-55).

### Safety Evaluation

Complete necropsies are performed on the day of sacrifice day after treatment. This includes full gross and microscopic evaluation of all organ-systems for all rats. Blood from all rats is used for hematology and clinical chemistry, especially liver panel including alanine transaminase, aspartate transaminase, alkaline phosphatase, albumin and total protein, bilirubin, gamma-glutamyltransferase, L-lactate dehydrogenase, glutamate dehydrogenase, renal panel including urea/blood urea nitrogen (BUN), and creatinine.

After terminal exsanguination under isoflurane, heart, liver, lungs, kidneys, skeletal muscle, brain, a draining lymph node, and spleen of each mouse are collected, measured, weighed and described in detail. They are fixed, embedded, sectioned, and stained for histological examination and evaluation.

Histological analysis: Liver (the potential site of phytochemical metabolism and excretion of lipophilic metabolites), kidneys (the potential site of excretion for hydrophilic metabolites), skeletal muscle (the site for determination of direct and indirect drug toxicity), brain (the site for determination of direct and indirect drug toxicity), aorta and heart (circulation site of phytochemicals) are fixed in 10% neutral buffered formalin. Fixed tissues are embedded, sectioned in 4 μm slices, and stained by hematoxylineosin. Slices are also stained by trichrome to evaluate the appearance of fibrosis in chronic toxicity.

Lipid staining and evaluation: Kidneys, liver, cardiac, and skeletal muscle are examined for neutral lipids. A separate section of the organ is flash-frozen in liquid nitrogen-cooled isobutene and mounted using OCT compound. The frozen section is sectioned in 8 μm slices. They are stained using Oil-Red-O (for neutral triglycerides and lipids). Using Oil-Red-O, the presence and amounts of neutral lipids in each of the tissues are evaluated and semi-quantitatively score. This allows to evaluate and quantify the appearance of micro-vesicular or macro-vesicular fatty change in acute toxicity (neutral lipids).

### Example 2: Chitosan-Coated Resveratrol and Quercetin Nanoparticles: Synergistical Anti-bacterial Activities and Improvement of Nutrition Value of Fruits

Many natural phytochemicals, with their antioxidant, antimicrobial, antigenotoxic, anti-inflammatory, and antidiabetic functions, are attractive healthy supplements. They are, however, easily degraded during storage and metabolized in the gastrointestinal tract, and therefore bio-unavailable after consumption. Furthermore, most people do not consciously take supplements unless health has become an issue. As such, a good design of nutrition supplements should leverage popular foods or fruits as their carrier. The present studies focused on nano-encapsulation of phytochemicals to overcome these issues and improve the stability, bioavailability, and bioactivities of phytochemicals.

On a second frontier, active packaging based on natural phytochemicals is of great importance in food preservation against quality degradation, spoilage, and waste generation, and in food safety against foodborne outbreaks. As such, the present studies also focused on the development of edible coatings on foods that contained nano-encapsulated phytochemicals to actively protect foods as part of the package function, and to effectively delivery and enhance body absorption of otherwise easily degraded nutrients with much-added nutraceutical values, thus balancing the cost-value of actively packaged foods as effective nutraceutical foods for largescale market acceptance.

More specifically, the present studies applied a holistic approach by designing a nano-encapsulated phytochemicals-based multifunctional coating on foods as nutraceutical foods to solve the above described problems. As a protective coating, it provided a physical barrier and slow-release of the nano-encapsulated antioxidation and antimicrobial compounds to maintain the quality and safety of foods, extending their shelf life and reducing food waste. As an edible coating, it also provided a carrier for nutraceutical supplements in promoting health. The nano-encapsulation ensured effective delivery and enhanced body absorption of these compounds before they were degraded in storage and in the gastrointestinal tract. The edible coating can also contain other natural flavors to enhance or even modify the taste of the protected fresh produce itself to create new flavor foods. Accordingly, the present studies describe the incorporation of nano-encapsulated phytochemicals into biocompatible and biodegradable food packaging materials for enhancing both shelf life (antimicrobial activity) and nutrition value (increasing bioavailability) of foods. By enhancing food safety, reducing food waste, and providing bioactive nutritional supplements, the new fresh produce balances their price and value, overcome the cost barrier for consumer acceptance, and becomes a popular health diet.

Nanotechnology in association with biotechnology can develop novel nanomaterials with diverse applications, such as anti-bacterial activity, whereas the extensive use of resveratrol (R) and quercetin (Q) has been limited due to their instability, poor solubility, and low bioavailability. To overcome these limitations and to improve their anti-bacterial effects, the present studies developed resveratrol-(R-) and quercetin-(Q-) based nano-systems. The present study therefore evaluated the characteristics and anti-bacterial activity of resveratrol nanoparticles (R-NPs), quercetin nanoparticles (Q-NPs), and the synergistic anti-bacterial activity of resveratrol and quercetin nanoparticles (RQ-NPs) (Table 2 though Table 5).

TABLE 2
Exemplary results demonstrating that the stability improvement
of R-NPs before and after coating with CS at day 0 and day 1.

| R-nano | | | | | | | | | |
|---|---|---|---|---|---|---|---|---|---|
| Add | Day 0 | | | | | | Day 1 | | |
| chitosan | Before adding CS | | | After adding CS | | | After adding CS | | |
| (mg) | Size | PDI | Zeta | Size | PDI | Zeta | Size | PDI | Zeta |
| 0.03125 | 54.96 | 0.2706 | | 91.62 | 0.1331 | | 74.31 | 0.1191 | |
| 0.0625 | 69.87 | 0.2814 | | 107.00 | 0.1000 | | 83.72 | 0.0800 | |
| 0.125 | 65.20 | 0.3000 | | 97.00 | 0.1545 | | 71.91 | 0.0870 | |
| 0.25 | 64.96 | 0.2722 | | 95.83 | 0.1233 | | 58.84 | 0.1134 | |
| 0.5 | 44.71 | 0.1893 | −1.50 | 88.61 | 0.1984 | 19.47 | 54.82 | 0.1946 | 20.76 |
| 1 | 49.56 | 0.2680 | −1.95 | 93.26 | 0.2374 | 27.67 | 75.84 | 0.1925 | 21.65 |
| 2 | 52.78 | 0.3000 | −5.50 | 86.65 | 0.2184 | 26.82 | 60.08 | 0.2695 | 23.44 |
| 4 | 47.63 | 0.2589 | −3.03 | 88.25 | 0.2486 | 59.44 | 75.40 | 0.2791 | 32.57 |

TABLE 3
Exemplary results demonstrating that the stability improvement
of R-NPs after coating with CS at day 2, day 3, and day 7.

| R-nano | | | | | | | | | |
|---|---|---|---|---|---|---|---|---|---|
| Add | Day 2 | | | Day 3 | | | Day 7 | | |
| chitosan | After adding CS | | | After adding CS | | | After adding CS | | |
| (mg) | Size | PDI | Zeta | Size | PDI | Zeta | Size | PDI | Zeta |
| 0.03125 | precipitation | | | | | | | | |
| 0.0625 | | | | | | | | | |
| 0.125 | | | | | | | | | |
| 0.25 | | | | | | | | | |
| 0.5 | 41.45 | 0.2383 | 35.52 | precipitation | | | | | |
| 1 | 46.52 | 0.2611 | 21.18 | slight precipitation | | | | | |
| 2 | 60.21 | 0.2386 | 22.19 | 58.01 | 0.2265 | 25.59 | 51.5 | 0.2174 | 25.77 |
| 4 | 70.06 | 0.2941 | 40.53 | 77.71 | 0.2667 | 32.86 | 77.46 | 0.2398 | 38.14 |

TABLE 4
Exemplary results demonstrating that the stability improvement of
Q-NPs before and after coating with CS at day 0, day 1, and day 2.

| | Q-nano | | | | | | | | | | | | |
|---|---|---|---|---|---|---|---|---|---|---|---|---|---|
| | Add | Day 0 | | | | | | Day 1 | | | Day 2 | | |
| | chitosan | Before adding CS | | | After adding CS | | | After adding CS | | | After adding CS | | |
| | (mg) | Size | PDI | Size | Size | Size | Zeta | Size | PDI | Zeta | Size | PDI | Zeta |
| | 0.5 | 34.45 | 0.2287 | −5.35 | precipitation | | | | | | | | |
| | 1 | 30.31 | 0.1064 | −4.28 | 57.94 | 0.2279 | 37.58 | 64.90 | 0.2400 | 38.35 | slight precipitation | | |
| | 2 | 36.98 | 0.2298 | −3.51 | precipitation | | | | | | | | |
| Repeat | 1 | 32.15 | 0.1261 | −11.28 | 55.15 | 0.2306 | 17.26 | 31.62 | 0.1272 | 11.82 | 32.5 | 0.1436 | 8.637 |
| | 2 | 32.77 | 0.1643 | −8.811 | 55.91 | 0.2742 | 17.07 | 35.61 | 0.2452 | 10.31 | 38.37 | 0.1638 | 10.77 |

TABLE 5
Exemplary results demonstrating that the stability improvement of
RQ-NPs before and after coating with CS at day 0, day 1, and day 2.

| RQ- | | | | | | | | | | |
|---|---|---|---|---|---|---|---|---|---|---|
| nano | | | | | | | | | | Day 1 |
| Add | Day 0 | | | | | | Day 1 | | | After |
| chitosan | Before adding CS | | | After adding CS | | | After adding CS | | | adding |
| (mg) | Size | PDI | Zeta | Size | PDI | Zeta | Size | PDI | Zeta | CS |
| 2 | 35.77 | 0.1908 | −9.97 | 63.28 | 0.2340 | 27.68 | 45.72 | 0.2396 | 22.66 | slight |
| | | | | | | | | | | precipitation |

In addition, chitosan (CS) is a rich natural polysaccharide and well-studied because of its immense features, including anti-bacterial capacity, biocompatibility, and biodegradability. CS was also reported to be a strong nanoparticle stabilizer. For this reason, the present studies also developed and investigate CS-coated R-NPs, Q-NPs, and RQ-NPs and characterized their improvement. Moreover, the applications of the developed NPs in fruit, such as strawberry, were also studied.

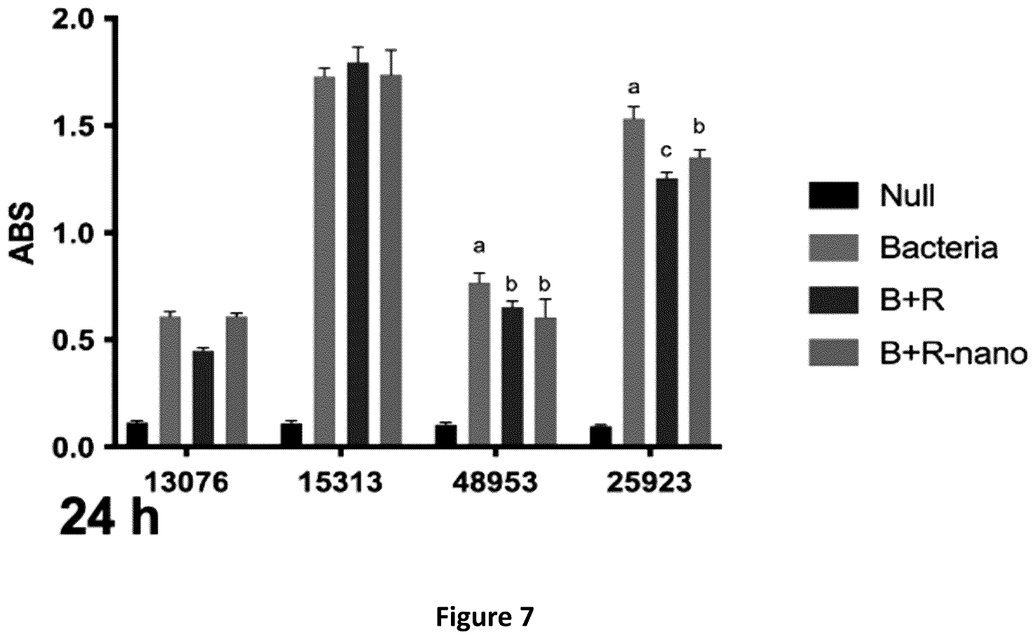

The concentrations of R and Q in the NPs were characterized by HPLC. Three parallel experiments were performed for each sample, the absorbance of bacteria solution treated with different samples was measured by a microplate reader at a wavelength of 600 nm. S. enteritidis (13076), L. monocytogenes (15313), E. coli (48953) and S. aureus (25923) were used in this study and treated with R-NPs, Q-NPs, RQ-NPs, and CS-coated R-NPs, Q-NPs, and RQ-NPs, respectively. Different concentrations of R-NPs and Q-NPs were investigated. Initially, 50 μg/mL R and R-NPs were used. The results showed that 50 μg/mL of R-NPs demonstrated anti-bacterial effects for E. coli (48953) and S. aureus (25923), but not for S. enteritidis (13076) and L. monocytogenes (15313), when compared to the bacterial solutions control group after 24 h of incubation (FIG. 7).

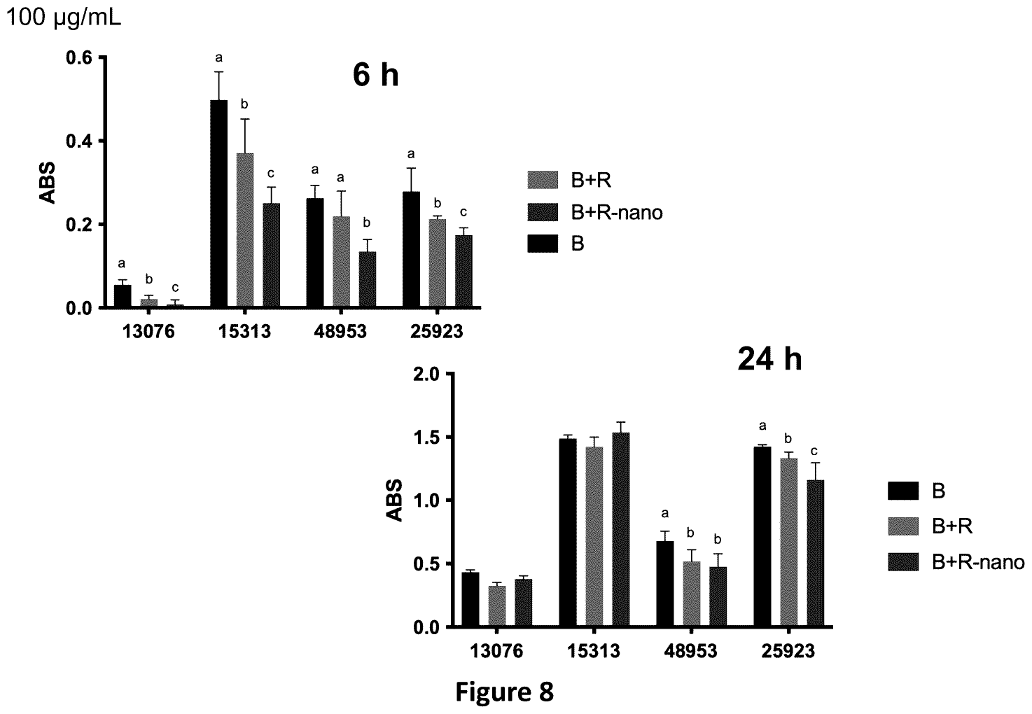

In subsequent studies, 100 g/mL R and R-NPs were used. As shown in FIG. 8, after 6 h of incubation, 100 g/mL R and R-NPs both exhibited significant anti-bacterial effects against the four bacteria strains. Moreover, after 6 h of incubation, 100 μg/mL R-NPs showed significantly better anti-bacterial effects when compared with 100 g/mL free R. However, after 24 h of incubation, both 100 μg/mL R and R-NPs showed anti-bacterial effects against E. coli (48953) and S. aureus (25923), but not S. enteritidis (13076) and L. monocytogenes (15313), which was similar to 50 μg/mL R and R-NPs treatment results (FIG. 7). Colony forming unit (CFU) of the four strains were also counted for further confirmation after 24 h of incubation. For the CFU formation, 100 μg/mL R and R-NPs significantly decreased the growth of E. coli (48953) and S. aureus (25923), but were still not shown in S. enteritidis (13076) and L. monocytogenes (15313). As such, the results indicated that 100 μg/mL R and R-NPs can significantly decreased the growth of E. coli (48953) and S. aureus (25923).

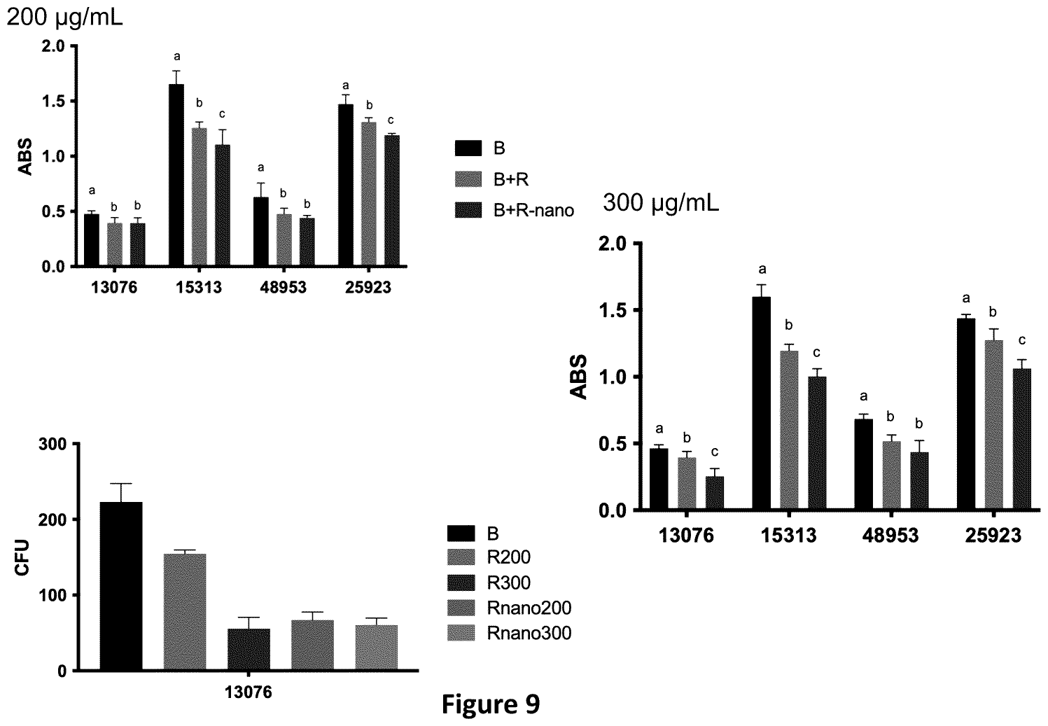

For effective anti-bacterial effect against S. enteritidis (13076) and L. monocytogenes (15313), 200 μg/mL and 300 μg/mL R and R-NPs were used for further investigation. As shown in FIG. 9, 300 μg/mL R and R-NPs treatment exhibited significant enhanced anti-bacterial effects than 200 μg/mL. Furthermore, R-NPs treatment demonstrated better anti-bacterial effects than R. In summary, after 6 h of incubation, 100 μg/mL R and R-NPs both exhibited a significant anti-bacterial effects against the four strains. However, after 8 h of incubation, anti-bacterial effects were not shown in S. enteritidis (13076) using 100 μg/mL R and R-NPs, but were shown in S. enteritidis (13076) using 300 μg/mL R-NPs. CFU of S. enteritidis (13076) was also investigated (FIG. 9). Thus, further studies used ≥300 μg/mL R-NPs for the S. enteritidis (13076), ≥200 μg/mL R-NPs for the L. monocytogenes (15313), and ≥50 μg/mL R-NPs for E. coli (48953) and S. aureus (25923).

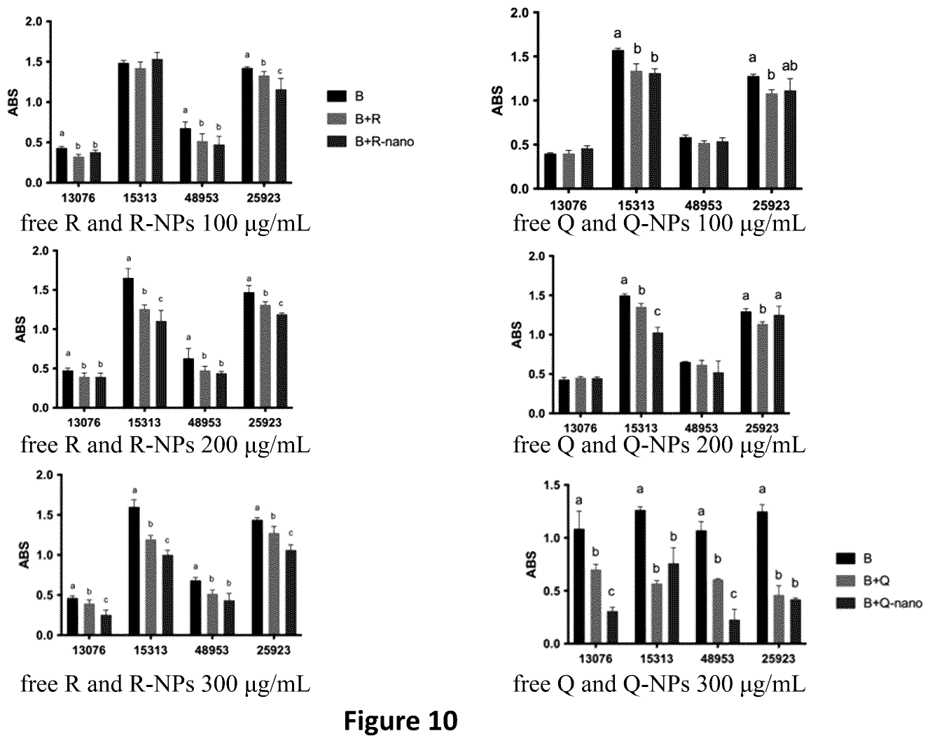

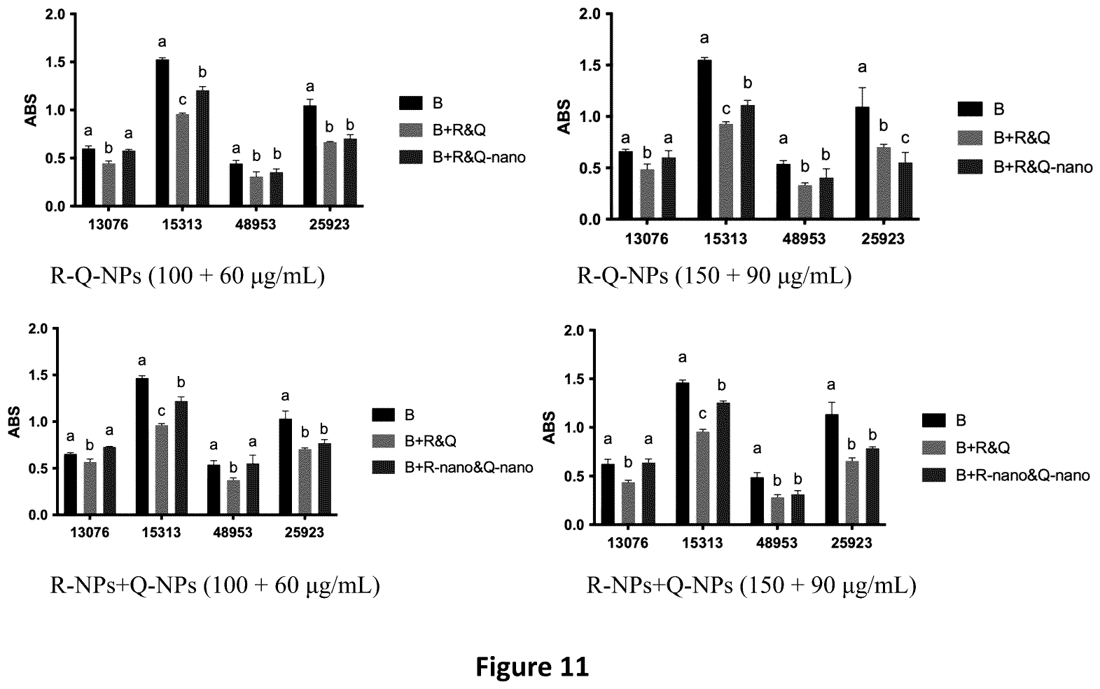

Additional studies demonstrated that 200 g/mL R-NPs and 300 μg/mL Q-NPs inhibited the growth of the Gram-positive and Gram-negative bacteria used in the present study when compared to a control group and free-R or free-Q solutions (FIG. 10). Furthermore, the RQ-NPs and R-NPs+Q-NPs were prepared (FIG. 11). The R concentration in those NPs samples was adjusted to 100 μg/mL and 150 μg/mL before using in the experiment, and the Q concentration in the NPs sample was ˜ 60 μg/mL and 90 μg/mL, respectively. As shown in FIG. 11, the four strains showed same trend under the two concentrations, which also showed better anti-obesity effects when compared to the results using R-NPs and Q-NPs. The anti-bacterial effects were also detected using the combination of R-NPs and Q-NPs. The R-NPs and Q-NPs were prepared individually and added together after adjusting for their concentration. The R-NPs and Q-NPs (150+90 μg/mL) showed a better anti-bacterial effect than R-NPs and Q-NPs (100+60 μg/mL) for E. coli (48953). The RQ-NPs showed a better anti-bacterial effect than R-NPs and Q-NPs, as shown in FIG. 11 for S. aureus (25923) and E. coli (48953). RQ-NPs successfully inhibited the growth of L. monocytogenes (15313), E. coli (48953), and S. aureus (25923), not shown in S. enteritidis (13076). Moreover, a lower concentration of R and Q in RQ-NPs exhibited enhanced anti-bacterial effects.

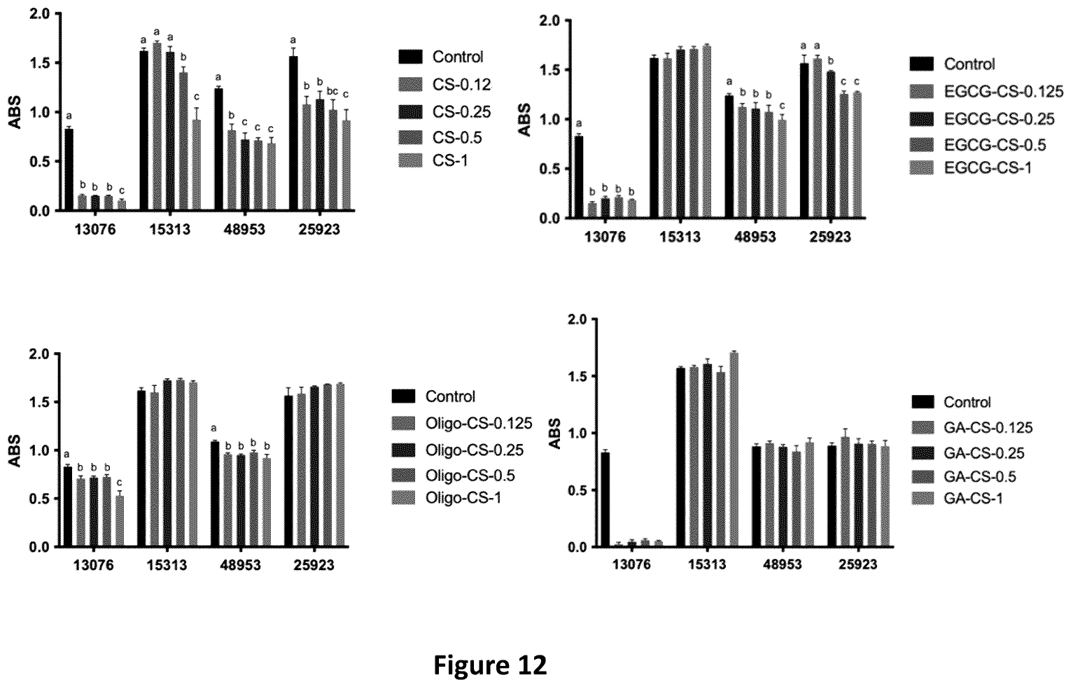

Anti-bacterial effects of CS, EGCG-CS, Oligo-CS, gallic acid-CS (GA-CS) against S. enteritidis (13076), L. monocytogenes (15313), E. coli (48953) and S. aureus (25923) were investigated to determine the best coating component. As shown in FIG. 12, CS was selected to be used to coat with NPs to improve the stability and anti-bacterial effects of NPs. After coated with CS for 30 mins, the size of R-NPs, Q-NPs, and RQ-NPs were all increased and the polydispersity indexes (PDI) of those NPs were inversely changed from negative to positive. Although not bound by any particular theory, it was hypothesized that this change was the reasons for the increased stability and enhanced anti-bacterial effects of CS-coated R-NPs, Q-NPs, and RQ-NPs when compared to non-CS-coated NPs treatment (Table 2 through Table 5). In addition, the CS-coated NPs exhibited synergistical antimicrobial activities, which effectively prolonged the shelf life of strawberries. Strawberries were used for testing whether NPs treatment, especially using CS-coated NPs, maintained the better color of strawberries, enhanced their shelf life, and increased nutrition value of strawberries.

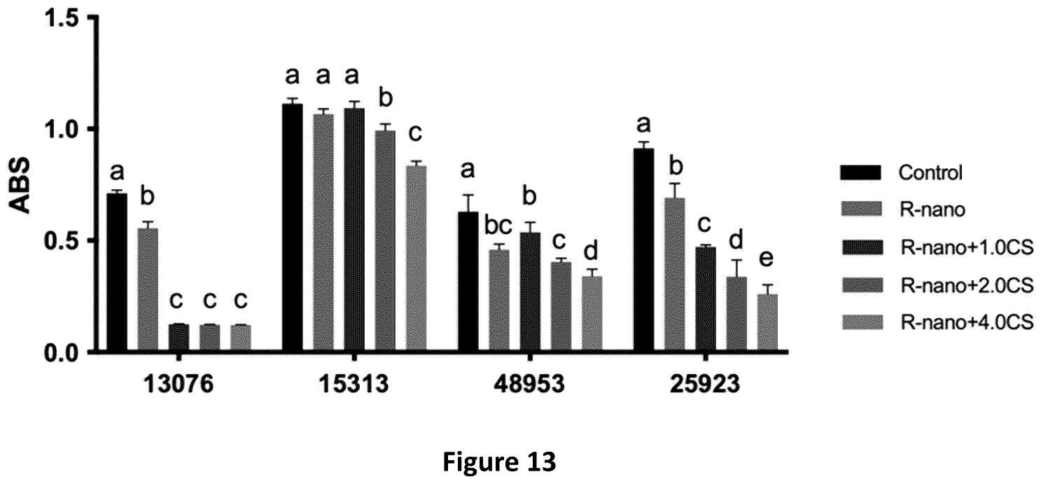

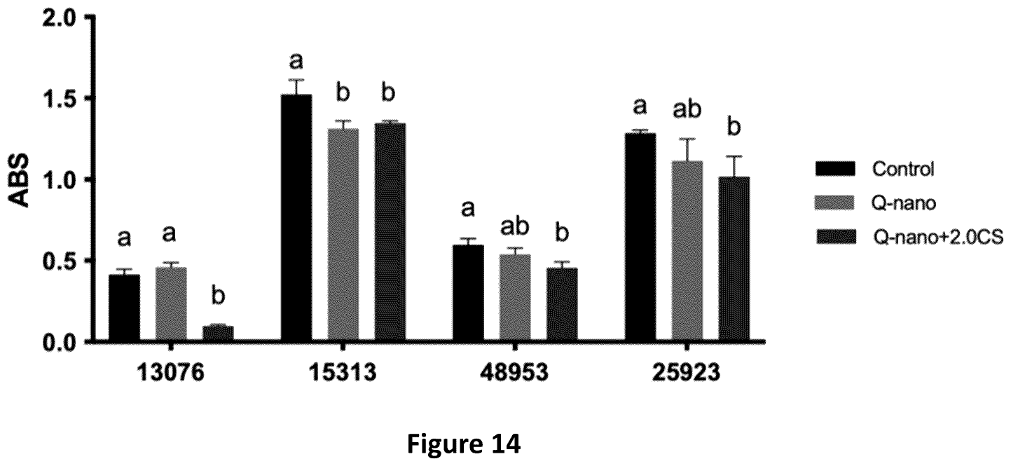

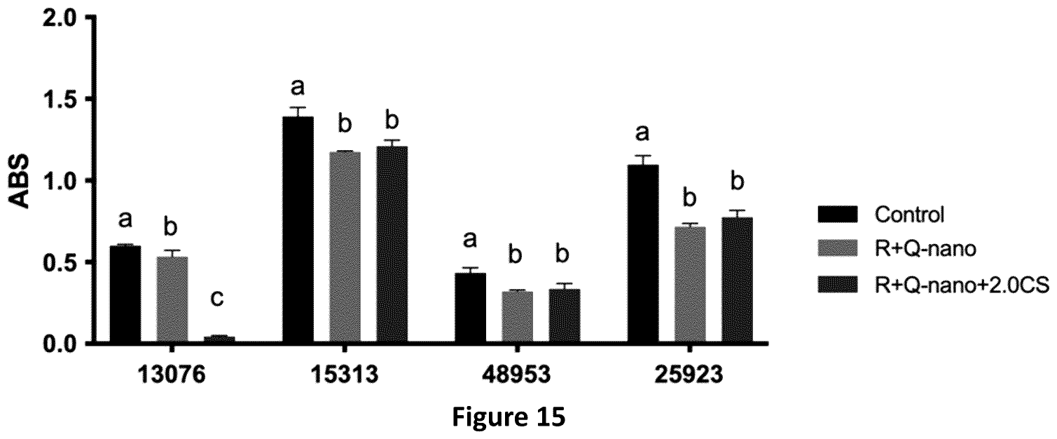

CS-coated RQ-NPs showed a synergistic anti-bacterial activity when compared to R-NPs and Q-NPs (FIG. 13 and FIG. 14). In addition, R-NPs, Q-NPs, and RQ-NPs showed a significant improved stability and anti-bacterial effects after coating with CS, which indicated that CS can be used as a stabilizer in an edible coating material for R-NPs, Q-NPs, and RQ-NPs (FIG. 13 through FIG. 15). Furthermore, treatment of strawberries with the present NPs, especially the CS-coated RQ-NPs, delayed decay of strawberry during storage, indicating their potential implementation in active food packaging.

In summary, nano-encapsulated nutrients are commercially available, but they are used as a dietary supplement. Many people do not like the expensive supplement. Furthermore, the current techniques for increasing shelf life include keeping foods clean, dry, and undamaged. For this reason, the present studies utilized biocompatible and biodegradable food packaging materials, like edible materials, for making food coating/packaging materials. These materials demonstrated antimicrobial activities. The present studies also incorporated nano-encapsulated phytochemicals into the coating/packaging materials. Moreover, the nano-encapsulated phytochemicals synergistically increased antimicrobial activities (for improving shelf life) and enhanced nutrition value of foods by increasing phytochemical bioavailability.

For example, NPs used in the present studies were R-NPs, Q-NPs, and RQ-NPs, and the NPs were also optionally coated with CS. As shown above, RQ-NPs exhibited enhanced antibacterial effects (lower concentration of R and Q in RQ-NPs) and CS coated NPs showed synergistical antimicrobial activity and improved stability of food. Moreover, the nano-encapsulated phytochemicals were incorporated into the coating/packaging materials. The nano-encapsulated phytochemicals synergistically increased antimicrobial activities and enhanced nutrition value of foods by increasing phytochemical bioavailability. As a protective coating, the nano-encapsulated phytochemicals provided a physical barrier and the slow-release of the nano-encapsulated antioxidation and antimicrobial compounds maintained the quality and safety of foods, extending their shelf life and reducing food waste. As an edible coating, the nano-encapsulated phytochemicals provided a carrier for nutraceutical supplements in promoting health. The nano-encapsulation ensured effective delivery and enhanced body absorption of these compounds before they were degraded in storage and in the gastrointestinal tract. The edible coating also contains other natural flavors to enhance or even modify the taste of the protected fresh produce itself to create new flavor foods. By enhancing food safety, reducing food waste, and providing bioactive nutritional supplements, the new fresh produce could balance their price and value, overcome the cost barrier for consumer acceptance and become a popular health diet.

The materials and methods employed in the present experimental examples are now described.

### Preparation of Nanoparticles (NPs)

A mixture composed of 1 mg R, 1.75 mg L-α-phosphatidylcholine (soy PC), 5.5 mg polyethylene glycol 15-hydroxystearate (Kolliphor® HS15), 5.5 mg alpha-tocopherol acetate (αTA) was dissolved in ethanol. After mixing, ethanol was removed using a nitrogen evaporator. The R-nano lipid mixture was then suspended in 1 mL warm deionized water and homogenized and sonicated to get R-NPs.

CS-coated nanoparticles were formed by mixing CS with NPs for 30 mins using magnetic stirrer, or vortexer. The prepared NPs were passed through a 100 kDa Amicon Ultra centrifugal filter (Millipore, Billerico, MA) to eliminate free R, dye, peptide, and/or other compounds.

### Characteristics of Nanoparticles

The particle size and PDI were measured using a Brookhaven BI-MAS particle size analyzer, and the zeta potential was measured using a Zeta PALS analyzer (Brookhaven Corporation, NY). The morphology and size of NPs were determined using a 200 kV Hitachi H-8100 transmission electron microscopy (TEM) instrument (Tokyo, Japan) (Table 1).

TABLE 1
Physical Characteristics of R-NPs, Q-NPs, and RQ-NPs.

| | Particle Size | Zeta Potential | | |
|---|---|---|---|---|
| | (nm) | (mV) | Polydispersity | EE |
| R-NPs | 56.2 ± 8.7 | −3.0 ± 1.5 | 0.268 ± 0.033 | 92% |
| Q-NPs | 33.9 ± 3.3 | −3.9 ± 0.4 | 0.168 ± 0.062 | 91% |
| (R + Q)-NPs | 35.8 ± 1.9 | −9.9 ± 1.0 | 0.191 ± 0.026 | 95% |

R concentrations, encapsulation efficiency, and loading capacity were measured as follows: one volume of R-NPs was dissolved in 9 volumes of methanol, and the total R concentrations (Ctotal) in the R-NPs or RQ-NPs solution were measured using a Shimadzu high performance liquid chromatography (HPLC) system equipped with two LC-20 CE solvent delivery units, a SIL-20 AC HT autosampler, and a SPD-M20A photo diode array (PDA) detector (Shimadzu scientific instruments, Inc., Japan) with a C18 reverse-phase column (Symmetry® C18, 3.5 μm, 4.6×75 mm). The mobile phase was composed of methanol/water/acetic acid (50/50/0.5, v/v/v) with a flow rate of 1 mL/min. The detection wavelength was 310 nm. Free (non-encapsulated) R was separated from nano-encapsulated R using an ultrafiltration method (Millipore Amicon Ultra-15), and its concentrations were measured by the HPLC system (Cfree).

### Anti-Bacterial Assay

S. enteritidis, L. monocytogenes, E. coli and S. aureus were cultured in broth media under constant shaking at 100 rpm at 37° C. to reach the mid exponential growth phase. The number of bacteria was determined by counting the CFU formed on the agar plate. Bacterial suspensions were diluted to approximately 1×10⁴⁻⁵ CFU/mL in broth media. The NPs solution (20 μL) was mixed with 180 μL of bacterial suspension in a 96-well plate, and the experiments were performed in triplicates. The plates were incubated at 37° C. under constant shaking at 100 rpm overnight, and the optical density (OD) at 600 nm was measured on a plate reader.

### Effect on Fruit Quality Parameters

The nanofibrous film was stripped off from tin foil paper, and then placed on the surface and interior of fruit preservation box for film application. The strawberries (6 strawberries in each box) were preserved with NPs with different concentrations. The weight loss of strawberries and their chroma values were measured at 1, 3, and 5 days.

### Example 3: R and Q NPs

The present studies investigated representative NPs, their antibacterial activity, and particularly the results of using strawberries as a testbed to demonstrate the potential of nanoencapsulated R and Q as a protective coating to extend shelf life and preserve nutrient value.

### NPs Synthesis and Properties

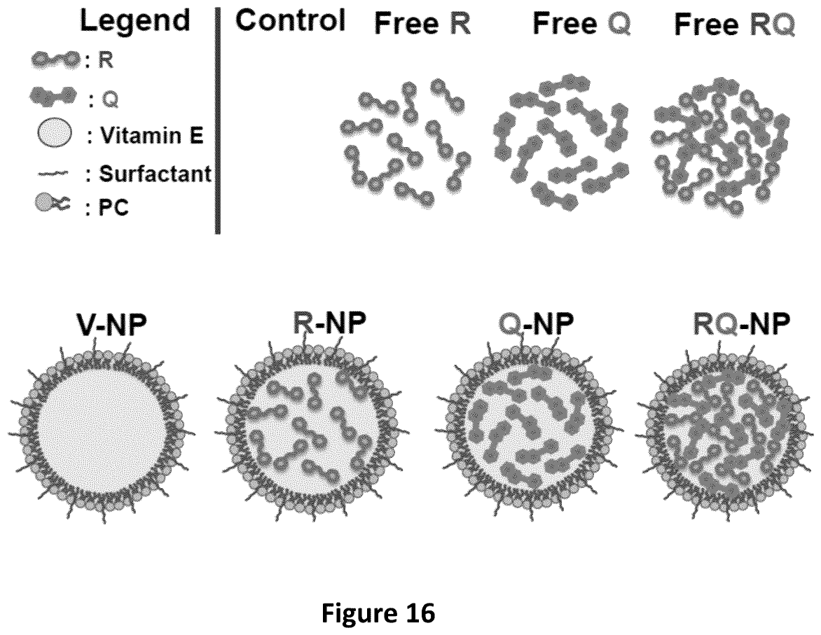

R-NPs, Q-NPs, and R plus Q-NPs (RQ-NPs) were prepared. The NPs were made using a mixture containing soy phosphatidylcholine (PC), (+)-α-tocopherol acetate, α-tocopherol nicotinate, surfactant, and R and/or Q. The void-NPs (V-NPs) are empty NPs without R and Q (FIG. 16). All components were dissolved in ethanol and dried via a nitrogen evaporator. The dried lipid mixture was then suspended in 75° C. deionized water, vortexed, and sonicated for 30-60 seconds.

R-NPs, Q-NPs, and RQ-NPs were spherical, about 30-50 nm in size, their polydispersity index (PDI) was <0.3, and zeta potential was negative. Their encapsulation efficiency was >90%. When 3 mg of R or Q or R+Q was encapsulated, the R-NPs, Q-NPs, and RQ-NPs were completely dissolved in 4 mL of 1×PBS (equivalent to 0.75 g/L) at room temperature (RT), showing a translucent state with visible opalescence. Nanoencapsulation dramatically increased their aqueous solubility by 50 times.

After coating NPs with chitosan (CS), the size of CS-R-NPs, CS-Q-NPs, and CS-RQ-NPs was increased by about 20 nm, PDI was <0.3, and their zeta potentials became positive.

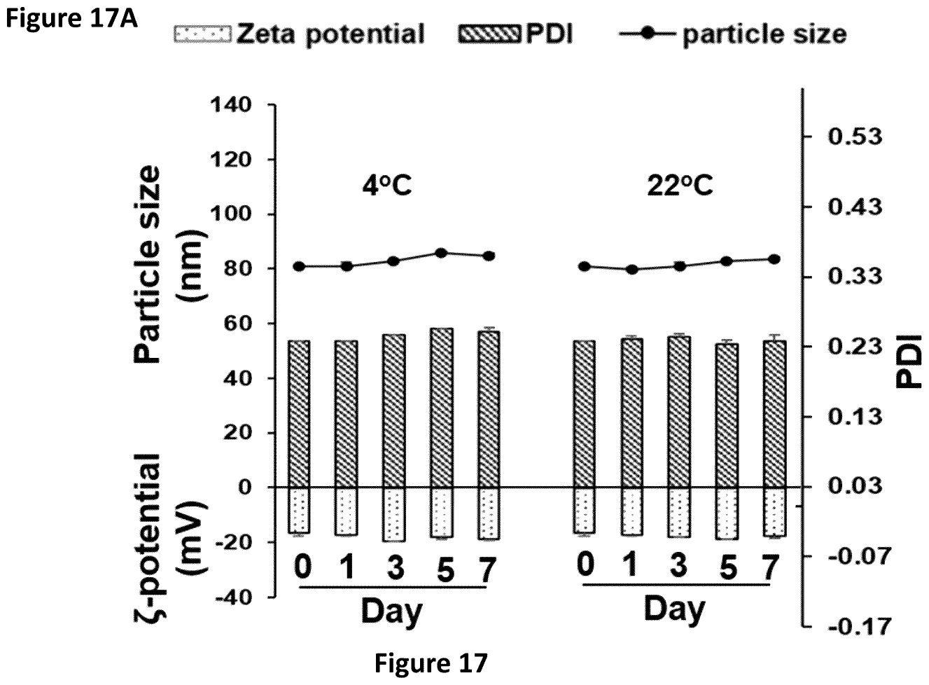

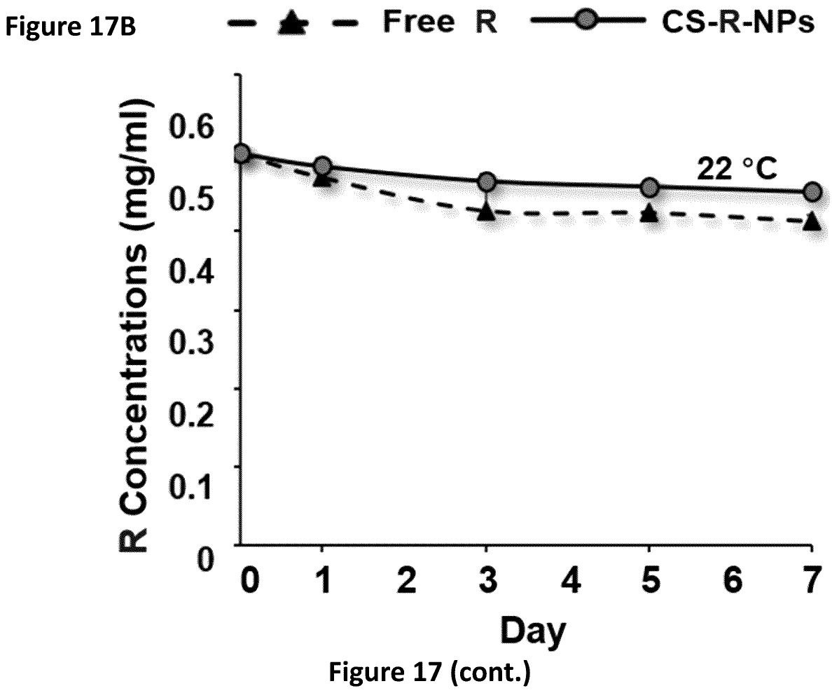

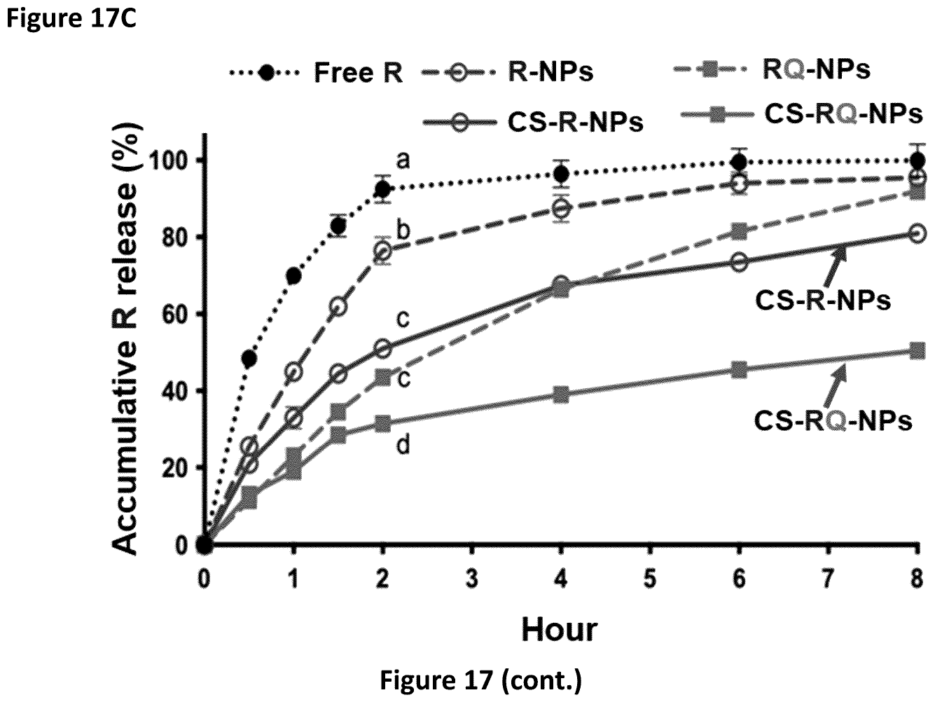

Using R-NPs as an example, the NPs were stable at both 4° C. and 22° C. (FIG. 17A). Nanoencapsulation increased R's stability (FIG. 17B). Similarly, Q and RQ were also stable at 4° C. and 22° C. after encapsulation. A dialysis method was used to measure R and Q release profiles from NPs at 37° C. (FIG. 17C): free R and Q showed burst release while nanoencapsulated R and Q exhibited sustained release. The release profiles of NPs were even slower at 4° C. and 22° C.

### Nanoencapsulation Increased the Antimicrobial Activity of R and Q

### Antibacterial Activity

The antibacterial activity of all NPs and their controls against Salmonella enterica subsp. enterica (S. enterica ATCC 13076), Listeria monocytogenes (L. monocytogenes ATCC 15313), E coli (ATCC 43895) and Staphylococcus aureus subsp. aureus (S. aureus ATCC 25923) were investigated. Free R, free Q, free RQ, R-NPs, or Q-NPs, or RQ-NPs with or without 1% CS were added to bacterial suspension, incubated at 37° C. with constant shaking at 100 rpm overnight, and the optical density (absorbance) at 595 nm was measured using a plate reader. Free RQ, RQ-NPs, and CS-RQ-NPs contain 240 μg/mL RQ (150 μg/mL R+90 μg/mL Q); all the rest treatments contained 300 μg/mL R or Q.

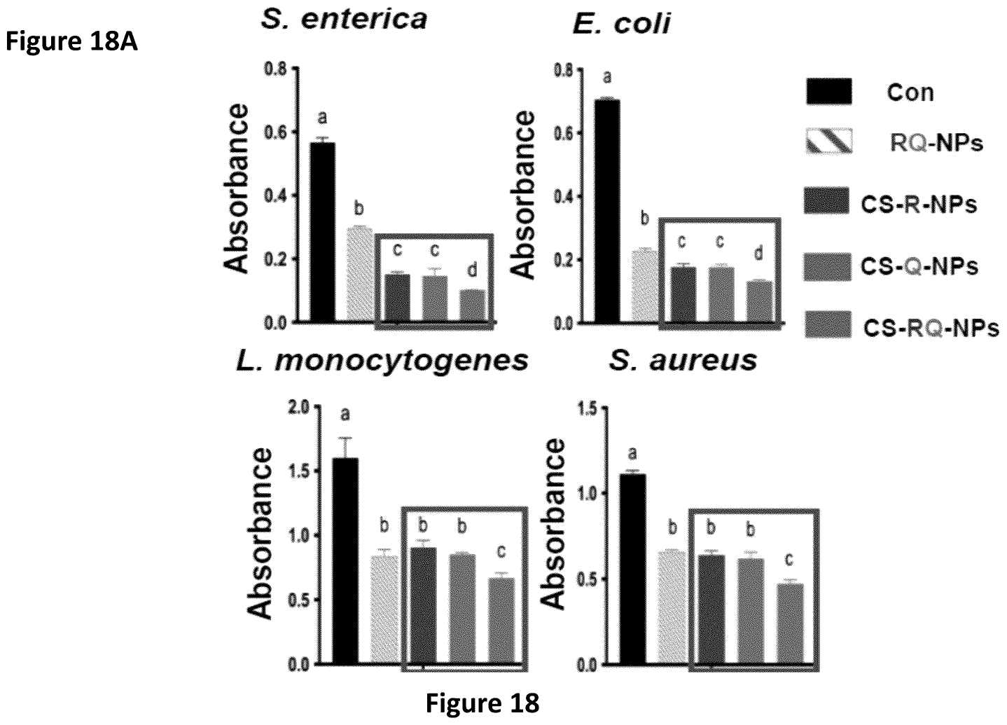

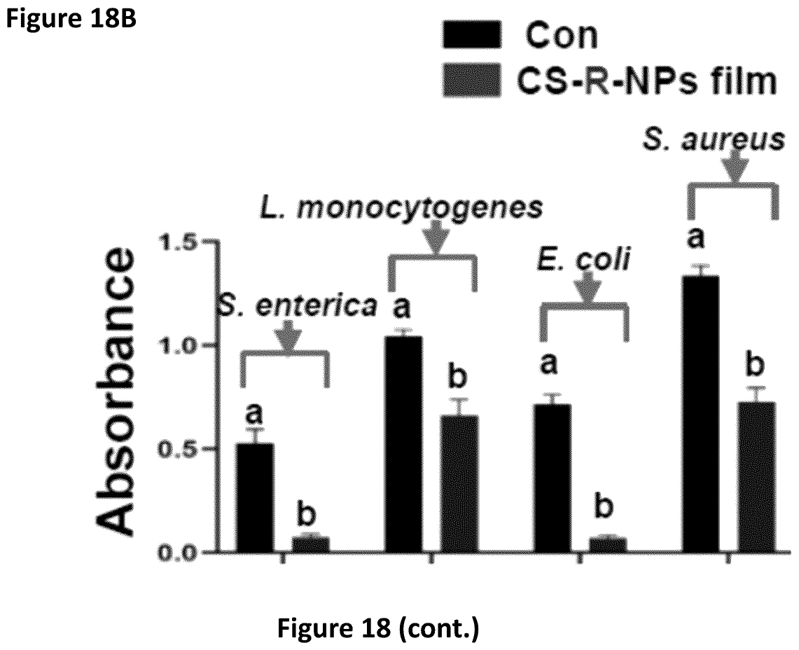

Nanoencapsulation increased R and Q's antibacterial activity. RQ-NPs showed a strong antibacterial activity (FIG. 18). When CS was combined with R-NPs, Q-NPs, the antibacterial activity was significantly increased against S. enterica (FIG. 18). Importantly, CS-RQ-NPs compared to CS-R-NPs or CS-Q-NPs had significantly higher inhibitory effects on S. enterica, E. coli, L. monocytogenes, and S. aureus (FIG. 18), even though RQ concentration (240 μg/mL) was lower than R alone or Q alone (300 μg/mL). The data indicated that R and Q have a synergistical inhibitory effect on S. enterica, E. coli, L. monocytogenes, and S. aureus, when encapsulated into NPs, and coating the RQ-NPs with CS.

CS-R-NPs was dried in a biosafety cabinet to make its sterile films of 0.008 mm thickness that were added into S. enterica, E. coli, L. monocytogenes, and S. aureus solutions. After incubation with shaking at 37° C. at 100 rpm overnight, the dried CS-R-NPs film still had a significant antibacterial activity against the four bacteria.

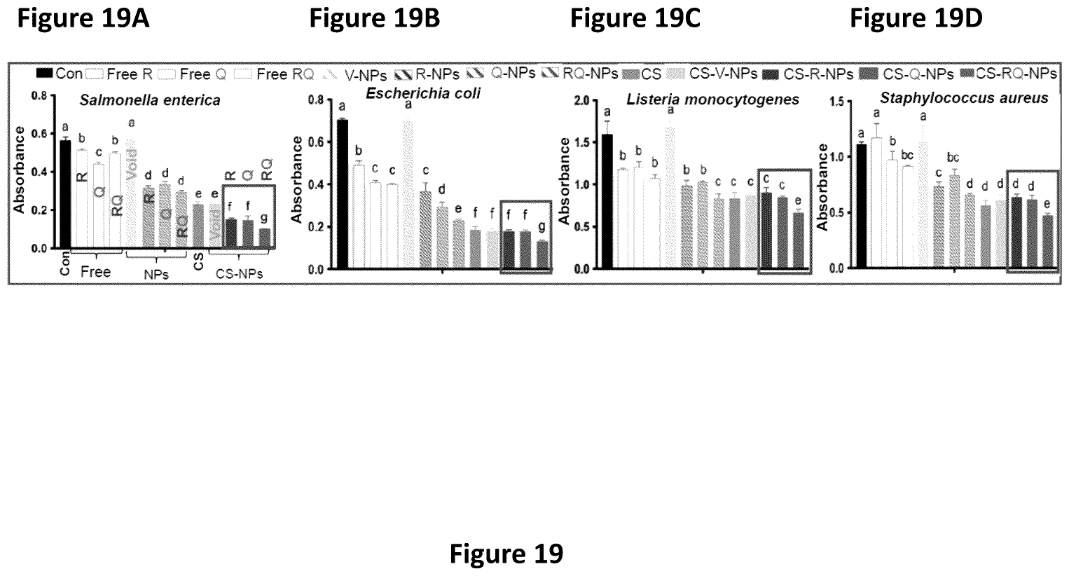

Void-NPs (V-NPs) had no antibacterial activity against the four bacteria (FIG. 19A through FIG. 19D). Free R, free Q, and free RQ inhibited growth of E. coli and L. monocytogenes (FIG. 19B and FIG. 19C), but the inhibitory effects were weaker than R-NPs, Q-NPs, and RQ-NPs.

### Antibacterial Mechanisms

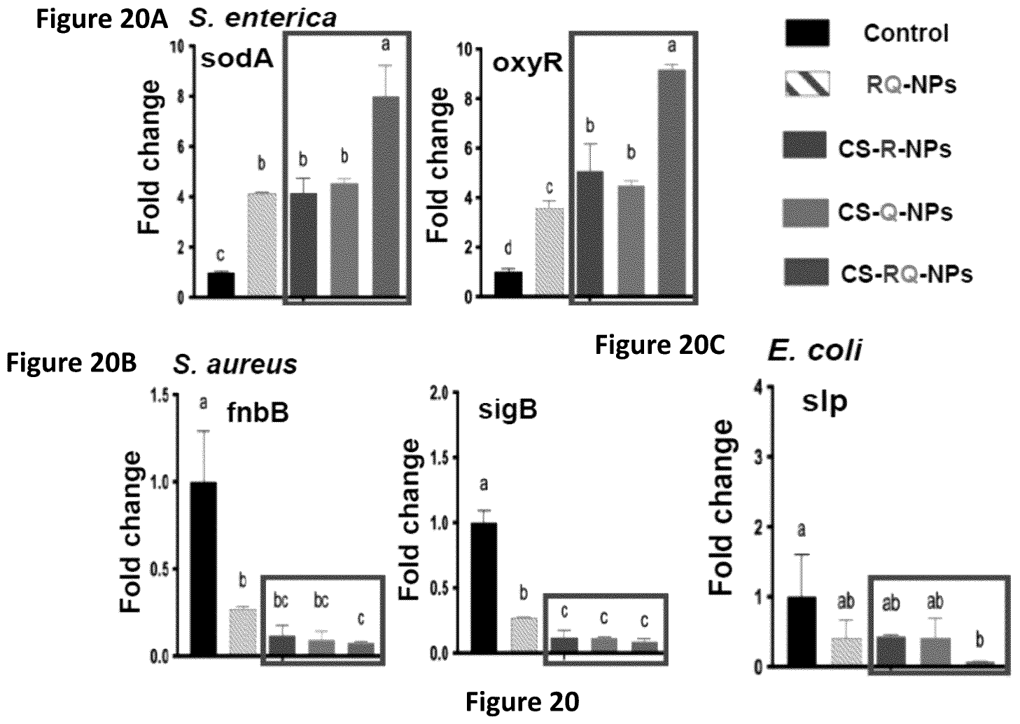

With a nanoscale size, NPs interact with bacterial membranes, reduce their resistance to antibiocides and cause cell death, thus demonstrated efficacy and effectiveness against microbes. To understand the biocidal mechanisms of the NPs, studies were conducted on bacterial gene expression analysis after treatment. Bacterial RNA was extracted, and gene expression related to oxidative stress, virulence, bacterial membrane function, and biofilm formation were measured using real-time PCR. CS-RQ-NPs significantly increased the expression of soda (encoding manganese superoxide dismutase) and oxyR (regulating hydrogen peroxide detoxification) in S. enterica (FIG. 20A), which indicated CS-RQ-NPs treatment increased oxidative stress, as stress-response genes were upregulated. CS-RQ-NPs also significantly decreased expression of fnbB (membrane function-related gene) and sigB (biofilm-related gene) in S. aureus (FIG. 20B), and slp (outer membrane protein) expression in E. coli (FIG. 20C), which indicated CS-RQNPs treatment decreased bacterial membrane function, and inhibited biofilm formation.

### NP-Based Coating Increased Strawberry Shelf-Life

Strawberries were randomly divided into the following seven treatment groups: 1) control, 2) R-NPs, 3)CS-R-NPs, 4) Q-NPs, 5)CS-Q-NPs, 6) RQ-NPs, and 7)CS-RQ-NPs. Each group was dipped into its solution for 1 minute and placed upright into a plastic container at RT (22° C.). The changes of the seven groups were visually obvious. The control group (no treatment) had decayed on day 3. The R-NPs and Q-NPs treated groups showed signs of quality deterioration and rotted on day 5. In contrast, the RQ-NPs, and particularly CS-R-NPs, CS-Q-NPs, and CS-RQ-NPs protected strawberries very well by maintaining their freshness. The prolonged shelf life was partially due to reduced yeast and mold growth on NP-treated strawberries. Humidity was consistent.

### The Coating Preserves the Strawberry Nutrition Values and Maintained the Freshness

The nutrition values of strawberries at day 0 (before treatment) and at day 5 after coating with CS or CS-R-NPs and being stored at RT were also measured. Vitamin C was extracted by 10% meta-phosphoric acid and detected using an UHPLC system. Strawberries treated with CS—R-NPs had 3-fold higher vitamin C content than the control (Table 6).

TABLE 6
Representative results demonstrating the levels of vitamin C, total phenolic content, total
antioxidant capacity, weight loss, and firmness of strawberries coated with various NPs.

| | | | Total | Total | | |
|---|---|---|---|---|---|---|
| | | | phenolic | antioxidant | | |
| | | Vitamin C | content (mg | capacity | Weight | |
| | | (mg/100 g | GAE/100 g | (Trolox | loss | Firmness |
| | Treatment | strawberries) | strawberries) | equivalents/μL) | (%) | (N/mm²) |
| Day | Before | 70.6 ± 6.6^{a} | 182.4 ± 22.2^{a} | 0.46 ± 0.04^{a} | 0 | 0.113 ± 0.018^{a} |
| 0 | treatment | | | | | |
| Day | Control | 15.6 ± 0.8^{c} | 44.1 ± 12.3^{d} | 0.12 ± 0.01^{c} | 64.4 ± 2.4^{a} | 0.021 ± 0.001^{b} |
| 5 | CS | 40.7 ± 13.0^{b} | 105.9 ± 14.8^{c} | 0.25 ± 0.07^{b} | 45.1 ± 0.7^{b} | 0.093 ± 0.004^{a} |
| | CS-R-NPs | 48.0 ± 7.8^{b} | 158.0 ± 5.4^{b} | 0.30 ± 0.04^{b} | 41.7 ± 1.3^{b} | 0.107 ± 0.002^{a} |

Three independent experiments were conducted.
Values are means ± SD.
Column values without sharing a common letter significantly different at p < 0.05.

Total polyphenol content was extracted using a Folin-Ciocalteu method and detected using a 96-well microplate reader at 765 nm. Gallic acid was used as a standard. Treated strawberries only lost 13% of total polyphenol content after 5 days as compared to 76% loss from the untreated strawberries (Table 6). Total antioxidant capacity was determined using a total antioxidant capacity assay kit (Sigma MAK187). The CS-R-NPs-treated and untreated control strawberries had 65% and 26% of the initial total antioxidant capacity, respectively, after storing for 5 days (Table 6). CS-R-NP-treated strawberries had the least weight loss and highest firmness among all groups (Table 6).

### Encapsulation Improved Q Oral Bioavailability

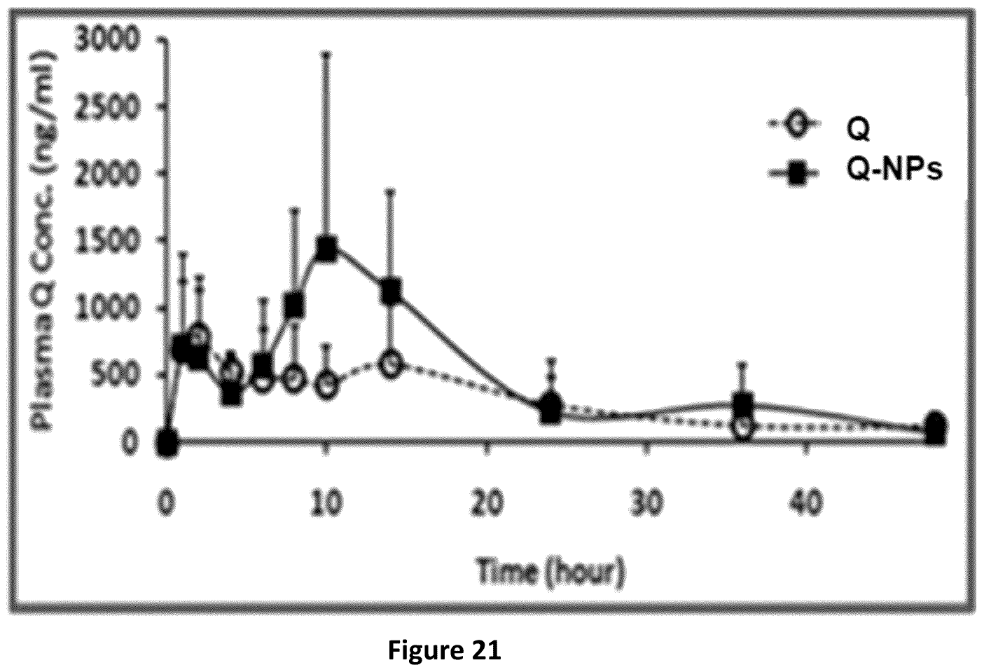

Male Sprague-Dawley rats (n=3 per group) weighing 200-250 g and the rounded tips catheters were surgically implanted into the jugular vein of the rats with the benefits of multiple blood draw. Free Q and Q-NPs were given to male SD rats at a dose 50 mg/kg body weight. Blood was collected via the catheters at hour 0, 1, 2, 3, 4, 6, 8, 10, 14, 24, 36, and 48 into heparinized tubes. Blood Q concentrations were measured using an HPLC system (FIG. 21). Pharmacokinetic parameters were determined on each individual set of data by using a non-compartmental model. Maximum plasma concentration (Cₘₐₓ) and time of maximum plasma concentration (Tₘₐₓ) were obtained from direct blood Q concentration measurement, and the area under the concentration-time curve from time zero to time 24 h (AUC_{0-24 h}) was calculated using the trapezoidal method. The area under the plasma concentration time curve from time zero to infinity (AUC_{0→∞}) was calculated as AUC_{0→24 h}+Cₜ/Kₑ, where Cₜ was the Q concentration observed at the previous time, and Ke is the apparent elimination rate constant obtained from the terminal slope of the individual plasma concentration-time curves after logarithmic transformation of the plasma concentration values and application of linear regression. The relative bioavailability Fr at infinity was calculated as Fr=AUC_{Q-NP, 0→∞}/AUC_{Q, 0→∞}. The half-life (t_{1/2})=0.693/k. The mean residence time (MRT)=1/k. Q-NPs had about a 2-fold increase in blood Q concentrations and their relative bioavailability compared to free Q (Table 7).

TABLE 7
Representative results demonstrating the pharmacokinetic
parameters and bioavailability of Q after oral administration
of free Q and Q-NLCs to rats.

| Parameter | Q | Q-NLC |
|---|---|---|
| Cₘₐₓ (ng · ml⁻¹) | 963.9 ± 535.9 | 1897.4 ± 1339.4* |
| Tₘₐₓ (h) | 2.00 ± 0.00 | 10.00 ± 0.00 |
| Kₑ (h⁻¹) | 0.07 ± 0.06 | 0.04 ± 0.02 |
| T_{1/2} (h) | 16.7 ± 10.5 | 22.7 ± 10.2* |
| MRT (h) | 12.1 ± 15.1 | 32.7 ± 14.8* |
| AUC_{o→24 h} (ng · h · ml⁻¹) | 14632.0 ± 6267.4 | 26314.5 ± 8462.3* |
| AUC_{o→∞} (ng · h · ml⁻¹) | 15273.0 ± 6090.6 | 30285.6 ± 10140.9* |
| Fᵣ | — | 1.98 |

In summary, the present studies have developed a phytochemical nanoencapsulation-based multifunctional edible coating on fresh fruits and vegetables with triplet functions. As a protective layer, the coating acts as antimicrobial and antioxidant agents to maintain the quality and nutrition values of fresh fruits and vegetables, extend their shelf life, and reduce the generation of food waste. As an edible layer, the coating on fresh fruits and vegetables acts as a carrier of phytochemical-based nutraceutical supplements to promote health. The nanoencapsulation ensured effective delivery and enhanced body absorption of these phytochemicals before they were degraded during storage and in the gastrointestinal tract. As nutraceutical foods, nanoencapsulated phytochemical-coated fresh fruits and vegetables balance their cost and value, overcome the price barrier of acceptance, and become a popular health diet. The novelty of this project lies, in part, in the application of edible coatings on fresh fruits and vegetables as delivery vehicles of nanoencapsulated phytochemicals, while these phytochemicals provided antimicrobial/antioxidant protection functions to fresh fruits and vegetables. With increased nutrient value, extended shelf life, and improved food safety, functionalized fresh fruits and vegetables can become a popular health diet.

### Example 4: Exemplary Embodiments

### Embodiment 1

A nanoparticle comprising a) at least one phytochemical or bioactive compound, wherein the phytochemical or bioactive compound is present in a concentration range of about 0.1 wt % to about 50 wt %; b) at least one lipid, wherein the lipid is present in a concentration range of about 5 wt % to about 90 wt %; c) at least one surfactant, wherein the surfactant is present in a concentration range of about 10 wt % to about 75 wt %; and d) at least one vitamin E, wherein the vitamin E is present in a concentration range of about 5 wt % to about 75 wt %; wherein the nanoparticle is an antimicrobial nanoparticle.

### Embodiment 2

The nanoparticle of embodiment 1, wherein the at least one phytochemical or bioactive compound is selected from the group consisting of resveratrol, quercetin, curcumin, theaflavins, thearubigins, epigallocatechin gallate (EGCG), (+)-catechin, (−)-epicatechin, (−)-epicatechin gallate, (−)-epigallocatechin, (+)-gallocatechin, isorhamnetin, kaempferol, myricetin, apigenin, luteolin, baicalein, chrysin, forskolin, chlorophyll a, chlorophyll b, eriodictyol, hesperetin, naringenin, taxifolin, catechins, luteolin, cyanidin, genistein, daidzein, genistein, glycitein, biochanin A, formononetin, cyanidin, delphinidin, malvidin, pelargonidin, peonidin, petunidin, proanthocyanidins, α-carotene, β-carotene, β-cryptoxanthin, lutein, zeaxanthin, lycopene, and any combination thereof.

### Embodiment 3

The nanoparticle of embodiment 1, wherein the at least one phytochemical or bioactive compound is present in a concentration of about 10 wt %.

### Embodiment 4

The nanoparticle of embodiment 4, wherein the at least one lipid is selected from the group consisting of a fatty acid, wax, sterol, lipid-soluble vitamin, vitamin A, vitamin D, vitamin E, vitamin K, monoglyceride, diglyceride, triglyceride, phospholipid, L-α-phosphatidylcholine (PC), soy PC, egg PC, phosphatidic acid (PA), phosphatidylethanolamine (PE), lecithin, phosphatidylserine (PS), phosphoinositide, phosphatidylinositol (PI), phosphatidylinositol phosphate (PIP), phosphatidylinositol bisphosphate (PIP2), phosphatidylinositol trisphosphate (PIP3), phosphosphingolipid, sphingolipid, ceramide phosphorylcholine (SPH), ceramide phosphorylethanolamine (Cer-PE), ceramide phosphoryl lipid, and any combination thereof.

### Embodiment 5

The nanoparticle of embodiment 5, wherein the at least one lipid is present in a concentration of about 58 wt %.

### Embodiment 6

The nanoparticle of embodiment 1, wherein the at least one surfactant is selected from the group consisting of a polyethylene glycol (PEG), functionalized PEG, polyethylene glycol 15-hydroxystearate, Tween 80, phospholipids, PEG40-stearate, PEG100-stearate, PEG (10-1000)-fatty acid, mustard, lecithin, soy lecithin, egg lecithin, monoglycerides, diglycerides, polysorbates, carrageenan, guar gum, canola oil, polysorbates (Tween™), sodium dodecyl sulfate, lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Byrj 52, Byrj S 100, Brij 721™, bile salts, sodium deoxycholate, sodium cholate, polyoxyl castor oil (Cremophor™), nonylphenol ethoxylate (Tergitol™), cyclodextrins, methylbenzethonium chloride (Hyamine™), and any combination thereof.

### Embodiment 7

The nanoparticle of embodiment 6, wherein the at least one surfactant is polyethylene glycol 15-hydroxystearate.

### Embodiment 8

The nanoparticle of embodiment 1, wherein the at least one surfactant is in a concentration of about 40 wt %.

### Embodiment 9

The nanoparticle of embodiment 1, wherein the at least one vitamin E is selected from the group consisting of α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, δ-tocotrienol, δ-tocotrienol acetate, δ-tocotrienol nicotinate, and any combination thereof.

### Embodiment 10

The nanoparticle of embodiment 1, wherein the at least one vitamin E is in a concentration of about 40 wt %.

### Embodiment 11

The nanoparticle of embodiment 1, wherein the nanoparticle further comprises at least one coating agent.

### Embodiment 12

The nanoparticle of embodiment 11, wherein the nanoparticle is coated with the at least one coating agent.

### Embodiment 13

The nanoparticle of embodiment 12, wherein the nanoparticle is coated with chitosan, methyl cellulose, hydroxypropyl methyl cellulose, or any combination thereof.

### Embodiment 14

The nanoparticle of embodiment 11, wherein the at least one coating agent is selected from the group consisting of a chitosan, starch, stabilizer, plasticizer, lipid, polysaccharide, protein, zein, soy protein, whey, casein, fatty acid, wax, neutral lipid, resin, cellulose, methyl cellulose, hydroxypropyl cellulose, hydroxypropyl methyl cellulose, alginate, and any combination thereof.

### Embodiment 15

The nanoparticle of embodiment 1, wherein the nanoparticle further comprises at least one nutritional or bioactive agent.

### Embodiment 16

The nanoparticle of embodiment 1, wherein the nanoparticle reduces or inhibits the activity or level of at least one microorganism.

### Embodiment 17

The nanoparticle of embodiment 16, wherein the microorganism is selected from the group consisting of a bacterium, virus, pathogen, parasite, fungus, yeast, mold, and any combination thereof.

### Embodiment 18

The nanoparticle of embodiment 17, wherein the bacterium is selected from the group consisting of Escherichia, Escherichia coli, Salmonella enterica, Staphylococcus, Staphylococcus aureus, Listeria monocytogenes, Clostridium perfringens, Clostridium botulinum, Shigella boydii, Vibrio, Vibrio parahaemolyticus, Campylobacter, Campylobacter jejuni, Yersinia, Yersinia enterocolitica, Cronobacter sakazakii, Enterobacteriaceae, Erwinia herbicola, Rahnella aquatilis, Lacticaseibacillus casei, Leuconostoc mesenteroides, Bacillus cereus, Pseudomonadaceae, P. fluorescens, and any combination thereof.

### Embodiment 19

The nanoparticle of embodiment 17, wherein the yeast is selected from the group consisting of Candida sp., Candida pulcherrima, Candida humilis, Candida milleri, Candida tropicalis, Candida fermentati, Candida krusei, Candida parapsilosis, Candida guilliermondii, Candida lusitaniae, Candida rugosa, Cryptococcus sp., Rhodotorula sp., Trichosporon sp., Pichia sp., Torulaspora sp., C. lambica, C. sake, Debaryomyces polymorphus, and any combination thereof.

### Embodiment 20

The nanoparticle of embodiment 17, wherein the mold is selected from the group consisting of Botrytis, Botrytis cinerea, Rhizopus, Rhizopus stolonifer, Mucor, Mucor piriformis, Rhizoctonia solani, Phytophtora cactorum, Alternaria, Penicillium, Cladosporium, Aspergillus, Fusarium, Geotrichum, and any combination thereof.

### Embodiment 21

The nanoparticle of embodiment 1, wherein the nanoparticle is a biodegradable edible nanoparticle.

### Embodiment 22

The nanoparticle of embodiment 1, wherein the nanoparticle is selected from the group consisting of a liposome, nanoemulsion, micelle, solid lipid nanoparticle (SLN), nanostructured lipid carrier (NLC), modified lipid nanoparticle, and any combination thereof.

### Embodiment 23

The nanoparticle of embodiment 1, wherein the nanoparticle preserves at least one nutrient.

### Embodiment 24

The nanoparticle of embodiment 23, wherein the at least one nutrient is selected from the group consisting of vitamin, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, and any combination thereof.

### Embodiment 25

A composition comprising at least one nanoparticle of any one of embodiments 1-24.

### Embodiment 26

The composition of embodiment 25, wherein the composition is selected from the group consisting of an edible coating, packing material, food preparation element, and any combination thereof.

### Embodiment 27

The composition of embodiment 25, wherein the composition preserves at least one nutrient.

### Embodiment 28

The composition of embodiment 27, wherein the at least one nutrient is selected from the group consisting of vitamin, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, and any combination thereof.

### Embodiment 29

A method of reducing or inhibiting the activity or level of at least one microorganism on a surface of an element, wherein the method comprises administering an effective amount of at least one nanoparticle of any one of embodiments 1-24 or a composition thereof to the surface of the element.

### Embodiment 30

A method of preventing or reducing the growth of at least one microorganism on a surface of an element, wherein the method comprises administering an effective amount of at least one nanoparticle of any one of embodiments 1-24 or a composition thereof to the surface of the element.

### Embodiment 31

The method of embodiment 29 or 30, wherein the element is selected from the group consisting of food, packing material, food preparation element, and any combination thereof.

### Embodiment 32

The method of embodiment 29 or 30, wherein the at least one microorganism is selected from the group consisting of a bacterium, virus, pathogen, parasite, fungus, yeast, mold, and any combination thereof.

### Embodiment 33

The method of embodiment 32, wherein the bacterium is selected from the group consisting of Escherichia, Escherichia coli, Salmonella enterica, Staphylococcus, Staphylococcus aureus, Listeria monocytogenes, Clostridium perfringens, Clostridium botulinum, Shigella boydii, Vibrio, Vibrio parahaemolyticus, Campylobacter, Campylobacter jejuni, Yersinia, Yersinia enterocolitica, Cronobacter sakazakii, Enterobacteriaceae, Erwinia herbicola, Rahnella aquatilis, Lacticaseibacillus casei, Leuconostoc mesenteroides, Bacillus cereus, Pseudomonadaceae, P. fluorescens, and any combination thereof.

### Embodiment 34

The method of embodiment 32, wherein the yeast is selected from the group consisting of Candida sp., Candida pulcherrima, Candida humilis, Candida milleri, Candida tropicalis, Candida fermentati, Candida krusei, Candida parapsilosis, Candida guilliermondii, Candida lusitaniae, Candida rugosa, Cryptococcus sp., Rhodotorula sp., Trichosporon sp., Pichia sp., Torulaspora sp., C. lambica, C. sake, Debaryomyces polymorphus, and any combination thereof.

### Embodiment 35

The method of embodiment 32, wherein the mold is selected from the group consisting of Botrytis, Botrytis cinerea, Rhizopus, Rhizopus stolonifer, Mucor, Mucor piriformis, Rhizoctonia solani, Phytophtora cactorum, Alternaria, Penicillium, Cladosporium, Aspergillus, Fusarium, Geotrichum, and any combination thereof.

### Embodiment 36

A method of increasing a shelf-life of food, wherein the method comprises administering an effective amount of at least one nanoparticle of any one of embodiments 1-24 or a composition thereof to a surface of at least one selected from the group consisting of food, packing material, food preparation element, and any combination thereof.

### Embodiment 37

A method of preserving nutrients in food, wherein the method comprises administering an effective amount of at least one nanoparticle of any one of embodiments 1-24 or a composition thereof to a surface of at least one selected from the group consisting of food, packing material, food preparation element, and any combination thereof.

### Embodiment 38

The method of embodiment 37, wherein the at least one nutrient is selected from the group consisting of vitamin, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, and any combination thereof.

### Embodiment 39

A method of coating a surface of an element, wherein the method comprises administering an effective amount of at least one nanoparticle of any one of embodiments 1-24 or a composition thereof to the surface of the element.

### Embodiment 40

The method of embodiment 39, wherein the element is at least one selected from the group consisting of food, packing material, food preparation element, and any combination thereof.

### Embodiment 41

A method of improving nutritional value in food, wherein the method comprises administering an effective amount of at least one nanoparticle of any one of embodiments 1-24 or a composition thereof to a surface of at least one selected from the group consisting of food, packing material, food preparation element, and any thereof.

### Embodiment 42

A method of delivering at least one nutritional or bioactive agent to a subject, wherein the method comprises administering an effective amount of at least one nanoparticle of embodiment 15 or a composition thereof to the subject.

### Embodiment 43

A method of treating or preventing nutrient deficiency in a subject in need thereof, wherein the method comprises administering an effective amount of at least one nanoparticle of any one of embodiments 1-24 or a composition thereof to the subject.

### Embodiment 44

A method of treating or preventing a disease, disorder, or condition in a subject in need thereof, wherein the method comprises administering an effective amount of at least one nanoparticle of any one of embodiments 1-24 or a composition thereof to the subject.

### Embodiment 45

The method of embodiment 46, wherein the disease, disorder, or condition is selected from the group consisting of obesity, diabetes, cancer, neurodegenerative disease, neurodegenerative disorder, disease associated with nutrient deficiency, disorder associated with nutrient deficiency, condition associated with nutrient deficiency, and any combination thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A nanoparticle comprising
a) at least one phytochemical or bioactive compound, wherein the phytochemical or bioactive compound is present in a concentration range of about 0.1 wt % to about 50 wt %;
b) at least one lipid, wherein the lipid is present in a concentration range of about 5 wt % to about 90 wt %;
c) at least one surfactant, wherein the surfactant is present in a concentration range of about 10 wt % to about 75 wt %; and
d) at least one vitamin E, wherein the vitamin E is present in a concentration range of about 5 wt % to about 75 wt %;
wherein the nanoparticle is an antimicrobial nanoparticle.

2. The nanoparticle of claim 1, wherein
a) the at least one phytochemical or bioactive compound is selected from the group consisting of resveratrol, quercetin, curcumin, theaflavins, thearubigins, epigallocatechin gallate (EGCG), (+)-catechin, (−)-epicatechin, (−)-epicatechin gallate, (−)-epigallocatechin, (+)-gallocatechin, isorhamnetin, kaempferol, myricetin, apigenin, luteolin, baicalein, chrysin, forskolin, chlorophyll a, chlorophyll b, eriodictyol, hesperetin, naringenin, taxifolin, catechins, luteolin, cyanidin, genistein, daidzein, genistein, glycitein, biochanin A, formononetin, cyanidin, delphinidin, malvidin, pelargonidin, peonidin, petunidin, proanthocyanidins, α-carotene, β-carotene, β-cryptoxanthin, lutein, zeaxanthin, lycopene, and any combination thereof;
b) the at least one lipid is selected from the group consisting of a fatty acid, wax, sterol, lipid-soluble vitamin, vitamin A, vitamin D, vitamin E, vitamin K, monoglyceride, diglyceride, triglyceride, phospholipid, L-α-phosphatidylcholine (PC), soy PC, egg PC, phosphatidic acid (PA), phosphatidylethanolamine (PE), lecithin, phosphatidylserine (PS), phosphoinositide, phosphatidylinositol (PI), phosphatidylinositol phosphate (PIP), phosphatidylinositol bisphosphate (PIP2), phosphatidylinositol trisphosphate (PIP3), phosphosphingolipid, sphingolipid, ceramide phosphorylcholine (SPH), ceramide phosphorylethanolamine (Cer-PE), ceramide phosphoryl lipid, and any combination thereof;
c) the at least one surfactant is selected from the group consisting of a polyethylene glycol (PEG), functionalized PEG, polyethylene glycol 15-hydroxystearate, Tween 80, phospholipids, PEG40-stearate, PEG100-stearate, PEG (10-1000)-fatty acid, mustard, lecithin, soy lecithin, egg lecithin, monoglycerides, diglycerides, polysorbates, carrageenan, guar gum, canola oil, polysorbates (Tween™), sodium dodecyl sulfate, lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Byrj 52, Byrj S 100, Brij 721™, bile salts, sodium deoxycholate, sodium cholate, polyoxyl castor oil (Cremophor™), nonylphenol ethoxylate (Tergitol™), cyclodextrins, methylbenzethonium chloride (Hyamine™), and any combination thereof;
d) the at least one vitamin E is selected from the group consisting of α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, δ-tocotrienol, δ-tocotrienol acetate, δ-tocotrienol nicotinate, and any combination thereof; or
e) or any combination thereof.

3. The nanoparticle of claim 1, wherein the nanoparticle further comprises at least one coating agent.

4. The nanoparticle of claim 3, wherein the at least one coating agent is selected from the group consisting of a chitosan, starch, stabilizer, plasticizer, lipid, polysaccharide, protein, zein, soy protein, whey, casein, fatty acid, wax, neutral lipid, resin, cellulose, methyl cellulose, hydroxypropyl cellulose, hydroxypropyl methyl cellulose, alginate, and any combination thereof.

5. The nanoparticle of claim 1, wherein the nanoparticle further comprises at least one nutritional or bioactive agent.

6. The nanoparticle of claim 1, wherein the nanoparticle reduces or inhibits the activity or level of at least one microorganism.

7. The nanoparticle of claim 6, wherein the microorganism is selected from the group consisting of a bacterium, virus, pathogen, parasite, fungus, yeast, mold, and any combination thereof.

8. The nanoparticle of claim 6, wherein the microorganism is selected from the group consisting of Escherichia, Escherichia coli, Salmonella enterica, Staphylococcus, Staphylococcus aureus, Listeria monocytogenes, Clostridium perfringens, Clostridium botulinum, Shigella boydii, Vibrio, Vibrio parahaemolyticus, Campylobacter, Campylobacter jejuni, Yersinia, Yersinia enterocolitica, Cronobacter sakazakii, Enterobacteriaceae, Erwinia herbicola, Rahnella aquatilis, Lacticaseibacillus casei, Leuconostoc mesenteroides, Bacillus cereus, Pseudomonadaceae, P. fluorescens, Candida sp., Candida pulcherrima, Candida humilis, Candida milleri, Candida tropicalis, Candida fermentati, Candida krusei, Candida parapsilosis, Candida guilliermondii, Candida lusitaniae, Candida rugosa, Cryptococcus sp., Rhodotorula sp., Trichosporon sp., Pichia sp., Torulaspora sp., C. lambica, C. sake, Debaryomyces polymorphus, Botrytis, Botrytis cinerea, Rhizopus, Rhizopus stolonifer, Mucor, Mucor piriformis, Rhizoctonia solani, Phytophtora cactorum, Alternaria, Penicillium, Cladosporium, Aspergillus, Fusarium, Geotrichum, and any combination thereof.

9. The nanoparticle of claim 1, wherein the nanoparticle is selected from the group consisting of a biodegradable edible nanoparticle, liposome, nanoemulsion, micelle, solid lipid nanoparticle (SLN), nanostructured lipid carrier (NLC), modified lipid nanoparticle, and any combination thereof.

10. The nanoparticle of claim 1, wherein the nanoparticle preserves at least one nutrient.

11. The nanoparticle of claim 10, wherein the at least one nutrient is selected from the group consisting of vitamin, vitamin C, vitamin A, folate, vitamin D, vitamin E, vitamin K, vitamin B complex, vitamin B1, vitamin B2, vitamin B3, vitamin B5, vitamin B6, vitamin B7, vitamin B9, vitamin B12, mineral, antioxidant, and any combination thereof.

12. A composition comprising at least one nanoparticle of claim 1.

13. The composition of claim 12, wherein the composition is selected from the group consisting of an edible coating, packing material, food preparation element, and any combination thereof.

14. A method of preventing or reducing or inhibiting the growth, activity, or level of at least one microorganism on a surface of an element, wherein the method comprises administering an effective amount of at least one nanoparticle of claim 1 or a composition thereof to the surface of the element.

15. A method of increasing a shelf-life of food, wherein the method comprises administering an effective amount of at least one nanoparticle of claim 1 or a composition thereof to a surface of at least one selected from the group consisting of food, packing material, food preparation element, and any combination thereof.

16. A method of preserving nutrients in food, wherein the method comprises administering an effective amount of at least one nanoparticle of claim 1 or a composition thereof to a surface of at least one selected from the group consisting of food, packing material, food preparation element, and any combination thereof.

17. A method of coating a surface of an element,
wherein the method comprises administering an effective amount of at least one nanoparticle of claim 1 or a composition thereof to the surface of the element.

18. A method of improving nutritional value in food, wherein the method comprises administering an effective amount of at least one nanoparticle of claim 1 or a composition thereof to a surface of at least one selected from the group consisting of food, packing material, food preparation element, and any thereof.

19. A method of treating or preventing a disease, disorder, or condition in a subject in need thereof, wherein the method comprises administering an effective amount of at least one nanoparticle of claim 1 or a composition thereof to the subject.

20. A method of delivering at least one nutritional or bioactive agent to a subject, wherein the method comprises administering an effective amount of at least one nanoparticle of claim 5 or a composition thereof to the subject.
